SEQUENCE LISTING (25 sequences)

SEQ ID NO: 1 - DNA, 21 bp, synthetic construct
  misc_feature 1..21: note = CytoB_B3
  source 1..21: mol_type = other DNA
gtttgcatgtagatagcgaat

SEQ ID NO: 2 - DNA, 49 bp, synthetic construct
  misc_feature 1..49: note = CytoB_BIP
  source 1..49: mol_type = other DNA
agcaatacattacacatcagacacattttatccgtaatttacgtctcga

SEQ ID NO: 3 - DNA, 19 bp, synthetic construct
  misc_feature 1..19: note = F3_Set 1
  source 1..19: mol_type = other DNA
ttgtgatcaactccgcgaa

SEQ ID NO: 4 - DNA, 25 bp, synthetic construct
  misc_feature 1..25: note = B3_Set 1
  source 1..25: mol_type = other DNA
tcaattaaattgtcatcttcgtcct

SEQ ID NO: 5 - DNA, 19 bp, synthetic construct
  misc_feature 1..19: note = LF_Set 1
  source 1..19: mol_type = other DNA
ctgcacttacaccgcaaac

SEQ ID NO: 6 - DNA, 23 bp, synthetic construct
  misc_feature 1..23: note = LB_Set 1
  source 1..23: mol_type = other DNA
atgataaagtagctggttttgct

SEQ ID NO: 7 - DNA, 41 bp, synthetic construct
  misc_feature 1..41: note = FIP_Set 1
  source 1..41: mol_type = other DNA
tcagtactagtgcctgtgccgtcagctgatgcacaatcgtt

SEQ ID NO: 8 - DNA, 46 bp, synthetic construct
  misc_feature 1..46: note = BIP_Set 1
  source 1..46: mol_type = other DNA
tgtcgtatacagggcttttgacatcttggaagcgacaacaattagt

SEQ ID NO: 9 - DNA, 20 bp, synthetic construct
  misc_feature 1..20: note = F3_Set 2
  source 1..20: mol_type = other DNA
accaatagcagtccagatga

SEQ ID NO: 10 - DNA, 21 bp, synthetic construct
  misc_feature 1..21: note = B3_Set 2
  source 1..21: mol_type = other DNA
cacgattgcagcattgttagc

SEQ ID NO: 11 - DNA, 25 bp, synthetic construct
  misc_feature 1..25: note = LF_Set 2
  source 1..25: mol_type = other DNA
ggactgagatctttcattttaccgt

SEQ ID NO: 12 - DNA, 21 bp, synthetic construct
  misc_feature 1..21: note = LB_Set 2
  source 1..21: mol_type = other DNA
actgagggagccttgaataca

SEQ ID NO: 13 - DNA, 41 bp, synthetic construct
  misc_feature 1..41: note = FIP_Set 2
  source 1..41: mol_type = other DNA
tctggcccagttcctaggtagtccagacaaattcgtggtgg

SEQ ID NO: 14 - DNA, 42 bp, synthetic construct
  misc_feature 1..42: note = BIP_Set 2
  source 1..42: mol_type = other DNA
ggacttccctatggtgctaacaaacgggtgccaatgtgatct

SEQ ID NO: 15 - DNA, 18 bp, synthetic construct
  misc_feature 1..18: note = GAPDH_ID:3_F3
  source 1..18: mol_type = other DNA
gatgctggcgctgagtac

SEQ ID NO: 16 - DNA, 19 bp, synthetic construct
  misc_feature 1..19: note = GAPDH_ID:3_B3
  source 1..19: mol_type = other DNA
gctaagcagttggtggtgc

SEQ ID NO: 17 - DNA, 40 bp, synthetic construct
  misc_feature 1..40: note = GAPDH_ID:3_FIP
  source 1..40: mol_type = other DNA
cttttggctcccccctgcaaatggagtccactggcgtctt

SEQ ID NO: 18 - DNA, 41 bp, synthetic construct
  misc_feature 1..41: note = GAPDH_ID:3_BIP
  source 1..41: mol_type = other DNA
tctgctgatgcccccatgttcggaggcattgctgatgatct

SEQ ID NO: 19 - DNA, 19 bp, synthetic construct
  misc_feature 1..19: note = GAPDH_ID:3_LF
  source 1..19: mol_type = other DNA
agccttctccatggtggtg

SEQ ID NO: 20 - DNA, 21 bp, synthetic construct
  misc_feature 1..21: note = GAPDH_ID:3_LB
  source 1..21: mol_type = other DNA
gtcatgggtgtgaaccatgag

SEQ ID NO: 21 - DNA, 18 bp, synthetic construct
  misc_feature 1..18: note = CytoB_F3
  source 1..18: mol_type = other DNA
attcattgacctcccagc

SEQ ID NO: 22 - DNA, 24 bp, synthetic construct
  misc_feature 1..24: note = CytoB_LF
  source 1..24: mol_type = other DNA
caagattaggcagatgcctaagag

SEQ ID NO: 23 - DNA, 25 bp, synthetic construct
  misc_feature 1..25: note = CytoB_LB
  source 1..25: mol_type = other DNA
acaacagctttctcatcagttacac

SEQ ID NO: 24 - DNA, 48 bp, synthetic construct
  misc_feature 1..48: note = CytoB_FIP
  source 1..48: mol_type = other DNA
aagaacaggcctgttaggatttgttttatcatgatgaaacttcggttc

SEQ ID NO: 25 - DNA, 1793 bp, synthetic construct
  misc_feature 1..1793: note = internal control template
  source 1..1793: mol_type = other DNA
tcaacaacgcattcattgacctcccagccccctcaaacatctcatcatgatgaaacttcggttccctcttaggcatctgcctaatcttgcaaatcctaacaggcctgttcttagcaatacattacacatcagacacaacaacagctttctcatcagttacacacatttgtcgagacgtaaattacggatgagttattcgctatctacatgcaaacggagcatccatattctttatttgcctattcatccacgtaggccgaggtctatactacggatcctatatattcctagaaacatgaaacattggagtagtcctactatttaccgttatagcaacagccttcataggctacgtcctgccctgaggacaaatatcattctgaggagctacggtcatcacaaatctactatcagctatcccttatatcggaacagacctcgtagaatgaatctgagggggcttttccgtcgacaaagcaaccctcacacgattcttcgccttccactttatcctgccattcatcattaccgccctcgcagccgtacatctcctattcctgcacgaaaccggatccaacaaccctaccggaacatgtacatattattattaatattacatagtacatatcattattgatcgtacatagcacatatcatgtcaaataactccagtcaacatgcgtatcaccaccattagatcacgagcttaattaccatgccgcgtgaaaccagcaacccgcttggcagggatccctcttctcgctccgggcccataaatcgtgggggtttctattgatgaactttaacaggcatctggttcttacttcaggaccatctcatctaaaatcgcccactctttccccttaaataagacatctcgatggactaatgactaatcagcccatgctcacacataactgaggtttcatacatttggtattttttaatttttggggatgcttggactcagccatggccgtcaaaggccctaacacagtcaaatcaattgtagctggacttcatggaactcatgatccggcacgacaatccaaacaaggtgctattcagtcaatggttacgggacataacgtgcgtacacgcgcatataagcaggtaaattattagctcattcaaaccccccttaccccccattaaacttatgctctacacaccctataacgccttgccaaaccccccggtgagaatgccctccagatcctaaagatcaaaaggagcaggtatcaagcacacctataacggtagctcataacgccttgctcaaccacacccccacgggaaacagcagtgataaaaattaagccatgaacgaaagtttgactaagttatattaattagagttggtaaatctcgtgccagccaccgcggtcatacgattaacccaaattaatagatccacggcgtaaagagtgtttaagaaaaaaaaaccacaatagagttaaattataactaagctgtaaaaagccctagttaaaataaaataacccacgaaagtgactctaataatcctgacacacgatagctaggacccaaactgggattagataccccactatgcctagccctaaacccaaatagttacataacaaaactattcgccagagtactactcgcaactgcctaaaactcaaaggacttggcggtgcttcacatccacctagaggagcctgttctataatcgataaaccccgatagaccttaccaacccttgccaattcagcctatataccgccatcttcagcaaaccctaaaaaggaacaatagtaagcacaatc

(19) United States
(12) Patent Application Publication
(10) Pub. No.: US 2025/0092439 A1
(43) Pub. Date: Mar. 20, 2025

(54) LYOPHILISED NUCLEIC ACID AMPLIFICATION REACTION COMPOSITION

(71) Applicant: IMPERIAL COLLEGE INNOVATIONS LIMITED, London (GB)

(72) Inventors: Jesus RODRIGUEZ MANZANO, London (GB); Oliver STRINGER, London (GB); Matthew CAVUTO, London (GB); Kenny MALPARTIDA-CARDENAS, London (GB)

(21) Appl. No.: 18/728,028

(22) PCT Filed: Jan. 10, 2023
(86) PCT No.: PCT/GB2023/050033

§ 371 Date: Jul. 10, 2024

(30) Foreign Application Priority Data
Jan. 10, 2022 (GB) 2200234.9

(51) Int. Cl. C12Q 1/6806

(52) U.S. Cl. CPC C12Q 1/6806

(57) ABSTRACT

The present disclosure provides a lyophilized composition for use in a colorimetric nucleic acid amplification reaction. Also provided are processes for producing a lyophilized composition and methods for determining the presence of a target nucleic acid in a test sample or diagnosing or treating infection by a pathogen in a subject.

### CROSS-REFERENCE TO RELATED APPLICATIONS

This application is a National Stage Application of PCT/GB2023/050033, filed Jan. 10, 2023, which claims benefit of priority to Application No. 2200234.9, filed Jan. 10, 2022 in Great Britain, and which applications are incorporated herein by reference. To the extent appropriate, a claim of priority is made to each of the above-disclosed applications.

### SEQUENCE STATEMENT

This application contains a Sequence Listing, which was submitted electronically in TXT format and is hereby incorporated by reference in its entirety. Said TXT copy, created and filed in the pending application has the file name “2024-07-08_149980046FPWO_SEQ_LISTING.xml” and a file size of 34,384 bytes (33.5 KB).

### FIELD OF APPLICATION

The present invention relates to a lyophilized composition for use in a colorimetric nucleic acid amplification reaction.

### BACKGROUND

The provision of point-of care diagnostics for high sensitivity and specificity detection of nucleic acid molecules is of great importance for diagnosis of infectious diseases, such as SARS-CoV-2. Currently, there are a limited number of point-of-care diagnostic devices commercially available, many of which are expensive and rely on highly skilled operators.

Nucleic acid amplification techniques, such as PCR, provide high sensitivity and target specificity, but are time-consuming, require skilled technicians and laboratory resources, so are not ideal in all settings.

Isothermal amplification methods involve the amplification of nucleic acids (DNA or RNA) at a constant temperature by means of enzymes, such as DNA polymerase, and other reagents which are part of a reaction mixture. Isothermal amplification methods, such as loop-mediated isothermal amplification (LAMP) are less demanding of resources than other amplification methods such as PCR. Loop-mediated isothermal amplification (LAMP) has been developed as a rapid, sensitive and simple technique for the detection of pathogens. LAMP methods do not require thermocycling and instead can be run with a simple heat source, such as a heat block. The LAMP technique amplifies nucleic acids in a highly specific, efficient and rapid manner while under isothermal conditions (Notomi et al., Nucleic Acids Research, 28(12), E63, 2000). The technique is based on the principle of auto-cycling and strand displacement DNA synthesis, using a DNA polymerase and a set of specially designed primers that recognise six distinct regions on a target nucleic acid template. LAMP products can be measured in different ways, including measuring turbidity, or use of pH-sensitive dyes or metal indicators. Isothermal amplification methods are described, for example, in WO 2017/079696, WO 2019/234252, WO 2019/234252 and Garcia-Bernalt Diego, J et al, Diagnostics 2021, 11, 438.

Previously, Rodriguez-Manzano et al. (ACS Nano 2016, 10, 3, 3102-3113) reported a methodology for amplification of RNA and DNA in the presence of a colorimetric indicator which would allow for detection of the presence or absence of an amplification reaction via a mobile phone camera at the point-of-care.

The provision of lyophilized reaction mixtures containing all reagents necessary to carry out a colorimetric nucleic acid amplification reaction, is desirable to enable stable transportation at ambient temperatures and long-time storage of reaction mixtures for point-of-care applications.

### SUMMARY

Described herein is a lyophilized composition for use in a nucleic acid amplification reaction. The reaction mixture can be used to test for a target nucleic acid in a sample, with the presence of the target nucleic acid in the sample assessed by simple visual detection of the presence or absence of a colorimetric change.

According to a first aspect, provided herein is a lyophilized composition, the composition comprising:
nucleic acid amplification reagents; a cryoprotectant; a pH adjusting agent; and a colorimetric amplification indicator. The composition can be provided for use in a nucleic acid amplification reaction. The composition can be provided to detect the presence of a target nucleic acid in a sample by performing a colorimetric nucleic acid amplification reaction.

In a second aspect, provided herein is a lyophilised composition for use as a color control composition in a colorimetric nucleic acid amplification reaction, wherein the composition corresponds to a composition of the first aspect, but with the exclusion of at least one nucleic acid amplification reagent. The composition may be absent nucleic acid polymerase. Where reverse transcriptase is present in a composition of the first aspect of the invention, this may be excluded in addition to, or instead of, exclusion of the nucleic acid polymerase.

In a third aspect, provided herein is a process for producing a lyophilized composition for use in a nucleic acid amplification reaction, the process comprising:
a. providing a pre-lyophilisation formulation comprising nucleic acid amplification reagents, a cryoprotectant, a pH adjusting agent and a colorimetric amplification indicator in water; and
b. lyophilizing the formulation to produce a lyophilized composition.

In a fourth aspect, provided herein is a process for producing a lyophilized composition for use as a color control composition in a colorimetric nucleic acid amplification reaction, the process corresponding to the process of the third aspect, but wherein nucleic acid polymerase is excluded from the pre-lyophilisation formulation. Where reverse transcriptase is present in a pre-lyophilisation formulation of the third aspect of the invention, this may be excluded in addition to, or instead of, exclusion of the nucleic acid polymerase.

In a fifth aspect, provided herein is a lyophilized composition produced by a process of the third or fourth aspect.

In a sixth aspect, provided herein is a rehydrated reaction mixture formed by rehydration of a lyophilised composition of any of the first, second or fifth aspects.

In a seventh aspect, provided herein is a method for determining the presence of a target nucleic acid in a test sample, the method comprising:
a. providing a lyophilised composition according to the first aspect in a first vessel;
b. rehydrating the lyophilised composition within the first vessel and adding the test sample to the first vessel to provide a rehydrated reaction mixture;
c. subjecting the rehydrated reaction mixture to conditions suitable to allow nucleic acid amplification to occur if the target nucleic acid is present in the test sample;
d. determining a positive result for the presence of a target nucleic acid in the test sample if a colorimetric change occurs due to the presence of nucleic acid amplification.

In an eighth aspect, provided herein is a method for diagnosing infection by a pathogen in a subject, the method comprising:
a. providing a lyophilised composition according to the first aspect, wherein the lyophilised composition contains primers directed to a target nucleic acid of the pathogen;
b. rehydrating the lyophilised composition and adding a test sample obtained from the subject to provide a rehydrated reaction mixture;
c. subjecting the rehydrated reaction mixture to conditions suitable to allow nucleic acid amplification to occur if the target nucleic acid is present in the test sample; and
d. determining a positive result for the presence of the target nucleic acid in the test sample if a colorimetric change occurs due to the presence of nucleic acid amplification; and
e. diagnosing infection by the pathogen on the basis of a positive result.

In a ninth aspect, provided herein is a method for treating infection by a pathogen in a subject, the method comprising:
a. providing a lyophilised composition according to the first aspect, wherein the lyophilised composition contains primers directed to a target nucleic acid of the pathogen;
b. rehydrating the lyophilised composition and adding a test sample from a subject to provide a rehydrated reaction mixture;
c. subjecting the rehydrated reaction mixture to conditions suitable to allow nucleic acid amplification to occur if the target nucleic acid is present in the sample;
d. determining a positive result for the presence of the target nucleic acid in the sample if a colorimetric change occurs due to the presence of nucleic acid amplification; and
e. diagnosing infection by the pathogen in the subject on the basis of a positive result and treating the subject for said infection.

In a tenth aspect, provided herein is a kit for carrying out a nucleic amplification reaction, the kit comprising a vessel having a lyophilised composition of the first aspect contained therein.

In an eleventh aspect, provided herein is a pre-lyophilisation formulation comprising nucleic acid amplification reagents; a cryoprotectant; a pH adjusting agent; a colorimetric amplification indicator; and water.

### BRIEF DESCRIPTION OF THE FIGURES

Embodiments of the disclosure will now be described, by way of example only, and with reference to the drawings in which:

FIG. 1 shows performance of wet and lyophilised LAMP reactions. (A) Analytical sensitivity of the lyophilised LAMP beads and the conventional LAMP protocol (liquid) using a serial 10-fold dilution of SARS-CoV-2 RNA and a tandem-LAMPcov assay. (B) Comparison of the ΔpH obtained after amplification of lyophilised LAMP beads and the conventional LAMP protocol (liquid) using a serial 10-fold dilution of SARS-CoV-2 RNA and a tandem-LAMPcov assay. A margin of error of 0.25 pH units is established as the threshold for negative reactions. In each pair of columns, the lyophilised reaction is the right hand column.

FIG. 2 shows the pH differential between pre and post lyophilization formulations and post-amplification. Pre-lyophilized mix (2× and 1×) pH compared to post-lyophilized cakes rehydrated in water (Rehydrated) and amplified at 63° C. for 30 minutes (Amplified). Both SARS-CoV-2 and Internal control (IC) assays were used.

FIG. 3 shows a representative result of a colorimetric LAMP reaction carried out as described here in. Tubes 1-8 contain, from left to right: color control (1), color control (2), color control (3), SARS-Cov-2 detection (containing positive patient sample) (4), SARS-Cov-2 detection (containing negative patient sample) (5), human control (6), internal control (7) and internal control (8). The color of the tubes is as indicated and demonstrates either a successful (yellow, positive result) or unsuccessful (pink, negative result) LAMP reaction.

FIG. 4 shows TTP (time to positive) results for various lyophilised LAMP reactions with mannitol and ficoll as cryoprotectants or trehalose and dextran as cryoprotectants.

### DETAILED DESCRIPTION

Described herein is a lyophilized composition for use in a nucleic acid amplification reaction. The reaction mixture can be used to test for a target nucleic acid in a sample, with the presence of the target nucleic acid in the sample assessed by simple visual detection of the presence or absence of a colorimetric change.

According to a first aspect, provided herein is a lyophilized composition, the composition comprising:
nucleic acid amplification reagents; a cryoprotectant; a pH adjusting agent; and a colorimetric amplification indicator.

The composition can be provided for use in a nucleic acid amplification reaction. The composition, when rehydrated, forms a medium (rehydrated reaction mixture) in which amplification of a nucleic acid may take place. The composition comprises components that enable amplification of a target nucleic acid to occur (nucleic acid amplification reagents).

Nucleic acid amplification can occur when the lyophilised composition is rehydrated and exposed to a sample, if the sample contains a target nucleic acid. The nucleic acid amplification reagents may comprise: a nucleic acid polymerase; a nucleotide triphosphate mixture; at least two primers directed to a target nucleic acid; and a source of a metal cofactor for the nucleic acid polymerase. The nucleic acid polymerase may be a DNA polymerase (for example a Bst polymerase, such as Bst 2.0 Warm Start). The DNA polymerase may be a glycerol free DNA polymerase (for example Bst 2.0 Warm Start glycerol free).

The target nucleic acid may be an RNA, DNA or cDNA. When the target nucleic acid is RNA, the nucleic acid amplification reagents may further comprise a reverse transcriptase (for example RTx Warm Start). The reverse transcriptase may be a glycerol free reverse transcriptase (for example RTx Warm Start glycerol free).

The target nucleic acid may be a nucleic acid sequence from a pathogen (such as a characteristic nucleic acid of a particular virus—for example, but not limited to, SARS-CoV-2, influenza, respiratory syncytial virus (RSV), human rhinovirus (HR), adenovirus or parainfluenza). In such embodiments, the composition may be used in a nucleic acid amplification reaction to determine the presence of a pathogen in a biological sample.

The target nucleic acid may be a nucleic acid sequence from a human gene. In such embodiments, the composition may be used in a nucleic acid amplification reaction to determine the presence of human DNA in a biological sample, for example to provide a control that a sample contains human DNA.

Metal ions are used as cofactors by nucleic acid polymerases. The source of a metal cofactor for the nucleic acid polymerase may be a metal salt, for example a divalent metal salt, for example a salt comprising Mg²⁺, Ca²⁺ or Mn²⁺ ions. The metal salt may be a magnesium salt, for example magnesium sulphate. It will be appreciated that the source of a metal cofactor should be selected to provide the appropriate metal ions suitable for use as a cofactor by the nucleic acid polymerase included in the composition.

The composition comprises components that enable amplification of a target nucleic acid to occur, along with a colorimetric amplification indicator that results in a color change of the reaction solution when nucleic acid amplification occurs. The composition may therefore be used to detect the presence of a target nucleic acid in a sample using colorimetric detection.

Detecting may be colorimetric detection. Colorimetric detection may be by the naked eye, or by use of a cell phone. Detecting may be colorimetric detection compatible with the naked eye or a cell phone. The cell phone may detect amplification using a camera and/or an app. The app may be an app designed for use with the method of the invention. The app may be supplied by a third party. The app may be software supplied with the cell phone. The app may be an image processing app. A phone or other suitable camera device may capture an image for detection. The image may then be processed on another device or on the same device.

During nucleic acid amplification, H⁺ ions are released. The accumulation of released H⁺ ions decreases the pH of a reaction solution. Metal ions such as Mg²⁺ are used as cofactors by the nucleic acid polymerase. As nucleic acid amplification occurs, a decrease in the concentration of free metal ions in the solution will occur. Accordingly, the colorimetric amplification indicator may be a pH sensitive indicator or a metal sensitive indicator, or a combination of one or more pH sensitive or metal sensitive indicators. For example, the colorimetric amplification indicator may be (a) a pH indicator selected from gentian violet, malachite green, thymol blue, methyl yellow, bromophenol blue, congo red, methyl orange, methyl orange bromocresol green, methyl red, methyl Purple, azolitmin red, bromocresol purple, bromothymol blue, phenol red, neutral red, naphtholphthalein, Cresol red, Cresolphthalein, Phenolphthalein, Thymolphthalein, Alizarine Yellow R yellow or Indigo carmine; (b) a metal sensitive indicator selected from hydroxynaphthol blue, eriochrome black t, calmagite, curcumin, fast sulphon black, hematoxylin, murexide, xylenon orange, BAPTA, BAPTA AM, BTC, BTC AM, Calcein, Calcein AM, Calcein Blue, Calcium Green 1, Calcium Green 2, Calcium Green 5N, Coelenterazine, Coelenterazine cp, Coelenterazine f, Coelenterazine h, Coelenterazine hep, Coelenterazine n, CoroNa Green, Corona Green AM, CoroNa Red, DAF FM, Fluo 3, Fluo 3 AM, PBFI AM, Phen Green SK, Quin 2, Quin 2 AM, RhodZin 3, hydroxynaphthol blue or calmagite; or (c) a combination of any two or more of these indicators. The indicator may be phenol red, thymol blue, Eriochrome Black T, or a combination thereof. The indicator may be phenol red. Phenol red may provide a colorimetric change from pink (negative) to yellow (positive). The composition may comprise both a pH sensitive indicator and a metal sensitive indicator, for example thymol blue and Eriochrome Black T (EBT). A combination of EBT and thymol blue may provide a colorimetric change from red (negative) to green (positive).

It is preferable that the solution formed by rehydration of a lyophilized composition as described herein is not pH buffered or has low pH buffering capacity, to facilitate detection of the pH change caused by nucleic acid amplification. Low-buffering capacity may refer to a buffering capacity not more than that provided by a 7.5 mM solution of Tris (tris(hydroxymethyl)aminomethane) buffer with a starting pH of 8.8. The low-buffering capacity may be not more than that provided by a 5, 2.5, 1, 0.75 or 0.5 mM solution of Tris (tris(hydroxymethyl)aminomethane) buffer with a starting pH of 8.8. The buffering capacity may be as measured at 25° C. The composition may be substantially free from Tris buffer or may be completely free from Tris buffer.

The composition comprises a pH adjusting agent. A pH adjusting agent is an agent that, when added to a solution, adjusts the pH of that solution. The pH adjusting agent may function to provide a lyophilised reaction composition that, when rehydrated, forms a solution with a suitable pH for performing a colorimetric nucleic amplification reaction, e.g. a pH where amplification reaction proceeds and where colorimetric detection can occur. The pH adjusting agent is preferably a pH raising agent. The pH adjusting agent may comprise a base or any other compound that provides an overall pH raising effect when dissolved in water. The pH adjusting agent may be a metal hydroxide (e.g. NaOH or KOH), ammonia, calcium carbonate, sodium acetate, potassium cyanide or sodium sulfide, or a mixture thereof. The pH adjusting agent is preferably NaOH or KOH, more preferably NaOH.

The composition comprises a cryoprotectant. The composition may comprise one cryoprotectant or more than one cryoprotectant. The presence of one or more cryoprotectants may benefit the lyophilisation process, for example acting to protect and stabilise proteins such as the nucleic acid polymerase or reverse transcriptase, when present. In addition, the inclusion of one or more cryoprotectant may enhance the kinetics of the nucleic acid amplification reaction.

A cryoprotectant may be a sugar reagent or a polyhydroxy reagent. A cryoprotectant may be a polysaccharide or polysaccharide-containing moiety. A cryoprotectant may be a surfactant. The surfactant may be a non-ionic surfactant. The surfactant may be a sugar-based or sugar alcohol-based surfactant. The cryoprotectant may be trehalose, dextran, mannitol, sucrose, sorbitol or a polymer of sucrose formed by copolymerisation of sucrose and epichlorohydrin (referred to herein as ficoll). The cryoprotectant may be trehalose, dextran, mannitol, or a polymer of sucrose formed by copolymerisation of sucrose and epichlorohydrin (referred to herein as ficoll). A combination of more than one of these cryoprotectants may be present. Dextran may be Dextran 70. Mannitol may be D-mannitol. Ficoll may be Ficoll 400. The composition may comprise a combination of mannitol and ficoll (preferably D-mannitol and Ficoll 400) or a combination of dextran and trehalose (preferably dextran 70 and trehalose).

The components of the composition may be present in the composition at relative amounts within the composition such that, on rehydration of the composition with water, a formulation with components at the following concentrations can be formed. In the composition, the pH adjusting agent may be present in an amount sufficient to produce a rehydrated reaction mixture with a target pH when the composition is rehydrated with water. The target pH may also be referred to as the target starting pH and is the pH of the rehydrated mixture immediately after rehydration and before a nucleic amplification reaction occurs. The target pH may be at least about 7.5, for example about 7.5-10 or about 7.5-9, preferably at least about 7.8 or at least about 8, for example about 7.8-9 or about 8-9. The target pH may be about 7.5-9, preferably about 7.5-8.5, more preferably about 7.8 to 8.5, 7.9 to 8.5 or 8 to 8.5. The pH adjusting agent and the source of a metal cofactor (e.g. MgSO₄) may be present in the composition at relative amounts sufficient such that, on rehydration with water, a formulation with a target pH as mentioned above, and a concentration of the source of a metal cofactor (e.g. MgSO₄) that is tolerable for activity of the nucleic acid polymerase, can be formed. The pH adjusting agent may be present in an amount sufficient to produce a rehydrated mixture with a target pH as mentioned above, when rehydrated with a volume of water sufficient to provide a concentration of the source of a metal cofactor (e.g. MgSO₄) of about 4-20 mM, 4-15 mM, 4-10 mM, 4-8 mM, 6-8 mM, 4-7 mM or 6-7 mM in the rehydrated mixture. It is to be understood that this requirement is met if a pH within the target range is reached when a composition is rehydrated by a sufficient amount of water to give a concentration of the source of a metal cofactor in the rehydrated mixture corresponding to at least one value within the range of about 4-10 mM. It is not necessary for the target pH to be met when a composition is rehydrated to all possible values within the target pH range. For example, a composition giving a target pH when rehydrated to provide a source of a metal cofactor concentration of 8 mM would meet this requirement even if the same composition were to provide a pH outside this target range if diluted further to give a source of a metal cofactor concentration of 4 mM.

The components of the composition may be present in the composition at relative amounts within the composition such that, on rehydration with water, a rehydrated formulation with components at the following concentrations can be formed.

The composition may be capable of being rehydrated to provide a rehydrated formulation comprising:
the source of a metal cofactor (e.g. MgSO₄) at a concentration of about 4-20 mM, 4-15 mM, 4-10 mM, 4-8 mM, 6-8 mM, 4-7 mM or 6-7 mM; and
the pH adjusting agent (e.g. NaOH) at a concentration sufficient to provide a target pH as recited above, for example a pH of about 7.5-9, 7.5-8.5, or 7.8 to 8.5.

The composition may be capable of being rehydrated to provide a rehydrated formulation comprising:
the source of a metal cofactor (e.g. MgSO₄) at a concentration of about 4-20 mM, 4-15 mM, 4-10 mM, 4-8 mM, 6-8 mM, 4-7 mM or 6-7 mM; and
the pH adjusting agent (e.g. NaOH) at a concentration of at least about 5 mM, at least about 6 mM, at least about 7 mM, at least about 8 mM, at least about 8.5 mM, or at least about 8.75 mM. The pH adjusting agent in the rehydrated formulation may be at a concentration of no more than about 20 mM, no more than about 15 mM, preferably no more than about 14 mM, preferably at a concentration of about 5-20 (e.g. 8.75-14) nM.

The composition may further be capable of being rehydrated to provide a rehydrated formulation comprising a cryoprotectant concentration of at least about 0.5% w/v, at least about 1% w/v or at least about 1.5% w/v. The composition may further be capable of being rehydrated to provide a rehydrated formulation comprising a cryoprotectant concentration of up to about 20% w/v, up to about 15% w/v or up to about 10% w/v. The composition may be capable of being rehydrated to provide a rehydrated formulation comprising a cryoprotectant concentration of about 1-10 or 1-5% w/v. The cryoprotectant concentration may be the total cryoprotectant concentration if more than one cryoprotectant is present. The composition may further be capable of being rehydrated to provide a rehydrated formulation comprising at least one of: a) a mannitol concentration of about 1-10% w/v, 1-5% w/v, 1-4% w/v, 1-3% w/v or 2% w/v b) a Ficoll concentration of about 0.5-5% w/v, 0.5-3% w/v or 0.5-2% w/v, 0.5-1.5% w/v or 1% w/v; c) a trehalose concentration of about 1-10% w/v, 1-5% w/v, 1-4% w/v, 1-3% w/v or 2% w/v; and d) a dextran concentration of 0.5-10% w/v, 0.5-5% w/v, 0.5-4% w/v, 1-3% w/v or 2% w/v. Preferably, the composition comprises mannitol and ficoll, or dextran and trehalose, at relative amounts suitable to provide the above compositions in a rehydrated formulation.

The composition may be capable of being rehydrated to provide a rehydrated formulation comprising the pH adjusting agent (e.g. NaOH) at a concentration sufficient to provide a target pH as recited above, for example a pH of about 7.5-9, 7.5-8.5, or 7.8 to 8.5 and with a cryoprotectant concentration as described above.

The composition may be capable of being rehydrated to provide a rehydrated formulation comprising:
the pH adjusting agent (e.g. NaOH) at. a concentration of at least about 5 mM, at least about 6 mM, at least about 7 mM, at least about 8 mM, at least about 8.5 mM, or at least about 8.75 mM, and with a cryoprotectant concentration as described above. The composition may be capable of being rehydrated to provide a rehydrated formulation comprising: the pH adjusting agent (e.g. NaOH) at. a concentration of about 5-20 (e.g. 8.75-14) nM, and with a cryoprotectant concentration as described above.

The composition may further be capable of being rehydrated to provide a rehydrated formulation comprising the colorimetric amplification indicator at a concentration of at least about 50 μM, for example about 50-300 μM. The indicator concentration may be the total indicator concentration if more than one indicator is present. The composition may further be capable of being rehydrated to provide a rehydrated formulation comprising: a) phenol red at a concentration of about 50-100 μM; or b) thymol blue at a concentration of about 50-175 μM and EBT at a concentration of about 50-100 μM.

The composition may further be capable of being rehydrated to provide a rehydrated formulation comprising:
the nucleic acid polymerase at a concentration of at least about 0.3 U/μL, 0.3-2 U/μL, 0.3-1 U/μL or 0.3-0.7 U/μL; and/or
the reverse transcriptase enzyme, if present, at a concentration of at least about 0.15 U/μL, a concentration of about 0.15-5 U/μL, or a concentration of about 0.15-3.25 U/μL; and/or
the nucleotide triphosphate mixture at a concentration or at least about 1 mM, from about 1 to 2 mM or about 1.2 to 1.7 mM.

It should be appreciated that throughout this disclosure, wherever discussion of the relative components of the composition is provided, these components can be present in any combination and the concentrations capable of being provided by the composition on rehydration may be present in any combination.

The composition may further comprise an enzyme stabiliser. The enzyme stabiliser may be bovine serum albumin (BSA), DMSO, gelatin, Prionex (a polypeptide fraction of highly purified dermal collagen of porcine origin), sodium alginate, or a combination thereof. The enzyme stabiliser is preferably BSA.

The composition may further comprise at least one additive, selected from Triton X-100, KCl, (NH₄)₂SO₄, Tween 20 (polyoxyethylene (20) sorbitan monolaurate), sodium azide, spermidine, tergitol, Brij-35, DMSO, formamide, PEG 35K, PEG 8K, GuHCl, GuTC, dithiothreitol (DTT) and ethylenediaminetetraacetic acid (EDTA).

The composition may further be capable of being rehydrated to provide a rehydrated formulation comprising an enzyme stabilizer (e.g. BSA) at a concentration of about 0-10 mg/mL, 0-5 mg/mL, 0.5-10 mg/mL, 0.5-5 mg/mL, 1-10 mg/mL, 1-5 mg/mL, 1-3 mg/mL or 1-2 mg/mL.

The composition may further be capable of being rehydrated to provide a rehydrated formulation comprising one or more of:

| Component | Concentration |
|---|---|
| Triton X-100 | about 0-5%, 0-3%, or 0-2% |
| Tween 20 | about 0-2%, or 0.1-1.8% |
| Tergitol | about 0-5%, 0-3%, 0-2%, or 0-1.5% |
| Brij-35 | about 0-10%, 0-5%, or 0-4% |
| DMSO | about 0-4% |
| Formamide | about 0-5%, 0-4%, or 0-2% |
| Sodium Azide | about 0-0.04% |
| Spermidine | about 0-0.1% |
| Polyethyleneglycol | about 0-7% |
| 35K | |
| Polyethyleneglycol | about 0-10% |
| 8K | |
| GuHCl | about 0-40 mM |
| GuTC | about 0-20 mM |
| Glycerol | about 0-2%, 0-1%, 0-0.5% |
| KCl | about 0-110 mM, 0-100 mM, 0-70 mM, |
| | 30-70 mM, or 40-60 mM |
| (NH₄)₂SO₄ | about 0-20 mM or 5-15 mM |

wherein all percentages are % w/v (with reference to the volume of rehydrated formulation).

The composition may be capable of being rehydrated to provide a rehydrated formulation with a low-buffering capacity, for example with a buffering capacity of no more than that provided by a 7.5 mM solution of Tris buffer with a starting pH of 8.8. The composition may be capable of being rehydrated to provide a rehydrated formulation with a buffering capacity of no more than that provided by a 5 mM solution of Tris buffer with a starting pH of 8.8 or a 2.5 mM solution of Tris buffer with a starting pH of 8.8. The composition may be capable of being rehydrated to provide a rehydrated formulation with a buffering capacity of no more than that provided by a 1 mM solution of Tris buffer with a starting pH of 8.8, a 0.75 mM solution of Tris buffer with a starting pH of 8.8 or a 0.5 mM solution of Tris buffer with a starting pH of 8.8.

Accordingly, the composition may be capable of being rehydrated to provide a rehydrated formulation with a buffering capacity equivalent to Tris buffer at a concentration of about 0-7.5 mM, 0-5 mM, 0-2.5 mM, 0-1 mM, 0.0.75 mM or 0-0.5 mM in a solution having a starting pH of 8.8. These buffering capacities may be measured when the composition is rehydrated with water sufficient to provide the source of a metal cofactor (e.g. MgSO₄) at a concentration as described herein (e.g. of about 4-10 mM). These buffering capacities may be measured when the composition is rehydrated with water sufficient to provide a cryoprotectant concentration as described above. The buffering capacity may be as measured at 25° C. It is not necessary for the target buffering capacity to be met when a composition is rehydrated to all possible values within the source of a metal cofactor concentration range. For example, a composition giving a target buffering capacity when rehydrated to provide a source of a metal cofactor concentration of 4 mM would meet this requirement even if the same composition were to provide a buffering capacity outside this target range when diluted less to give a source of a metal cofactor concentration of 10 mM.

The composition may further be capable of being rehydrated to provide a rehydrated formulation comprising Tris buffer (e.g. TrisHCl) at a concentration of no more than about 7.5 mM, no more than about 2.5 mM, no more than about 1 mM, no more than about 0.75 mM or no more than about 0.5 mM, or a formulation absent Tris buffer.

The composition may be substantially free of glycerol. The composition may be capable of being rehydrated to provide a rehydrated formulation with a glycerol concentration of no more than about 2% w/v, no more than about 1% w/v or no more than about 0.5% w/v, when the composition is rehydrated with water. The composition may be capable of being rehydrated to provide a rehydrated formulation with a glycerol concentration of no more than about 2% w/v, no more than about 1% w/v or no more than about 0.5% w/v, when the composition is rehydrated with water such to produce a rehydrated reaction mixture with a target pH as recited above, for example of about 7.5-9, 7.5-8.5, or 7.8 to 8.5. The composition may be capable of being rehydrated to provide a rehydrated formulation with a glycerol concentration of no more than about 2% w/v, no more than about 1% w/v or no more than about 0.5% w/v, when the composition is rehydrated with water sufficient to provide the source of a metal cofactor (e.g. MgSO₄) at a concentration as described herein (e.g. of about 4-10 mM). The composition may be capable of being rehydrated to provide a rehydrated formulation with a glycerol concentration of no more than about 2% w/v, no more than about 1% w/v or no more than about 0.5% w/v, when the composition is rehydrated with water sufficient to provide any other component at a concentration range as recited herein for the rehydrated composition, for example a cryoprotectant concentration as described above. It is not necessary for the target glycerol concentration to be met when a composition is rehydrated to all possible values within the target pH range, the source of a metal cofactor concentration range or the concentration range of any other component. For example, a composition giving a target glycerol concentration when rehydrated to provide a source of a metal cofactor concentration of 4 mM would meet this requirement even if the same composition were to provide a buffering capacity outside this target range when diluted less to give a source of a metal cofactor concentration of 10 mM. Reference to substantially free of glycerol includes free of glycerol. Accordingly, the composition may be free of glycerol. For example, all components of the composition may be glycerol-free components.

The composition may further comprise an internal control template nucleic acid. The internal control template nucleic acid may be a nucleic acid of non-human origin. The internal control template nucleic acid may be a synthetic nucleic acid, for example comprising a sequence based on a non-human animal gene. The internal control template nucleic acid sequence may be a synthetic non-biological sequence, i.e. fully artificial and not based on any natural gene. In a composition comprising an internal control template nucleic acid, the internal control template nucleic acid may be the target nucleic acid and the composition may comprise primers directed to the internal control template nucleic acid sequence. The composition may not comprise primers directed against any other target nucleic acid.

A composition comprising an internal control template nucleic acid contains primers and their respective template DNA. This can be used in a nucleic acid amplification reaction to check for correct storage of reagents, that appropriate conditions are used for the amplification reaction to occur and that a test sample is free from significant inhibitory contaminants. If all three conditions are satisfied, nucleic acid amplification will occur giving rise to a colorimetric change, and a test result can be confirmed as valid.

The lyophilised composition may be a composition obtainable by lyophilisation of a pre-lyophilisation formulation comprising the nucleic acid amplification reagents; cryoprotectant; pH adjusting agent; colorimetric amplification indicator, and water.

The water may comprise nuclease-free water.

The components of the pre-lyophilisation formulation may be solubilised in the water.

The pre-lyophilisation formulation may comprise the pH adjusting agent (e.g. NaOH) and the source of a metal cofactor (e.g. MgSO₄) present at a molar ratio of at least 1:1 (pH adjusting agent:cofactor). The pH adjusting agent may be present in a molar excess compared to the source of metal cofactor. The pH adjusting agent:cofactor molar ratio may be at least 1.05:1.

The pre-lyophilisation formulation may comprise the source of a metal cofactor (e.g. MgSO₄) at a concentration of about 8-40 mM, 8-30 mM, 8-20 mM, 4-16 mM, 12-20 mM, 8-14 mM or 12-14 mM.

The pre-lyophilisation formulation may comprise the pH adjusting agent (e.g. NaOH) at a concentration of at least about 10 mM, at least about 12 mM, at least about 14 mM, at least about 16 mM, at least about 17 mM, at least about 18 mM, or at least about 20 mM. The pH adjusting agent may be at a concentration of no more than about 40 mM, no more than about 30 mM, preferably no more than about 27 mM. The pH adjusting agent may be at a concentration of about 10-40 nM or 17.5-28 mM. The pH adjusting agent may preferably be at a concentration of about 17.5-27 mM, 19-27 mM or 20-27 mM.

The pre-lyophilisation formulation may comprise a cryoprotectant concentration of at least about 1% w/v, at least about 2% w/v or at least about 3% w/v. The cryoprotectant concentration may be up to 40% w/v, up to 30% w/v or up to about 20% w/v. The pre-lyophilisation formulation may comprise a cryoprotectant concentration of about 2-20 or 2-10% w/v. The cryoprotectant concentration may be the total cryoprotectant concentration if more than one cryoprotectant is present. The pre-lyophilisation formulation may comprise at least one of: a) a mannitol concentration of about 2-20% w/v, 2-10% w/v, 2-8% w/v, 2-6% w/v or 4% w/v; b) a Ficoll concentration of about 1-10% w/v, 1-6% w/v, 1-4% w/v, 1-3% w/v or about 2% w/v; c) a trehalose concentration of about 2-20% w/v, 2-10% w/v, 2-8% w/v, 2-6% w/v or about 4% w/v; and d) a dextran concentration of about 1-20% w/v, 1-10% w/v, 1-8% w/v, 2-6% w/v or about 4% w/v. Preferably, the pre-lyophilisation formulation comprises mannitol and ficoll, or dextran and trehalose, at concentrations as mentioned above.

The pre-lyophilisation formulation may comprise the colorimetric amplification indicator at a concentration of at least about 100 μM, for example about 100-600 μM. The indicator concentration may be the total indicator concentration if more than one indicator is present. The pre-lyophilisation formulation may comprise: a) phenol red at a concentration of about 100-200 μM; or b) thymol blue at a concentration of about 100-350 μM and EBT at a concentration of about 100-200 μM.

The pre-lyophilisation formulation may comprise:
the nucleic acid polymerase at a concentration of at least about 0.6 U/μL, or about 0.6-4 U/μL, 0.6-2 U/μL, 0.6-1.4 U/μL or 1 U/μL; and/or
the reverse transcriptase enzyme, if present, at a concentration of at least about 0.3 U/μL, a concentration of about 0.3-10 U/μL, a concentration of about 0.3-6.5 U/μL, or a concentration of about 0.9 U/μL; and/or
the nucleotide triphosphate mixture at a concentration of a least about 2 mM, or about 2 to 4 mM, 2.4 to 3.4 mM or 2.8 mM.

It should be appreciated that throughout this disclosure, wherever discussion of the concentrations of the components of the pre-lyophilisation formulation is provided, these components can be present in any combination and the concentrations may be present in any combination.

The pre-lyophilisation formulation may further comprise an enzyme stabiliser. The enzyme stabiliser may be bovine serum albumin (BSA), DMSO, gelatin, Prionex (a polypeptide fraction of highly purified dermal collagen of porcine origin), sodium alginate, or a combination thereof. The enzyme stabiliser is preferably BSA.

The pre-lyophilisation formulation may further comprise at least one additive, selected from Triton X-100, KCl, (NH₄)₂SO₄, Tween 20 (polyoxyethylene (20) sorbitan monolaurate), sodium azide, spermidine, tergitol, Brij-35, DMSO, formamide, PEG 35K, PEG 8K, GuHCl, GuTC, dithiothreitol (DTT) and ethylenediaminetetraacetic acid (EDTA).

The pre-lyophilisation formulation may further comprise an enzyme stabilizer (e.g. BSA) at a concentration of about 0-20 mg/mL, 0-10 mg/mL, 1-20 mg/mL, 1-10 mg/mL, 2-20 mg/mL, 2-10 mg/mL, 2-6 mg/mL, 2-4 mg/mL or 2.4 mg/mL.

The pre-lyophilisation formulation may comprise one or more of:

| Component | Concentration |
|---|---|
| Triton X-100 | about 0-10%, 0-6%, or 0-4% |
| Tween 20 | about 0-4%, or 0.2-3.6% |
| Tergitol | about 0-10%, 0-6%, 0-4%, or 0-3% |
| Brij-35 | about 0-20%, 0-10%, or 0-8% |
| DMSO | about 0-8% |
| Formamide | about 0-10%, 0-8%, or 0-4% |
| Sodium Azide | about 0-0.08% |
| Spermidine | about 0-0.2% |
| Polyethyleneglycol | about 0-14% |
| 35K | |
| Polyethyleneglycol | about 0-20% |
| 8K | |
| GuHCl | about 0-80 mM |
| GuTC | about 0-40 mM |
| Glycerol | about 0-4%, 0-2%, 0-1% |
| KCl | about 0-220 mM, 0-200 mM, 0-140 mM, |
| | 60-140 mM, or 80-120 mM |
| (NH₄)₂SO₄ | about 0-40 mM or 10-30 mM |

wherein all percentages are % w/v.

The pre-lyophilisation formulation may be substantially free of glycerol. The pre-lyophilisation formulation may have a glycerol concentration of no more than about 4% w/v, no more than about 2% w/v or no more than about 1% w/v. The pre-lyophilisation formulation composition may be free of glycerol.

The pre-lyophilisation formulation may further comprise Tris buffer (e.g. TrisHCl) at a concentration of about 0-15 mM, 0-5 mM, 0-2 mM, 0-1.5 mM or 0-1 mM. The pre-lyophilisation formulation may be free of Tris buffer.

It will be appreciated that the pre-lyophilisation formulation may be provided in a further diluted or a more concentrated form. Accordingly, a formulation comprising the same components of any embodiments of the pre-lyophilisation formulation described herein, at corresponding relative concentrations, may also be provided. For example, the pre-lyophilisation formulation described herein may represent a 2× formulation, i.e. a formulation that, on rehydration, will be rehydrated to double the volume of the pre-lyophilisation formulation. A 1× formulation may also be provided wherein, on rehydration, the formulation is rehydrated to the same volume as the pre-lyophilisation formulation. For such a formulation, the concentrations of all components provided herein are halved (i.e. reduced by a factor of 2). It will be appreciated that any factor of concentration or dilution may apply, provided appropriate relative concentrations are maintained.

A loss of pH may occur during the lyophilisation process. The composition of the pre-lyophilisation formulation, e.g. the amount of pH adjusting agent, may be determined to account for this pH loss such that, despite the pH loss, the target pH of the rehydrated mixture is met. Accordingly, the pre-lyophilisation mixture may be formulated to have a pH higher than the target pH of the rehydrated mixture. The pre-lyophilisation formulation may have a pH of at least about 8.5, for example about 8.5-12, about 8.5-11, or about 8.5-10.5, preferably at least about 9 or at least about 9.5, for example about 9.5-10.5.

Benefits of the provision of a lyophilized composition for use in a colorimetric nucleic acid amplification reaction include enabling stable transportation and storage at ambient temperatures.

In a second aspect, provided herein is a lyophilised composition for use as a color control composition in a colorimetric nucleic acid amplification reaction, wherein the composition corresponds to a composition of the first aspect, but with the exclusion of at least one component to prevent nucleic acid amplification even in the presence of environmental or other contamination. The lyophilised composition for use as a color control composition in a colorimetric nucleic acid amplification reaction, may be a composition that corresponds to a composition of the first aspect, but with the exclusion of at least one nucleic acid amplification reagent. The composition may be absent nucleic acid polymerase or primers. The composition may be absent nucleic acid polymerase. Where reverse transcriptase is present in a composition of the first aspect of the invention, this may be excluded in addition to, or instead of, exclusion of the nucleic acid polymerase or primers.

All features described in relation to the first aspect may apply to the second aspect mutatis mutandis.

In a third aspect, provided herein is a process for producing a lyophilized composition for use in a nucleic acid amplification reaction, the process comprising:
a. providing a pre-lyophilisation formulation comprising nucleic acid amplification reagents, a cryoprotectant, a pH adjusting agent, a colorimetric amplification indicator and water; and
b. lyophilizing the formulation to produce a lyophilized composition.

The pre-lyophilisation formulation may be as described in relation to the first aspect. The pre-lyophilisation formulation may be provided as a solution in water.

Lyophilization (also referred to as freeze-drying) of the pre-lyophilisation formulation may be performed using a flash freeze-drying process or a ramp freeze-drying process. The freeze-drying process may comprise an annealing stage.

The freeze-drying process may be a flash freeze-drying process, optionally comprising an annealing stage.

The freeze-drying process may comprise initial freezing of the pre-lyophilisation formulation to a freeze temperature. The freeze temperature may be about −40° C. or lower, about −45° C. or lower or about −50° C. or lower. The freeze temperature may be no lower than about −80° C., no lower than about −70° C. or no lower than about −60° C. The freeze temperature may be below the collapse temperature (Tc) of the formulation. The freeze temperature may be within about 15° C., 10° C. or 5° C. of the collapse temperature (Tc) of the formulation. The initial freezing may be carried out as flash-freeze or a ramp-freeze. Preferably this is carried out as a flash freeze, where the pre-lyophilisation formulation is loaded to a freeze-dryer already cooled to the freeze temperature.

If ramp-freezing is used, the temperature may be ramped to the freeze temperature, over a time period. The time period may be at least about 1 hour, for example a time period of about 1-4 hours, 2-3 hours or 2 hours.

If flash-freezing is used, in the initial freezing step, the pre-lyophilisation formulation may be loaded to a freeze dryer already at the freeze temperature and held at that temperature for a time period. The time period may be at least about 1 hour, for example a time period of about 1-4 hours, 2-4 hours or 3 hours. Alternatively, the time period may be at least about 1 hour, for example a time period of about 1-4 hours, 1-3 hours or 2 hours.

An annealing stage may be present after initial freezing. Annealing may comprise increasing the temperature to an increased temperature of about −10° C. to −20° C., preferably about −15° C., and decreasing the temperature back to a freeze temperature. If an annealing stage is present, this may comprise, increasing the temperature to a temperature of about −10° C. to −20° C., preferably about −15° C., over a time period of about 0.5-2 or 1-1.5 hours. The temperature may be held at the increased temperature, for example for a time period of about 1-3 or 2 hours. The temperate may then be decreased to the freeze temperature over a period of about 0.5-2 or 1-1.5 hours. The temperature may then be held at the freeze temperature for a time period of about 0.5-2 or 1 hours

Initial freezing may be carried out at atmospheric pressure. Annealing may be carried out at atmospheric pressure.

A primary drying stage may occur after initial freezing and after annealing, if present. Primary drying may be carried out at a freeze temperature and at a reduced pressure of no more than about 3000 μBar, no more than about 1000 μBar, no more than about 100 μBar, for example about 10-80 μBar, or 20-60 μBar. The freeze temperature may be below the collapse temperature (Tc) of the formulation. The freeze temperature may be about −35° C. or lower, about −40° C. or lower, about −45° C. or lower or about −50° C. or lower, preferably about −40° C. or lower, about −45° C. or lower or about −50° C. or lower. The freeze temperature may be no lower than about −70° C. or no lower than about −60° C. The freeze temperature may be the same as the freeze temperature of the initial freezing, or within about 10° C. or 5° C. of the freeze temperature of the initial freezing.

Primary drying may be carried out for a time period before secondary drying. The time period may be at least about 10 hours, at least about 20 hours, at least about 30 hours or at least 40 hours, for example a time period of about 20-100 hours, 40-80 hours or 40-60 hours.

Primary drying may also be carried out at a freeze temperature and at a reduced pressure of no more than about 3000 μBar, no more than about 1000 μBar, no more than about 200 μBar, for example about 10-200 μBar, or about 150-200 μBar (e.g. about 180 μBar). The freeze temperature may be below the collapse temperature (Tc) of the formulation. The freeze temperature may be about −25° C. or lower or about −30° C. or lower. The freeze temperature may be no lower than about −70° C. or no lower than −60° C. The freeze temperature may be the same as the freeze temperature of the initial freezing, or within about 15° C., 10° C. or 5° C. of the freeze temperature of the initial freezing. Primary drying may be carried out at a first freeze temperature, followed by lowering to a second freeze temperature. Primary drying may comprise an initial hold at a first freeze temperature that is the same as the freeze temperature of the initial freezing, or within about 10° C. or 5° C. of the freeze temperature of the initial freezing, followed by ramping to and holding at a second freeze temperature 5-10° C. lower than the first freeze temperature, optionally wherein the first freeze temperature is −35 to −45° C. and the second freeze temperature is −25 to −35° C. Primary drying may carried out for a time period before secondary drying of at least about 6 hours, at least about 10 hours, for example a time period of about 10-15 hours.

A secondary drying stage may occur after primary drying. Secondary drying may be carried out a temperature of about 0° C. or higher, for example about 0-30° C., 10-30° C., 15-25° C. or 20° C. Secondary drying may involve a ramp step, where the temperature is increased from freeze temperature of primary drying to the secondary drying temperature, followed by a hold step, held at the secondary drying temperature. The ramp step may be for a time period of least about 0.5 hours, at least about 1 hour, for example a time period of about 0.5-4 hours, 1-4 hours or 1-2 hours. The hold step may be for a time period of at least about 2 hours, at least about 4 hours, for example a time period of about 2-10 hours, 4-8 hours, 5-7 hours or 6 hours. Secondary drying may be carried out a pressure of no more than about 3000 μBar, no more than about 1000 μBar, no more than about 100 μBar, for example about 10-80 μBar, or 20-60 μBar. Secondary drying may also be carried out a pressure of no more than about 3000 μBar, no more than about 1000 μBar, no more than about 200 μBar, for example about 10-200 μBar, or about 150-200 μBar (e.g. about 180 μBar). The pressure may be the same as the pressure used in primary drying.

After secondary drying, a step of aeration with an inert gas, e.g. N₂ gas, may be carried out. This may be carried out a temperature of about 0° C. or higher, for example about 0-30° C., 10-30° C., 15-25° C. or about 20° C. Aeration may be carried out at the same temperature as secondary drying. Aeration may be carried out at atmospheric pressure.

It will be appreciated that the pre-lyophilisation formulation may comprise any of the components as described in respect of the composition of the first aspect and that all features described in relation to the first aspect may apply to the second aspect mutatis mutandis.

The pre-lyophilisation formulation may be provided in a vessel, e.g a sample tube, such that lyophilisation provides a lyophilised composition contained with the vessel, e.g. sample tube.

The lyophilised composition may be packaged and stored under an inert gas environment. After lyophilization, preferably immediately after lyophilization, the lyophilised composition may be transferred into an inert gas environment for packaging. The inert gas may be argon (Ar), helium (He), neon (Ne), krypton (Kr), xenon (Xe), and radon (Rn), preferably argon (Ar). Preferably the inert gas is not nitrogen gas (N₂). Packaging may comprise closing the vessel in which the lyophilized composition has been formed, or placing the lyophilized composition in a vessel and closing it, under an inert gas. Optionally the closed vessel may be placed inside a secondary packaging. This secondary packaging can, for example, be a heat-sealable foil pouch. Before heat-sealing the pouch, desiccant may be placed in the pouch (or other secondary packaging). This entire process, including sealing or heat-sealing the secondary packaging, may be carried out in the inert gas environment.

In a fourth aspect, provided herein is a process for producing a lyophilized composition for use as a color control composition in a colorimetric nucleic acid amplification reaction, the process corresponding to the process of the third aspect, but wherein a nucleic acid amplification reagent (e.g. nucleic acid polymerase) is excluded from the pre-lyophilisation formulation. Where reverse transcriptase is present in a pre-lyophilisation formulation of the third aspect of the invention, this may be excluded in addition to, or instead of, exclusion of the nucleic acid polymerase.

It will be appreciated that all features described in relation to pre-lyophilisation formulation of the first and second aspects may apply to the fourth aspect mutatis mutandis.

Lyophilisation in a process of the fourth aspect may be as described for the third aspect. All features described in relation to process of the third aspect may apply to the fourth aspect mutatis mutandis.

In a fifth aspect, provided herein is a lyophilized composition produced by a process of the third or fourth aspect.

In a sixth aspect, provided herein is a rehydrated reaction mixture formed by rehydration of a lyophilised composition of any of the first, second or fifth aspects.

In a seventh aspect, provided herein is a method for determining the presence of a target nucleic acid in a test sample, the method comprising:
a. providing a lyophilised composition according to the first aspect in a first vessel;
b. rehydrating the lyophilised composition within the first vessel and adding the test sample to the first vessel to provide a rehydrated reaction mixture;
c. subjecting the rehydrated reaction mixture to conditions suitable to allow nucleic acid amplification to occur if the target nucleic acid is present in the test sample;
d. determining a positive result for the presence of a target nucleic acid in the test sample if a colorimetric change occurs due to the presence of nucleic acid amplification.

The test sample may be any suitable sample containing a nucleic acid or suspected of containing a nucleic acid. The sample may comprise an eluent. This may be an aqueous eluent, for example water (e.g. nuclease free water). The test sample may comprise extracted nucleic acids (RNA and/or DNA) from any type of biological or environmental sample. Alternatively, a biological or environmental sample may be used directly as the test sample, without extraction of nucleic acid. A biological sample may be, for example, a nasopharyngeal swab, or a blood, a urine, a saliva, a tissue, stool, serum, lymph, semen, sweat, tears, amniotic fluid or wound exudate sample, or any other bodily fluid. An environmental sample may be, for example, water, such as waste water. It will be appreciated that nucleic acids may be extracted from a biological or environmental sample using any suitable technique, for example involving lysis and elution into a sample eluent to provide the test sample.

A colorimetric change occurs due when nucleic acid amplification occurs due to the presence of a colorimetric indicator. This may give a colorimetric change as a result of the change in pH or metal ion concentration cases by nucleic acid amplification. The colorimetric change may be detected by the naked eye, or by use of a cell phone or other suitable camera device. The cell phone may detect amplification using a camera and/or an app. The app may be an image processing app. A phone or other suitable camera device may capture an image for detection. The image may then be processed on another device or on the same device.

The target nucleic acid may be a nucleic acid sequence from a pathogen, for example SARS-CoV-2, influenza, respiratory syncytial virus (RSV), human rhinovirus (HR), adenovirus or parainfluenza. The method may therefore allow determination of the presence of a pathogen in a test sample, such as a test sample from a biological sample, for example from a subject suspected to be infected by the pathogen.

Subjecting the rehydrated reaction mixture to conditions suitable for to allow nucleic acid amplification to occur may comprise heating the contents of the vessel (the rehydrated reaction mixture and test sample) to a temperature suitable to achieve nucleic acid amplification, i.e. at which the nucleic acid polymerase enzyme is active. The temperature selected may be from about 30-95° C., 50-80° C., 60-72° C. 60-65° C., or 62-64° C. For example, the temperature may be about 63° C.

Any duration of the amplification step (step c) may be combined with any reaction temperature as defined above as long as the reaction conditions allow for amplification to occur and an amplification product can be detected. The temperature and duration may be optimised by comparison with a positive and/or a negative control. For example, a reaction temperature and duration may be selected to allow for detectable amplification of product in a positive control and/or with the nucleic acid from the sample but not to allow for detectable amplification in another sample and/or a negative control.

The duration of the amplifying step may be about 1 to 150 minutes, at least 15 minutes, at least 20 minutes, preferably 20-40 minutes or 20-30 minutes. Accordingly, heating may occur for about 1 to 150 minutes, at least 15 minutes, at least 20 minutes, preferably 20-40 minutes or 20-30 minutes. This causes the target nucleic acid, if present, to be amplified, thereby causing a color change. If the target nucleic acid is not present, no amplification will take place, and consequently the color of the rehydrated reaction mixture will not change

The nucleic acid amplification reaction may be isothermal nucleic acid amplification method, preferably Loop-Mediated Isothermal Amplification (LAMP). Nucleic acid amplification occurs under isothermal conditions. Accordingly, subjecting the rehydrated reaction mixture to conditions suitable to allow nucleic acid amplification to occur may comprise a step of holding the reaction mixture under isothermal conditions. The isothermal conditions may be any suitable conditions for nucleic acid amplification without the need for cycling between different temperatures for different steps in the amplification reaction, in contrast to traditional polymerase chain reaction (PCR). Isothermal conditions may be maintained using a laboratory water bath or heat block. The temperature and duration of amplification may be as described above.

Rehydration of the lyophilised composition and addition of the test sample may be carried out as a single step, by addition of a liquid test sample to the lyophilised composition. Alternatively, rehydration may occur by adding water and separately adding a test sample.

The method may be used to determine the presence of more than one target nucleic acid, for example from more than one pathogen. In such a method, more than one first vessel may be present, each vessel containing a lyophilised composition of the first aspect directed to a different target nucleic acid. This enables a panel of tests to be run, determining the presence or absence of more than one target nucleic acid in a test sample. Accordingly, the method therefore enables a test sample, for example from a biological sample from a patient, to be tested for the presence of multiple pathogens.

The method may further involve use of a lyophilised composition according to the second aspect to provide a color control in the method. This composition may be exposed to test sample and subjected to the same conditions as the composition of the first aspect. Due to the absence of at least one component (e.g. at least one nucleic acid amplification reagent) in the lyophilised composition of the second aspect, nucleic acid amplification will not occur, irrespective of the presence of target nucleic acid. Accordingly, the color change associated with a positive result for the presence of target nucleic acid should not occur in this color control and the color control may be used as a negative control and reference to which other reactions may be compared.

The method may therefore further comprise the steps of
a′. providing a lyophilised composition according to the second aspect in a second vessel;
b′. rehydrating the lyophilised composition within the second vessel and adding test sample to the second vessel to provide a rehydrated reaction mixture; and
c′. subjecting the rehydrated reaction mixture to the same conditions as in step c.

The method may further comprise comparing the color of the reaction mixture in the first and second vessels after steps c and c′ and confirming the presence of a target nucleic acid in the test sample if a colorimetric change occurs in the first vessel but not in the second vessel.

The method may further involve use of a lyophilised composition according to the first aspect to provide a human control in the method. The composition may comprise primers directed to a human control target nucleic acid. A human control target nucleic acid is a DNA sequence from human genomic DNA. This composition may be exposed to test sample and subjected to the same conditions as in steps c and c′. Nucleic acid amplification should occur if human DNA is present in the test sample and the color change associated with a positive result for the presence of target nucleic acid should therefore occur if the test sample contains human DNA. Accordingly, this can be used to determine the presence of human DNA in a test sample derived from a biological sample. Where a LAMP reaction is used to detect the presence or absence of a target nucleic acid (for example from a pathogen) in a sample taken from a human patient, the human control may be used to confirm (i) that an adequate sample was taken from the patient; and (ii) that the sample preparation process was performed correctly. A negative result (no color change) indicates the absence of human DNA and, therefore, that at least one of steps (i) or (ii) failed.

The method may therefore further comprise the steps of
a″. providing a lyophilised composition according to the first aspect in a third vessel, wherein the composition comprises nucleic acid amplification reagents directed to a human control target nucleic acid;
b″. rehydrating the lyophilised composition within the third vessel and adding the test sample to the third vessel to provide a rehydrated reaction mixture;
c″. subjecting the rehydrated reaction mixture to the same conditions as in step c; and
d″. determining a positive result for the presence of a human control target nucleic acid in the sample if a colorimetric change occurs due to the presence of nucleic acid amplification.

The method may further comprise confirming that the test sample is a valid sample (e.g. that an adequate sample was taken from a human patient; and that sample preparation was performed correctly) if a positive result is observed in step d. The method may further comprise comparing the color of the reaction mixture in the first, second and third vessels after steps c, c′ and c″ and i) confirming the presence of a target nucleic acid in the sample if a colorimetric change occurs in the first vessel but not in the second vessel or the absence of target nucleic acid in the sample if no colorimetric change occurs in the first vessel; and ii) confirming that the sample contains human DNA if a colorimetric change occurs in the third vessel. Confirmation step ii) may act to confirm that the sample is a valid sample from a human source.

The method may further involve use of a lyophilised composition according to the first aspect to provide an internal control in the method. The composition may comprise nucleic acid amplification reagents directed to an internal control template nucleic acid sequence. The internal control template nucleic acid sequence may be a nucleic sequence of non-human origin. The internal control template nucleic acid may be a synthetic nucleic acid, for example comprising a sequence based on a non-human animal gene. The internal control template nucleic acid sequence may be a synthetic non-biological sequence, i.e. fully artificial and not based on any natural gene. The lyophilised composition may include the internal control template nucleic acid from the outset (i.e. within the lyophilised composition) or the internal control template nucleic acid may be added to the test sample. The lyophilised composition may be exposed to test sample and subjected to the same conditions as in steps c, c′ and c″. Nucleic acid amplification should occur due to the presence of internal control template nucleic acid and the color change associated with a positive result for the presence of target nucleic acid should occur. Accordingly, this can be used as a positive control, for example to confirm that reagents have been stored correctly and not damaged at any stage prior to or while running the amplification reaction, that suitable reactions conditions (e.g. temperature and time period) have been used for amplification to occur, and, that the test sample is free from significant inhibitory contaminants.

The method may therefore further comprise the steps of
a′″. providing a lyophilised composition according to the first aspect in a fourth vessel, wherein the composition comprises nucleic acid amplification reagents directed to an internal control template nucleic acid;
b′″. rehydrating the lyophilised composition within the fourth vessel and adding test sample to the fourth vessel to provide a rehydrated reaction mixture;
c′″. subjecting the rehydrated reaction mixture to the same conditions as in step c; and
d′″. determining the presence of a positive result if a colorimetric change occurs due to the presence of nucleic acid amplification,
wherein an internal control template nucleic acid sequence is present within the lyophilised composition or is added in step b′″.

Where the internal control template nucleic acid is added in step b′″, it may be added to the test sample before the test sample is added.

The method may further comprise confirming that positive results determined any of steps d or d″ are valid is a positive result is observed in step d′″.

Any of steps a-a′″, b-b′″, c-c′″ and d-d′″ may be carried out concurrently.

Features of step b may apply to any of steps b′-b′″ mutatis mutandis. In any of steps b-b′″, rehydration of the lyophilised reaction mixture and addition of the test sample may be carried out as a single step, by addition of a liquid test sample, for example extracted RNA or DNA within a sample eluent (e.g. water, such as nuclease-free water), to the lyophilised reaction mixture. Rehydration may occur to any volume, for example up to about 1 mL, or about 1-100 μL, 1-50 μL, 10-30 μL or 20 μL.

The test sample used in steps b-b′″ may be the same test sample (e.g. aliquots dispensed from the same test sample).

In an eighth aspect, provided herein is a method for diagnosing infection by a pathogen in a subject, the method comprising:
a. providing a lyophilised composition according to the first aspect, wherein the lyophilised composition contains primers directed to a target nucleic acid of the pathogen;
b. rehydrating the lyophilised composition and adding a test sample obtained from the subject to provide a rehydrated reaction mixture;
c. subjecting the rehydrated reaction mixture to conditions suitable to allow nucleic acid amplification to occur if the target nucleic acid is present in the test sample; and
d. determining a positive result for the presence of the target nucleic acid in the test sample if a colorimetric change occurs due to the presence of nucleic acid amplification; and
e. diagnosing infection by the pathogen on the basis of a positive result.

In a ninth aspect, provided herein is a method for treating infection by a pathogen in a subject, the method comprising:
a. providing a lyophilised composition according to the first aspect, wherein the lyophilised composition contains primers directed to a target nucleic acid of the pathogen;
b. rehydrating the lyophilised composition and adding a test sample from a subject to provide a rehydrated reaction mixture;
c. subjecting the rehydrated reaction mixture to conditions suitable to allow nucleic acid amplification to occur if the target nucleic acid is present in the sample;
d. determining a positive result for the presence of the target nucleic acid in the sample if a colorimetric change occurs due to the presence of nucleic acid amplification; and
e. diagnosing infection by the pathogen in the subject on the basis of a positive result and treating the subject for said infection.

The subject may be a human subject. Treatment may comprise administering a therapeutic agent to the subject. The therapeutic agent may be a therapeutic agent suitable for treating infection by the pathogen and/or for treating one or more symptoms caused by infection by the pathogen. For example, if the pathogen is a virus, the therapeutic agent may be an anti-viral agent.

The test sample referenced in either of the eighth and ninth aspects may comprise extracted nucleic acids (RNA and/or DNA) from a biological from the patient or, alternatively, a biological sample from the patient may be used directly as the test sample, without extraction of nucleic acid. Preferably, the test sample comprises extracted nucleic acids (RNA and/or DNA) from a biological sample from the patient. The biological sample may be, for example, a nasopharyngeal swab, or a blood, a urine, a saliva, a tissue, stool, serum, lymph, semen, sweat, tears, amniotic fluid or wound exudate sample, or any other bodily fluid.

Any features of the compositions of the first, second and fifth aspects as described herein may apply mutatis mutandis to the methods of the seventh to ninth aspects. Any features of the method of the seventh aspect described herein may apply mutatis mutandis to the methods of the eighth and ninth aspects. Any features of steps a-d of the method of the seventh aspect described herein may apply mutatis mutandis to steps a-d of the method of the eighth and ninth aspects. The method of the eighth and ninth aspects may also comprise the further color control, human control and internal control steps as described for the seventh aspect.

In a tenth aspect, provided herein is a kit for carrying out a nucleic amplification reaction, the kit comprising a vessel having a lyophilised composition of the first aspect contained therein. The lyophilised composition may comprise nucleic acid amplification reagents that enable amplification of a target nucleic acid to occur, wherein the target nucleic acid is a nucleic acid sequence from a pathogen, such as a characteristic nucleic acid of a particular virus, for example, but not limited to, for example SARS-CoV-2, influenza, respiratory syncytial virus (RSV), human rhinovirus (HR), adenovirus or parainfluenza.

The kit may additionally comprise a vessel having a lyophilised composition for use as a color control composition in a colorimetric nucleic acid amplification reaction of the second aspect contained therein.

The kit may additionally comprise a vessel having a lyophilised composition of the first aspect contained therein, wherein the lyophilised composition comprises nucleic acid amplification reagents components that enable amplification of a target nucleic acid to occur, wherein the target nucleic acid is a human gene. This composition may act as a positive control, to validate sample identity and quality, e.g. successful extraction of a sample.

The kit may comprise more than one vessel having a lyophilised composition of the first aspect contained therein, wherein the lyophilised composition comprises nucleic acid amplification reagents that enable amplification of a target nucleic acid to occur, wherein the target nucleic acid is a nucleic acid sequence from a pathogen. The more than one vessel may each comprise nucleic acid reagents that enable amplification of a target nucleic acid from a different pathogen. This enables the kit to be used to detect for the presence of a panel of different pathogens.

In an eleventh aspect, provided herein is a pre-lyophilisation formulation comprising nucleic acid amplification reagents; a cryoprotectant; a pH adjusting agent; a colorimetric amplification indicator; and water. The pre-lyophilization formulation may be as described in relation to the first aspect. Any features of the pre-lyophilisation formulations as described in relation to the first aspect may apply mutatis mutandis to the eleventh aspects.

In any aspect described herein, the nucleic acid amplification reaction may preferably be an isothermal nucleic acid amplification method, preferably a Loop-Mediated Isothermal Amplification (LAMP) reaction.

As used herein, singular forms “a,” “an” and “the” also include plural forms unless the context clearly dictates otherwise. Use of the singular includes the plural unless specifically stated otherwise. The terms “comprising”, “containing” and “including” as well as other forms (e.g., “include,” “comprise” and “contain”) are not limiting. As used herein, “comprising” may also include “consisting essentially of” and “consisting of”.

As used herein, the term “about” is used in relation to numerical ranges and values. About also includes the exact value. Hence “about 20%” means “about 20%” and also “20%”. Generally, the term “about” can include an amount that would be expected to be within experimental error. When the term “about” is used at the start of a listing of ranges and/or values (such as “about 1-10% w/v, 1-5% w/v, or 2% w/v”), the term “about” is intended to apply to all ranges and values in that listing.

The terms “nucleic acid” and “nucleic acid molecule”, used interchangeably herein, refer to a molecule comprised of nucleotides, i.e., ribonucleotides, deoxyribonucleotides, or both. Nucleic acids include monomers and polymers of ribonucleotides, deoxyribonucleotides, or both, with the ribonucleotide and/or deoxyribonucleotides being connected together, in the case of the polymers, via 5′ to 3′ linkages. However, linkages may include any of the linkages known in the nucleic acid synthesis art including, for example, nucleic acids comprising 5′ to 2′ linkages. Nucleotides in a nucleic acid may be naturally occurring or synthetic analogues that are capable of forming base-pair relationships with naturally occurring base pairs. Examples of non-naturally occurring bases that are capable of forming base-pairing relationships include, but are not limited to, aza and deaza pyrimidine or purine analogues, and other heterocyclic base analogues, for example wherein one or more of the carbon and nitrogen atoms of the pyrimidine and purine rings have been substituted by a heteroatom, e.g., O, S, Se, P, etc.

A nucleic acid amplification reaction may be an isothermal nucleic acid amplification method. Isothermal amplification is a form of nucleic acid amplification which does not rely on the thermal denaturation of the target nucleic acid during the amplification reaction and hence may not require multiple rapid changes in temperature. This means that isothermal nucleic acid amplification methods can be carried out inside or outside of a laboratory environment. Various isothermal nucleic acid amplification methods have been developed, including but not limited to Loop-Mediated Isothermal Amplification (LAMP), for example tandem repeat LAMP, Strand Displacement Amplification (SDA), Transcription Mediated Amplification (TMA), Nucleic Acid Sequence Based Amplification (NASBA), Recombinase Polymerase Amplification (RPA), Rolling Circle Amplification (RCA), Ramification Amplification (RAM), Helicase-Dependent Isothermal DNA Amplification (HDA), Circular Helicase-Dependent Amplification (cHDA), Single Primer Isothermal Amplification (SPIA), Signal Mediated Amplification of RNA Technology (SMART), Self-Sustained Sequence Replication (3SR), Genome Exponential Amplification Reaction (GEAR) and Isothermal Multiple Displacement Amplification (IMDA). Further examples of such amplification technologies are described in, for example, (“Isothermal nucleic acid amplification technologies for point-of-care diagnostics: a critical review, Pascal Craw and Wamadeva Balachandrana Lab Chip, 2012, 12, 2469-2486, DOI:10.1039/C2LC40100B,”) incorporated here in its entirety by reference. Preferably, the amplification reaction is Loop-Mediated Isothermal Amplification (LAMP).

Alternatively, nucleic acid amplification reaction may be a polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, or thermal asymmetric interlaced PCR (TAIL-PCR).

The term “primer” as used herein refers to a nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e. in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, depending on the complexity of the target sequence, a nucleic acid primer typically contains at least 13 to 15 or more nucleotides, for example 13 to 60, 15 to 60 or 20 to 50 nucleotides, although it may contain fewer or more nucleotides.

Primers may be any primers suitable for acting as the point of initiation of amplification of a target nucleic acid. Primers may be any primers suitable acting as the point of initiation in a LAMP reaction. For example, primers may be FIP (forward inner primer), BIP (backward inner primer), F3 (forward outer primer), B3 (backward outer primer), LF (forward loop primer) and/or BF (backward loop primer) primers, for example, as described in be WO2019/234252, the contents of which are incorporated herein by reference in their entirety.

A “nucleotide triphosphate mixture” as referenced herein refers to a mixture of nucleotide triphosphates containing all nitrogenous bases necessary to enable nucleic acid amplification. The nucleotide triphosphate mixture may comprise deoxynucleotide triphosphates (dNTPs), nucleoside triphosphates (NTPs) or modified versions thereof. dNTPs comprise dATP, dGTP, dCTP, and dTTP.

A “colorimetric amplification indicator” as referenced herein refers to a compound that changes colorimetric properties in the presence of amplified nucleic acid or in the presence of by-products of nucleic acid amplification such as protons (H⁺) or metal ions. Such indicators may experience a change in their configuration as a result of a change in the concentration of protons (H⁺) or metal ions in a solution, resulting in a color change of the solution.

An amplification indicator can be a metal ion indicator (also called complexometric indicator or metallochromic indicator). Metal ion indicators may form a reversible complex with metal ions. A metal ion indicator is a substance that results in a color change in solution after forming a metal ion complex compared to color of the uncomplexed indicator. Examples of such metal ions include, but are not limited to, Ca²⁺, Mg²⁺, Zn²⁺ and other metal ions.

Suitable metal ion amplification indicators include, but are not limited to, hydroxynaphthol blue, eriochrome black t, calmagite, curcumin, fast sulphon black, hematoxylin, murexide, xylenon orange, BAPTA, BAPTA AM, BTC, BTC AM, Calcein, Calcein AM, Calcein Blue, Calcium Green 1, Calcium Green 2, Calcium Green 5N, Coelenterazine, Coelenterazine cp, Coelenterazine f, Coelenterazine h, Coelenterazine hep, Coelenterazine n, CoroNa Green, Corona Green AM, CoroNa Red, DAF FM, Fluo 3, Fluo 3 AM, PBFI AM, Phen Green SK, Quin 2, Quin 2 AM, RhodZin 3.

An amplification indicator can be a pH indicator. A pH indicator is a chemical detector for hydrogen ions (H+) or hydronium ions (H₃0+). pH indicators are organic molecules with an electronic configuration capable of transitioning in the visible spectra depending on the proton H+ concentration in solution. A pH indicator may be a weak acid or base whose configuration resides on the relationship between the concentration of the dissociated acid and its non-dissociated acid form. A pH indicator may cause the color of the solution to change depending on the pH. Suitable pH indicators include, but are not limited to: gentian violet, malachite green, thymol blue, methyl yellow, bromophenol blue, congo red, methyl orange, screened methyl orange (first transition), screened methyl orange (second transition), Bromocresol green, methyl red, methyl Purple, azolitmin red, bromocresol purple, bromothymol blue, phenol red, neutral red, naphtholphthalein, Cresol red, Cresolphthalein, Phenolphthalein, Thymolphthalein, Alizarine Yellow R yellow, Indigo carmine. The pH indicator may be a phenol red pH indicator. The phenol red pH indicator may allow visual detection of the accumulation of protons during the amplification reaction, providing a change in solution color from pink (negative) to yellow (positive).

An amplification indicator can be a redox indicator (also called an oxidation-reduction indicator), which is an indicator dye that undergoes a definite color change at a specific electrode potential. There are two common types of redox indicator: pH independent redox indicator and pH dependent redox.

Exemplary pH independent redox indicators include, but are not limited to, 2,2′-bipyridine, Nitrophenanthroline, N-Phenylanthranilic acid, 1, 10-Phenanthroline iron(II) sulfate complex, N-Ethoxychrysoidine, 2,2′-Bipyridine, 5,6-Dimethylphenanthroline, o-Dianisidine, Sodium diphenylamine sulfonate, Diphenylbenzidine, Diphenylamine, Viologen.

Examples of pH dependent redox indicators include, but are not limited to, Sodium 2,6-Dibromophenol-indophenol, Sodium o-Cresol indophenol, Thionine, Methylene blue, Indigotetrasulfonic acid, Indigotrisulfonic acid, Indigo carmine, Indigomono sulfonic acid, Phenosafranin, Safranin, Neutral red.

As used herein, “lyophilisation” refers to a dehydration process conducted at low temperature that involves freezing a sample (referred to herein as a pre-lyophilization formulation), lowering the pressure and the resultant removal of water by sublimation. Lyophilisation is also referred to as “freeze-drying”. The process generally involves three stages: “initial freezing” (also called “pre freezing”), where the sample is cooled to a freezing temperature (also referred to as a freeze temperature) at which the sample is frozen solid; “primary drying” which is a process of lowering pressure while the frozen sample is maintained at a freezing temperature and removing ice crystals by sublimation; and “secondary drying” which refers to a subsequent increase in temperature used to remove any remaining water molecules. The freezing temperature should preferably be below the collapse temperature (Tc) of the sample. This is beneficial to reduce the likelihood of collapse of the composition during drying. Maintaining a freezing temperature below, but close to the collapse temperature may be beneficial for efficiency of the drying cycle. The freezing temperature in pre-freezing and primary drying may be the same or different. For example, an increase in the freezing temperature may occur after pre-freezing, so that primary drying is carried out at a higher freezing temperature than the freezing temperature reaching in pre-freezing. Alternatively, primary drying may be carried out at the same freezing temperature as reached in pre-freezing.

As used herein, “ramp-freezing” refers to a lyophilisation process in which following loading of the sample, the temperature is gradually lowered (ramped) to the freezing temperature, before primary drying at a freezing temperature. For example, this may involve loading a sample to a freeze dryer and then reducing the temperature to the freezing temperature (e.g −50° C.) over a time period. The time period may be at least 1 hour, for example a time period of 1-4 hours, 2-3 hours or 2 hours.

As used herein, “flash-freezing” refers to a lyophilisation process in which the sample is subjected to a freezing temperature extremely quickly. For example, in the pre-freezing step, this may be achieved by loading sample to a freeze dryer already held at the freezing temperature. The sample may be held at that temperature for a time period before primary drying. The time period may be at least at least 1 hour, for example a time period of 1-4 hours, 2-4 hours or 3 hours.

As used herein, “annealing-freezing” refers to a lyophilisation process in which, following pre-freezing, the temperature of the sample is raised and then decreased again to a freezing temperature before the primary drying stage. An annealing stage may be included in either a ramp-freezing or a flash-freezing freeze-drying process.

A “vessel” as used herein should be interpreted broadly, to encompass any appropriate vessel that may be used for containing or processing a sample, a derivative of a sample, a composition and/or a reaction mixture. The vessel may be any suitable container, for example a sample tube or a well in a multi-well plate. If multiple vessels are present, each vessel may be an individual sample tube, for example in a strip of sample tubes, or each vessel may be an individual well in a multi-well plate. The vessel may be of any suitable volume. The vessel may be a sample tube. The term “sample tube” as used herein should be interpreted broadly, to encompass any appropriate tube that may be used for containing or processing a sample or a derivative of a sample. A vessel may be transparent or translucent to enable detection of a color change within the tube.

A “test sample” as used herein may be any suitable sample comprising a nucleic acid or suspected of comprising a nucleic acid. The test sample may be an aqueous liquid test sample comprising nucleic acid, or suspected of comprising nucleic acid, in an aqueous solvent or eluent, e.g. water. The terms eluent and solvent may be used interchangeably in relation to the test sample. For example, the sample may comprise extracted nucleic acids (RNA and/or DNA) from any type of biological (e.g. clinical) or environmental sample in a sample eluent. The sample may also be a sample of synthetic DNA (such as gBIocks) or a sample of a plasmid (e.g. containing a gene or gene fragment of interest). A sample may be obtained by any suitable method. It is noted that the sample may comprise a target nucleic acid and or other nucleic acids.

An environmental sample may be a sample from air, water, animal matter, plant matter or a surface. An environmental sample from water may be salt water, brackish water or fresh water. For example, an environmental sample from salt water may be from an ocean, sea or salt marsh. An environmental sample from brackish water may be from an estuary. An environmental sample from fresh water may be from a natural source such as a puddle, pond, stream, river, lake. An environmental sample from fresh water may also be from a man-made source such as a water supply system, a storage tank, a canal or a reservoir. An environmental sample from plant matter may, for example, be from a foodstock, a plant bulb or a plant seed. An environmental sample from a surface may be from an indoor or an outdoor surface. For example, the outdoor surface be soil or compost. The indoor surface may, for example, be from a hospital, such as an operating theatre or surgical equipment, or from a dwelling, such as a food preparation area, food preparation equipment or utensils. The environmental sample may contain or be suspected of containing a pathogen. Accordingly, the nucleic acid may be a nucleic acid from the pathogen.

A biological sample may be a clinical sample from a patient, e.g. a human patient. The sample may be a sample from a bodily fluid. The sample may be from blood, serum, lymph, urine, faeces, semen, sweat, tears, amniotic fluid, wound exudate or any other bodily fluid or secretion in a state of heath or disease. The sample may be a sample of cells or a cellular sample. The sample may comprise cells. The sample may be a tissue sample. The sample may be a biopsy. The sample from a patient may contain or be suspected of containing a pathogen. Accordingly, the target nucleic acid may be a nucleic acid from the pathogen.

A biological sample may be a sample from an animal, for example, from a bodily fluid of an animal (as described above) from a dead animal or a biopsy of a live animal.

Nucleic acids may be isolated, extracted and/or purified from a sample prior to use. The isolation, extraction and/or purification may be performed by any suitable technique. For example, nucleic acid isolation, extraction and/or purification may be performed using a nucleic acid isolation kit, a nucleic acid extraction kit or a nucleic acid purification kit, respectively.

Methods described herein may further comprise an initial step of isolating, extracting and/or purifying nucleic acids from a biological or environmental sample to provide a test sample. The method may therefore further comprise isolating nucleic acids from a biological or environmental sample. The method may further comprise extracting nucleic acids from the biological or environmental sample. The method may further comprise purifying nucleic acids from the biological or environmental sample. Alternatively, methods described herein may comprise direct amplification from a biological or environmental sample without an initial step of isolating, extracting and/or purifying nucleic acids from the biological or environmental sample. In such methods, a biological or environmental may be used as the test sample.

Following isolation, extraction and/or purification, a nucleic acid may be used immediately or may be stored under suitable conditions prior to use. Accordingly, the method may further comprise a step of storing an extracted nucleic acid and before use in an amplification reaction.

A “cryoprotectant” as used herein is an agent that acts as a protective agent in the lyophilisation process. Examples of cryoprotectants include polyhydroxy reagents (such as glycerin, mannitol, sorbitol, inositol, and polyethylene glycol) and sugar reagents (such as disaccharides, for example trehalose and sucrose or polysaccharides, for example dextran and ficoll).

A “polyhydroxy reagent” as used herein is a polyhydroxy compound, i.e. a compound containing two or more containing two or more hydroxyl groups per molecule. Examples of polyhydroxy reagents include sugar alcohols.

A “sugar reagent” is a reagent comprising a monosaccharide, disaccharide, oligosaccharide or polysaccharide-moiety. The reagent may be a monosaccharide, disaccharide, oligosaccharide or polysaccharide or it may comprise a monosaccharide, disaccharide, oligosaccharide or polysaccharide, for example as part of a copolymer formed from polymerisation of a monosaccharide, disaccharide, oligosaccharide or polysaccharide with another component.

A “sugar-based surfactant” is a surfactant comprising a monosaccharide, disaccharide, oligosaccharide or polysaccharide-moiety.

A “sugar alcohol” is an organic compound, typically derived from a sugar, containing one hydroxyl group attached to each carbon atom. A sugar alcohol may have the formula HOCH₂(CHOH)ₙCH₂OH. may, for example, be a sugar alcohol sugar alcohol-based surfactant. They may preferably comprise 2-12, 3-12, 4-12 or 5-12 carbon atoms, for example, 5, 6, 10, 11 or 12 carbon atoms.

“Ficoll” as used herein refers to a polymer of sucrose formed by copolymerisation of sucrose and epichlorohydrin. Ficoll 400 (CAS No. 26873-85-8), also known as Polysucrose 400, is a nonionic synthetic polymer of sucrose formed by copolymerisation of sucrose and epichlorohydrin. Ficoll 400 may have a molecular weight (Mr, relative molecular mass) of about 3×10⁵ to 5×10⁵ g/mol. Ficoll 400 may have a molecular weight of 400,000+/−100,000 as determined by intrinsic viscosity. Ficoll 400 may have an average molecular weight (e.g. weight average molecular weight (M_{w})) of ˜400000 g/mol (for example 380000-440000 g/mol). Molecular weight may be determined by size exclusion chromatography or light scattering techniques.

Dextran 70 is a dextran polysaccharide with an average molecular weight of approximately 75000 Daltons. The average molecular weight may be the weight average molecular weight (M_{w}) as determined by aqueous size exclusion chromatography, measured for example according to the United States Pharmacopeia (USP) or European Pharmacopoeia (EP).

Brij-35 (CAS No. 9002-92-0) is a polyoxyethylene lauryl ether of formula (C₂H₄O)ₙC₁₂H₂₆O.

Tergitol (CAS No 127087-87-0) is a nonylphenol polyethylene glycol ether of formula C₉H₁₉C₆H₄(OCH₂CH₂)₉OH.

The disclosure provides aspects and embodiments as set out in the following clauses:
1) A lyophilized composition, the composition comprising: nucleic acid amplification reagents; a cryoprotectant; a pH adjusting agent; and a colorimetric amplification indicator.
2) The composition of clause 1), wherein the nucleic acid amplification reagents comprise: a nucleic acid polymerase (e.g. a DNA polymerase); a nucleotide triphosphate mixture; at least two primers directed to a target nucleic acid; a source of a metal cofactor for the nucleic acid polymerase, and optionally a reverse transcriptase.
3) The composition of clauses 1) or 2), wherein the target nucleic acid is a nucleic acid sequence from a pathogen, for example, but not limited to, SARS-CoV-2, influenza, respiratory syncytial virus (RSV), human rhinovirus (HR), adenovirus or parainfluenza.
4) The composition of any preceding clause, wherein the target nucleic acid is a nucleic acid sequence from a human gene.
5) The composition of any one of clauses 2) to 4), wherein the source of a metal cofactor for the nucleic acid polymerase is a metal salt, for example a divalent metal salt, for example a salt comprising Mg²⁺, Ca²⁺ or Mn²⁺ ions.
6) The composition of clause 5), wherein the metal salt is a magnesium salt
7) The composition of clause 6), wherein the magnesium salt is magnesium sulphate.
8) The composition of any preceding clause, wherein the colorimetric amplification indicator is a pH sensitive indicator or a metal sensitive indicator, or a combination of one or more pH sensitive or metal sensitive indicators.
9) The composition of any preceding clause, wherein the colorimetric amplification indicator is (a) a pH indicator selected from gentian violet, malachite green, thymol blue, methyl yellow, bromophenol blue, congo red, methyl orange, methyl orange bromocresol green, methyl red, methyl Purple, azolitmin red, bromocresol purple, bromothymol blue, phenol red, neutral red, naphtholphthalein, Cresol red, Cresolphthalein, Phenolphthalein, Thymolphthalein, Alizarine Yellow R yellow or Indigo carmine; (b) a metal sensitive indicator selected from hydroxynaphthol blue, eriochrome black t, calmagite, curcumin, fast sulphon black, hematoxylin, murexide, xylenon orange, BAPTA, BAPTA AM, BTC, BTC AM, Calcein, Calcein AM, Calcein Blue, Calcium Green 1, Calcium Green 2, Calcium Green 5N, Coelenterazine, Coelenterazine cp, Coelenterazine f, Coelenterazine h, Coelenterazine hep, Coelenterazine n, CoroNa Green, Corona Green AM, CoroNa Red, DAF FM, Fluo 3, Fluo 3 AM, PBFI AM, Phen Green SK, Quin 2, Quin 2 AM, RhodZin 3, hydroxynaphthol blue or calmagite; or (c) a combination of any two or more of these indicators.
10) The composition of any preceding clause, wherein the indicator is phenol red, thymol blue, Eriochrome Black T, or a combination thereof.
11) The composition of any preceding clause, wherein the indicator is phenol red.
12) The composition of any preceding clause, comprising both a pH sensitive indicator and a metal sensitive indicator, for example thymol blue and Eriochrome Black T.
13) The composition of any preceding clause, wherein the solution formed by rehydration of the lyophilized composition is not pH buffered or has low pH buffering capacity.
14) The composition of any preceding clause, wherein the solution formed by rehydration of the lyophilized composition has a buffering capacity not more than that provided by a 7.5 mM solution of Tris (tris(hydroxymethyl)aminomethane) buffer with a starting pH of 8.8.
15) The composition of any preceding clause, wherein the composition is substantially free from Tris buffer.
16) The composition of any preceding clause, wherein the pH adjusting agent is a pH raising agent.
17) The composition of any preceding clause, wherein the pH adjusting agent comprises a base or any other compound that provides an overall pH raising effect when dissolved in water.
18) The composition of any preceding clause, wherein the pH adjusting agent is a metal hydroxide (e.g. NaOH or KOH), ammonia, calcium carbonate, sodium acetate, potassium cyanide or sodium sulfide, or a mixture thereof.
19) The composition of any preceding clause, wherein the pH adjusting agent is NaOH or KOH, preferably NaOH.
20) The composition of any preceding clause, wherein the cryoprotectant may be:
   a) a sugar reagent or a polyhydroxy reagent; and/or
   b) a polysaccharide or polysaccharide-containing moiety; and/or
   c) a surfactant, preferably a non-ionic surfactant.
21) The composition of any preceding clause, wherein cryoprotectant is a sugar-based or sugar alcohol-based surfactant.
22) The composition of any preceding clause, wherein the composition comprises one or more of trehalose, dextran, mannitol, a polymer of sucrose formed by copolymerisation of sucrose and epichlorohydrin (ficoll), sucrose, and sorbitol, preferably one or more of trehalose, dextran, mannitol, and a polymer of sucrose formed by copolymerisation of sucrose and epichlorohydrin (ficoll).
23) The composition of any preceding clause, wherein the composition comprises more than one cryoprotectant.
24) The composition of any preceding clause, wherein the composition comprises a combination of mannitol and ficoll (preferably D-mannitol and Ficoll 400) or a combination of dextran and trehalose (preferably dextran 70 and trehalose).
25) The composition of any preceding clause, wherein the pH adjusting agent is present in an amount sufficient to produce a rehydrated formulation with a target pH as recited herein, for example a target pH of about 7.5-9, preferably about 7.5-8.5, more preferably about 7.8 to 8.5, when the composition is rehydrated with water.
26) The composition of clause 25), wherein the composition comprises a source of a metal cofactor (e.g. MgSO₄) and a nucleic acid polymerase, and wherein the pH adjusting agent and the source of a metal cofactor (e.g. MgSO₄) are present in the composition at relative amounts sufficient such that, on rehydration with water, a formulation with a target pH, and a concentration of the source of a metal cofactor (e.g. MgSO₄) that is tolerable for activity of the nucleic acid polymerase, can be formed.
27) The composition of clause 25 or 26), wherein the composition comprises a source of a metal cofactor (e.g. MgSO₄) and a nucleic acid polymerase, and wherein the pH adjusting agent is present in an amount sufficient to produce a rehydrated formulation with a target pH, when rehydrated with a volume of water sufficient to provide a concentration of the source of a metal cofactor (e.g. MgSO₄) of about 4-20 mM, 4-15 mM, preferably about 4-10 mM, 4-8 mM, 6-8 mM, 4-7 mM or 6-7 mM in the rehydrated mixture.
28) The composition of any preceding clause, wherein the composition comprises a source of a metal cofactor (e.g. MgSO₄) and a nucleic acid polymerase, and wherein the composition is capable of being rehydrated to provide a rehydrated formulation comprising:
   a. the source of a metal cofactor (e.g. MgSO₄) at a concentration of about 4-20 mM, 4-15 mM, preferably about 4-10 mM, 4-8 mM, 6-8 mM, 4-7 mM or 6-7 mM; and
   b. the pH adjusting agent (e.g. NaOH) at a concentration sufficient to provide a target pH as recited herein, for example a pH of about 7.5-9, preferably about 7.5-8.5, or more preferably about 7.8 to 8.5.
29) The composition of any preceding clause, wherein the composition is capable of being rehydrated to provide a rehydrated formulation comprising:
   a. the source of a metal cofactor (e.g. MgSO₄) at a concentration of about 4-20 mM, 4-15 mM, preferably about 4-10 mM, 4-8 mM, 6-8 mM, 4-7 mM or 6-7 mM; and
   b. the pH adjusting agent (e.g. NaOH) at a concentration of at least about 5 mM, at least about 6 mM, at least about 7 mM, at least about 8 mM, at least about 8.5 mM, or at least about 8.75 mM.
30) The composition of any preceding clause, wherein pH adjusting agent in the rehydrated formulation is at a concentration of no more than about 20 mM, no more than about 15 mM, preferably no more than about 14 mM.
31) The composition of any preceding clause, wherein the composition is capable of being rehydrated to provide a rehydrated formulation comprising a cryoprotectant concentration of at least about 0.5% w/v, at least about 1% w/v or at least about 1.5% w/v.
32) The composition of any preceding clause, wherein the composition is capable of being rehydrated to provide a rehydrated formulation comprising a cryoprotectant concentration of up to about 20% w/v or up to about 15% w/v.
33) The composition of any preceding clause, wherein the composition is capable of being rehydrated to provide a rehydrated formulation comprising a cryoprotectant concentration of about 1-10% w/v.
34) The composition of any preceding clause, wherein the composition is capable of being rehydrated to provide a rehydrated formulation comprising a cryoprotectant concentration of about 1-5% w/v.
35) The composition of any preceding clause, wherein the composition is capable of being rehydrated to provide a rehydrated formulation comprising at least one of: a) a mannitol concentration of about 1-10% w/v, 1-5% w/v, 1-4% w/v, 1-3% w/v or 2% w/v, b) a ficoll concentration of about 0.5-5% w/v, 0.5-3% w/v, 0.5-2% w/v, 0.5-1.5% w/v or 1% w/v; c) a trehalose concentration of about 1-10% w/v, 1-5% w/v, 1-4% w/v, 1-3% w/v or 2% w/v; and d) a dextran concentration of about 0.5-10% w/v, 0.5-5% w/v, 0.5-4% w/v, 1-3% w/v or 2% w/v.
36) The composition of any preceding clause, wherein the composition is capable of being rehydrated to provide a rehydrated formulation comprising at least one of: a) a mannitol concentration of about 1-3% w/v, b) a ficoll concentration of about 0.5-1.5% w/v; c) a trehalose concentration of about 1-3% w/v; and d) a dextran concentration of about 1-3% w/v.
37) The composition of any of clauses 31) to 36), wherein the composition is capable of being rehydrated to provide a rehydrated formulation comprising the pH adjusting agent (e.g. NaOH) at a concentration of at least about 5 mM, at least about 6 mM, at least about 7 mM, at least about 8 mM, at least about 8.5 mM, or at least about 8.75 mM.
38) The composition of any preceding clause, wherein the composition comprises mannitol and ficoll, or dextran and trehalose.
39) The composition of any preceding clause, wherein the composition is capable of being rehydrated to provide a rehydrated formulation comprising the colorimetric amplification indicator at a concentration of at least about 50 μM.
40) The composition of any preceding clause, wherein the composition is capable of being rehydrated to provide a rehydrated formulation comprising the colorimetric amplification indicator at a concentration of 50-300 μM.
41) The composition of any preceding clause, wherein the composition is capable of being rehydrated to provide a rehydrated formulation comprising: a) phenol red at a concentration of about 50-100 μM; or b) thymol blue at a concentration of about 50-175 μM and EBT at a concentration of about 50-100 μM.
42) The composition of any preceding clause, wherein the composition is capable of being rehydrated to provide a rehydrated formulation comprising:
   a. the nucleic acid polymerase at a concentration of at least about 0.3 U/μL, 0.3-2 U/μL, 0.3-1 U/μL or 0.3-0.7 U/μL; and/or
   b. the reverse transcriptase enzyme, if present, at a concentration of at least about 0.15 U/μL, a concentration of about 0.15-5 U/μL, or a concentration of about 0.15-3.25 U/μL; and/or
   c. the nucleotide triphosphate mixture at a concentration of at least about 1 mM, or about 1 to 2 mM or 1.2 to 1.7 mM.
43) The composition of any preceding clause, wherein the composition is capable of being rehydrated to provide a rehydrated formulation comprising:
   a. the nucleic acid polymerase at a concentration of 0.3-0.7 U/μL; and/or
   b. the reverse transcriptase enzyme, if present, at a concentration of about 0.15-3.25 U/μL; and/or
   c. the nucleotide triphosphate mixture at a concentration of about 1.2 to 1.7 mM.
44) The composition of any preceding clause, wherein the composition further comprises an enzyme stabiliser, for example bovine serum albumin (BSA), DMSO, gelatin, Prionex (a polypeptide fraction of highly purified dermal collagen of porcine origin), sodium alginate, or a combination thereof.
45) The composition of clause 44), wherein the enzyme stabiliser is BSA.
46) The composition ofany preceding clause, wherein the composition is capable of being rehydrated to provide a rehydrated formulation comprising an enzyme stabilizer (e.g. BSA) at a concentration of about 0-10 mg/mL, 0-5 mg/mL, 0.5-10 mg/mL, 0.5-5 mg/mL, 1-10 mg/mL, 1-5 mg/mL, 1-3 mg/mL or 1-2 mg/mL.
47) The composition of clause 46), wherein the composition is capable of being rehydrated to provide a rehydrated formulation comprising an enzyme stabilizer (e.g. BSA) at a concentration of about 0-2 mg/mL or 1-2 mg/mL.
48) The composition of any preceding clause, wherein the composition further comprises at least one additive selected from Triton X-100, KC, (N₄)₂SO₄, Tween 20 (polyoxyethylene (20) sorbitan monolaurate), sodium azide, spermidine, tergitol, Brij-35, DMSO, formamide, PEG 35K, PEG 8K, GuHC, GuTC, dithiothreitol (DTT) and ethylenediaminetetraaceti acid (EDTA).
49) The composition of any preceding clause, wherein the composition is capable of being rehydrated to provide a rehydrated formulation comprising one or more of:

| Component | Concentration |
|---|---|
| Triton X-100 | about 0-5%, 0-3%, or 0-2% |
| Tween 20 | about 0-2%, or 0.1-1.8% |
| Tergitol | about 0-5%, 0-3%, 0-2%, or 0-1.5% |
| Brij-35 | about 0-10%, 0-5%, or 0-4% |
| DMSO | about 0-4% |
| Formamide | about 0-5%, 0-4%, or 0-2% |
| Sodium Azide | about 0-0.04% |
| Spermidine | about 0-0.1% |
| Polyethyleneglycol 35K | about 0-7% |
| Polyethyleneglycol 8K | about 0-10% |
| GuHCl | about 0-40 mM |
| GuTC | about 0-20 mM |
| Glycerol | about 0-2%, 0-1%, 0-0.5% |
| KCl | about 0-110 mM, 0-100 mM, 0-70 mM, |
| | 30-70 mM, or 40-60 mM |
| (NH₄)₂SO₄ | about 0-20 mM or 5-15 mM |

wherein all percentages are % w/v.
50) The composition of any preceding clause, wherein the composition is capable of being rehydrated to provide a rehydrated formulation comprising one or more of:

| Component | Concentration |
|---|---|
| Triton X-100 | about 0-2% |
| Tween 20 | about 0-1.8% |
| Tergitol | about 0-1.5% |
| Brij-35 | about 0-4% |
| DMSO | about 0-4% |
| Formamide | about 0-2% |
| Sodium Azide | about 0-0.04% |
| Spermidine | about 0-0.1% |
| Polyethyleneglycol 35K | about 0-7% |
| Polyethyleneglycol 8K | about 0-10% |
| GuHCl | about 0-40 mM |
| GuTC | about 0-20 mM |
| Glycerol | about 0-0.2% |
| KCl | about 40-60 mM |
| (NH₄)₂SO₄ | about 5-15 mM |

wherein all percentages are % w/v.
51) The composition of any preceding clause, wherein the composition is capable of being rehydrated to provide a rehydrated formulation with a low-buffering capacity, for example with a buffering capacity of no more than that provided by a 7.5 mM solution of Tris buffer with a starting pH of 8.8.
52) The composition of any preceding clause, wherein the composition is capable of being rehydrated to provide a rehydrated formulation with a buffering capacity of no more than that provided by a 5 mM solution of Tris buffer with a starting pH of 8.8.
53) The composition of any preceding clause, wherein the composition is capable of being rehydrated to provide a rehydrated formulation with a buffering capacity of no more than that provided by a 2.5 mM solution of Tris buffer with a starting pH of 8.8.
54) The composition of any preceding clause, wherein the composition is capable of being rehydrated to provide a rehydrated formulation with a buffering capacity of no more than that provided by a 1 mM solution of Tris buffer with a starting pH of 8.8.
55) The composition of any preceding clause, wherein the composition is capable of being rehydrated to provide a rehydrated formulation with a buffering capacity of no more than that provided by a 0.75 mM solution of Tris buffer with a starting pH of 8.8.
56) The composition of any preceding clause, wherein the composition is capable of being rehydrated to provide a rehydrated formulation with a buffering capacity of no more than that provided by a 0.5 mM solution of Tris buffer with a starting pH of 8.8.
57) The composition of any of clauses 51) to 56), wherein the buffering capacities may be measured when the composition is rehydrated with water sufficient to provide the source of a metal cofactor (e.g. MgSO₄) at a concentration of about 4-10 mM, 4-8 mM, 6-8 mM, 4-7 mM or 6-7 mM.
58) The composition of any preceding clause, wherein the composition is capable of being rehydrated to provide a rehydrated formulation comprising Tris buffer (e.g. TrisHCl) at a concentration of no more than about 7.5 mM, no more than about 2.5 mM, no more than about 1 mM, no more than about 0.75 mM or no more than about 0.5 mM, or a formulation absent Tris buffer.
59) The composition of any preceding clause, wherein the composition is capable of being rehydrated to provide a rehydrated formulation comprising Tris buffer (e.g. TrisHCl) at a concentration of no more than about 0.5 mM.
60) The composition of any preceding clause, wherein the composition is capable of being rehydrated to provide a rehydrated formulation with a glycerol concentration of no more than about 2% w/v, no more than about 1% w/v or no more than about 0.5% w/v.
61) The composition of any preceding clause, wherein the composition is capable of being rehydrated to provide a rehydrated formulation with a glycerol concentration of no more than about 2% w/v, no more than about 1% w/v or no more than about 0.5% w/v, when the composition is rehydrated with water sufficient to provide the source of a metal cofactor (e.g. MgSO₄) at a concentration of about 4-10 mM, 4-8 mM, 6-8 mM, 4-7 mM or 6-7 mM.
62) The composition of any preceding clause, wherein the composition is capable of being rehydrated to provide a rehydrated formulation with a glycerol concentration of no more than about 1% w/v.
63) The composition of any preceding clause, wherein the composition is substantially free of glycerol.
64) The composition of any preceding clause, wherein the composition further comprises an internal control template nucleic acid.
65) The composition of any preceding clause, wherein the internal control template nucleic acid is a nucleic acid of non-human origin, for example a synthetic nucleic acid, for example based on a non-human animal gene.
66) The composition of clause 64) or 65), wherein the internal control template nucleic acid is the target nucleic acid, the composition comprises primers directed to the internal control template nucleic acid sequence and, preferably, the composition does not comprise primers directed any other target nucleic acid.
67) The composition of any preceding clause, wherein the lyophilised composition is a composition obtainable by lyophilisation of a pre-lyophilisation formulation comprising the nucleic acid amplification reagents; cryoprotectant; pH adjusting agent; and colorimetric amplification indicator, and water.
68) The composition of clause 67), wherein the water comprises nuclease-free water.
69) The composition of clause 67) or 68), wherein the pre-lyophilisation formulation comprises a source of a metal cofactor (e.g. MgSO₄), and wherein the pre-lyophilisation formulation comprises the pH adjusting agent (e.g. NaOH) and the source of a metal cofactor (e.g. MgSO₄) present at a molar ratio of at least 1:1 (pH adjusting agent:cofactor).
70) The composition of clause 69), wherein the pH adjusting agent is present in the pre-lyophilisation formulation in a molar excess compared to the source of metal cofactor, for example wherein the pH adjusting agent:cofactor molar ratio is at least 1.05:1.
71) The composition of any of clauses 67)-70), wherein the pre-lyophilisation formulation comprises a source of a metal cofactor (e.g. MgSO₄), and wherein the pre-lyophilisation formulation comprises the source of a metal cofactor (e.g. MgSO₄) at a concentration of about 8-40 mM, 8-30 mM, 8-20 mM, 4-16 mM, 12-20 mM, 8-14 mM or 12-14 mM.
72) The composition of clause 71), wherein the pre-lyophilisation formulation comprises the source of a metal cofactor (e.g. MgSO₄) at a concentration of about 8-20 mM, 4-16 mM, 12-20 mM, 8-14 mM or 12-14 mM.
73) The composition of any of clauses 67)-72), wherein the pre-lyophilisation formulation comprises the pH adjusting agent (e.g. NaOH) at a concentration of at least about 10 mM, at least about 12 mM, at least about 14 mM, at least about 16 mM, at least about 17 mM, at least about 18 mM, or at least about 20 mM, for example wherein the pH adjusting agent is at a concentration of no more than about 40 mM, no more than about 30 mM, preferably no more than about 27 mM.
74) The composition of clause 73), wherein the pre-lyophilisation formulation comprises the pH adjusting agent (e.g. NaOH) at a concentration of about 17.5-35 mM.
75) The composition of clause 73), wherein the pre-lyophilisation formulation comprises the pH adjusting agent (e.g. NaOH) at a concentration of about 17.5-30 mM.
76) The composition of clause 73), wherein the pre-lyophilisation formulation comprises the pH adjusting agent (e.g. NaOH) at a concentration of about 17.5-28 mM.
77) The composition of clause 73), wherein the pre-lyophilisation formulation comprises the pH adjusting agent (e.g. NaOH) at a concentration of about, 19-35 mM or 20-35 mM.
78) The composition of clause 73), wherein the pre-lyophilisation formulation comprises the pH adjusting agent (e.g. NaOH) at a concentration of about, 19-30 mM or 20-30 mM.
79) The composition of clause 73), wherein the pre-lyophilisation formulation comprises the pH adjusting agent (e.g. NaOH) at a concentration of about 17.5-27 mM, 19-27 mM or 20-27 mM.
80) The composition of any of clauses 67)-79), wherein the pre-lyophilisation formulation comprises a cryoprotectant concentration of at least about 1% w/v, at least about 2% w/v or at least about 3% w/v, for example wherein the cryoprotectant concentration is up to about 40% w/v, up to about 30% w/v or up to about 20% w/v.
81) The composition of clause 80), wherein the pre-lyophilisation formulation comprises a cryoprotectant concentration of about 2-20% w/v.
82) The composition of clause 81), wherein the pre-lyophilisation formulation comprises a cryoprotectant concentration of about 2-10% w/v.
83) The composition of any of clauses 67)-82), wherein the pre-lyophilisation formulation comprises at least one of: a) a mannitol concentration of about 2-20% w/v, 2-10% w/v, 2-8% w/v, 2-6% w/v or 4% w/v; b) a Ficoll concentration of about 1-10% w/v, 1-6% w/v, 1-4% w/v, 1-3% w/v or 2% w/v; c) a trehalose concentration of about 2-20% w/v, 2-10% w/v, 2-8% w/v, 2-6% w/v or 4% w/v; and d) a dextran concentration of about 1-20% w/v, 1-10% w/v, 1-8% w/v, 2-6% w/v or about 4% w/v.
84) The composition of clause 83), wherein the pre-lyophilisation formulation comprises at least one of: a) a mannitol concentration of about 2-6% w/v, b) a ficoll concentration of about 1-3% w/v; c) a trehalose concentration of about 2-6% w/v; and d) a dextran concentration of about 2-6% w/v.
85) The composition of any of clauses 67)-84), wherein the pre-lyophilisation formulation comprises mannitol and ficoll, or dextran and trehalose.
86) The composition of any of clauses 67)-85), wherein the pre-lyophilisation formulation comprises the colorimetric amplification indicator at a concentration of at least about 100 μM.
87) The composition of any of clauses 67)-86), wherein the pre-lyophilisation formulation comprises the colorimetric amplification indicator at a concentration of about 100-600 μM.
88) The composition of any of clauses 67)-87), wherein the pre-lyophilisation formulation comprises: a) phenol red at a concentration of about 100-200 μM; or b) thymol blue at a concentration of about 100-350 μM and EBT at a concentration of about 100-200 μM.
89) The composition of any of clauses 67)-88), wherein the pre-lyophilisation formulation comprises:
   a. the nucleic acid polymerase at a concentration of at least about 0.6 U/μL, 0.6-4 U/μL, 0.6-2 U/μL, 0.6-1.4 U/μL or 1 U/μL; and/or
   b. the reverse transcriptase enzyme, if present, at a concentration of at least about 0.3 U/μL, a concentration of about 0.3-10 U/μL, a concentration of about 0.6-6.5 U/μL, or a concentration of about 0.9 U/μL; and/or
   c. the nucleotide triphosphate mixture at a concentration of at least about 2 mM, or about 2 to 4 mM, 2.4 to 3.4 mM or 2.8 mM.
90) The composition of any of clauses 67)-89), wherein the pre-lyophilisation formulation comprises:
   a. the nucleic acid polymerase at a concentration of about 0.6-1.4 U/μL; and/or
   b. the reverse transcriptase enzyme, if present, at a concentration about 0.3-6.5 U/μL; and/or
   c. the nucleotide triphosphate mixture at a concentration of about 2.4 to 3.4 mM.
91) The composition of any of clauses 67)-90), wherein the pre-lyophilisation formulation further comprises an enzyme stabiliser, for example wherein the enzyme stabiliser is bovine serum albumin (BSA), DMSO, gelatin, Prionex (a polypeptide fraction of highly purified dermal collagen of porcine origin), sodium alginate, or a combination thereof, preferably BSA.
92) The composition of clause 91), wherein the pre-lyophilisation formulation comprises an enzyme stabilizer (e.g. BSA) at a concentration of about 0-20 mg/mL, 0-10 mg/mL, 1-20 mg/mL, 1-10 mg/mL, 2-20 mg/mL, 2-10 mg/mL, 2-6 mg/mL, 2-4 mg/mL or 2.4 mg/mL.
93) The composition of clause 92), wherein the pre-lyophilisation formulation comprises an enzyme stabilizer (e.g. BSA) at a concentration of about 0-4 mg/mL or 2-4 mg/mL.
94) The composition of any of clauses 67)-93), wherein the pre-lyophilisation formulation further comprises at least one additive, selected from Triton X-100, KCl, (NH₄)₂SO₄, Tween 20 (polyoxyethylene (20) sorbitan monolaurate), sodium azide, spermidine, tergitol, Brij-35, DMSO, formamide, PEG 35K, PEG 8K, GuHCl, GuTC, dithiothreitol (DTT) and ethylenediaminetetraacetic acid (EDTA).
95) The composition of any of clauses 67)-94), wherein the pre-lyophilisation formulation comprises one or more of:

| Component | Concentration |
|---|---|
| Triton X-100 | about 0-10%, 0-6%, or 0-4% |
| Tween 20 | about 0-4%, or 0.2-3.6% |
| Tergitol | about 0-10%, 0-6%, 0-4%, or 0-3% |
| Brij-35 | about 0-20%, 0-10%, or 0-8% |
| DMSO | about 0-8% |
| Formamide | about 0-10%, 0-8%, or 0-4% |
| Sodium Azide | about 0-0.08% |
| Spermidine | about 0-0.2% |
| Polyethyleneglycol | about 0-14% |
| 35K | |
| Polyethyleneglycol | about 0-20% |
| 8K | |
| GuHCl | about 0-80 mM |
| GuTC | about 0-40 mM |
| Glycerol | about 0-4%, 0-2%, 0-1% |
| KCl | about 0-220 mM, 0-200 mM, 0-140 mM, |
| | 60-140 mM, or 80-120 mM |
| (NH₄)₂SO₄ | about 0-40 mM or 10-30 mM |

wherein all percentages are % w/v.
96) The composition of any of clauses 67)-95), wherein the pre-lyophilisation formulation comprises one or more of:

| Component | Concentration |
|---|---|
| Triton X-100 | about 0-4% |
| Tween 20 | about 0-3.6% |
| Tergitol | about 0-3% |
| Brij-35 | about 0-8% |
| DMSO | about 0-8% |
| Formamide | about 0-4% |
| Sodium Azide | about 0-0.08% |
| Spermidine | about 0-0.2% |
| Polyethyleneglycol | about 0-14% |
| 35K | |
| Polyethyleneglycol | about 0-20% |
| 8K | |
| GuHCl | about 0-80 mM |
| GuTC | about 0-40 mM |
| KCl | about 80-120 mM |
| (NH₄)₂SO₄ | about 10-30 mM |

wherein all percentages are % w/v.
97) The composition of any of clauses 67)-96), wherein the pre-lyophilisation formulation has a glycerol concentration of no more than about 4% w/v, no more than about 2% w/v or no more than about 1% w/v.
98) The composition of any of clauses 67)-97), wherein the pre-lyophilisation formulation is substantially free of glycerol.
99) The composition of any of clauses 67)-98), wherein the pre-lyophilisation formulation comprises Tris buffer (e.g. TrisHCl) at a concentration of 0-15 mM, 0-5 mM, 0-2 mM, 0-1.5 mM or 0-1 mM.
100) The composition of any of clauses 67)-99), wherein the pre-lyophilisation formulation comprises Tris buffer (e.g. TrisHCl) at a concentration of 0-1 mM.
101) The composition of any of clauses 67)-100), wherein the pre-lyophilisation formulation is provided in a diluted form, wherein the pre-lyophilisation formulation comprises the same components as any of clauses 67)-100), at the same relative concentrations, but with the concentrations of all components reduced by the same factor, for example a factor of 2.
102) The composition of any of clauses 67)-100), wherein the pre-lyophilisation formulation is provided in a concentrated form, wherein the pre-lyophilisation formulation comprises the same components as any of clauses 67)-100), at the same relative concentrations, but with the concentrations of all components increased by the same factor.
103) The composition of any of clauses 67)-102), wherein the pre-lyophilisation formulation has a pH of at least about 8.5, for example about 8.5-12.
104) The composition of any of clauses 67)-103), wherein the pre-lyophilisation formulation has a pH of about 8.5-11, or about 8.5-10.5.
105) The composition of any of clauses 67)-104), wherein the pre-lyophilisation formulation has a pH of at least about 9 or at least about 9.5, for example about 9.5-10.5.
106) A lyophilised composition for use as a color control composition in a colorimetric nucleic acid amplification reaction, wherein the composition corresponds to a composition of any of clauses 1)-105), but with the exclusion of at least one nucleic acid amplification reagent.
107) The composition of clause 106), wherein the composition is absent the presence of nucleic acid polymerase and/or reverse transcriptase.
108) A process for producing a lyophilized composition for use in a nucleic acid amplification reaction, the process comprising: providing a pre-lyophilisation formulation comprising nucleic acid amplification reagents, a cryoprotectant, a pH adjusting agent; a colorimetric amplification indicator, and water; and lyophilizing the formulation to produce a lyophilized composition.
109) The process of clause 108), wherein the pre-lyophilisation formulation is as defined in any of clauses 67) to 105).
110) The process of clause 108) or 109), wherein lyophilization is performed using a flash freeze-drying process or a ramp freeze-drying process, optionally wherein the freeze-drying process comprises an annealing stage.
111) The process of clause 110), wherein the freeze-drying process is a flash freeze-drying process, optionally comprising an annealing stage.
112) The process of any of clauses 108) to 111), wherein the freeze-drying process comprises initial freezing of the pre-lyophilisation formulation to a freeze temperature.
113) The process of clause 112), wherein:
   a) the freeze temperature is about −35° C. or lower, about −40° C. or lower, about −45° C. or lower or about −50° C. or lower, preferably about −40° C. or lower, about −45° C. or lower or about −50° C. or lower; and/or
   b) the freeze temperature is no lower than about −80° C., no lower than about −70° C. or no lower than about −60° C.; and/or
   c) the freeze temperature is below the collapse temperature (Tc) of the formulation, optionally within about 15° C., 10° C. or 5° C. of the collapse temperature; and/or
   d) the initial freezing is carried out as flash-freeze or a ramp-freeze, preferably as a flash freeze, where the pre-lyophilisation formulation is loaded to a freeze-dryer already cooled to the freeze temperature.
114) The process of any of clauses 112) to 113), wherein the temperature is ramped to the freeze temperature, optionally over a time period of at least about 1 hour, for example a time period of about 1-4 hours, 2-3 hours or 2 hours.
115) The process of any of clauses 112) to 114), wherein in the initial freezing step, the pre-lyophilisation formulation is loaded to a freeze dryer already at the freezing temperature and held at that temperature for a time period, optionally wherein the time period is at least about 1 hour, for example a time period of about 1-4 hours, 2-4 hours or 3 hours.
116) The process of any of clauses 112) to 114), wherein in the initial freezing step, the pre-lyophilisation formulation is loaded to a freeze dryer already at the freezing temperature and held at that temperature for a time period, optionally wherein the time period is at least about 1 hour, for example a time period of about 1-4 hours, 1-3 hours or 2 hours.
117) The process of any of clauses 112) to 116), wherein an annealing stage is present after initial freezing comprising increasing the temperature to an increased temperature of about −10° C. to −20° C., preferably about −15° C., and decreasing the temperature back to a freeze temperature.
118) The process of clause 117), wherein:
   a) the temperature is increased to the increased temperature over a time period of about 0.5-2 or 1-1.5 hours; and/or
   b) the temperature is held at the increased temperature for a time period of about 1-3 or 2 hours; and/or
   c) the temperate decreased to the freeze temperature over a period of about 0.5-2 or 1-1.5 hours; and/or
   d) after temperature decrease, the temperature is held at the freeze temperature for a time period of about 0.5-2 or 1 hours.
119) The process of any of clauses 112) to 118), wherein initial freezing and/or annealing is carried out at atmospheric pressure.
120) The process of any of clauses 112) to 119), comprising carrying out primary drying after initial freezing and after annealing, if present, at a freeze temperature and at a reduced pressure of no more than about 3000 μBar, no more than about 1000 μBar, no more than about 100 μBar, for example about 10-80 μBar, or about 20-60 μBar.
121) The process of clause 120), wherein the freeze temperature is:
   a) below the collapse temperature (Tc) of the formulation; and/or
   b) about −40° C. or lower, about −45° C. or lower or about −50° C. or lower; and/or
   c) no lower than about −70° C. or no lower than −60° C.; and/or
   d) the same as the freeze temperature of the initial freezing, or within about 10° C. or 5° C. of the freeze temperature of the initial freezing.
122) The process of any of clauses 120) to 121), wherein primary drying is for a time period before secondary drying of at least about 10 hours, at least about 20 hours, at least about 30 hours or at least 40 hours, for example a time period of about 20-100 hours, 40-80 hours or 40-60 hours.
123) The process of any of clauses 112) to 119), comprising carrying out primary drying after initial freezing and after annealing, if present, at a freeze temperature and at a reduced pressure of no more than about 3000 μBar, no more than about 1000 μBar, no more than about 200 μBar, for example about 10-200 μBar, or about 150-200 μBar (e.g. about 180 μBar).
124) The process of clause 123), wherein the freeze temperature is:
   a) below the collapse temperature (Tc) of the formulation; and/or
   b) about −25° C. or lower or about −30° C. or lower; and/or
   c) no lower than about −70° C. or no lower than −60° C.; and/or
   d) the same as the freeze temperature of the initial freezing, or within about 15° C., 10° C. or 5° C. of the freeze temperature of the initial freezing.
125) The process of clause 123) or 124), wherein primary drying is carried out at a first freeze temperature, followed by lowering to a second freeze temperature.
126) The process of clause 125), wherein primary drying comprises initial hold at a first freeze temperature that is the same as the freeze temperature of the initial freezing, or within about 10° C. or 5° C. of the freeze temperature of the initial freezing, followed by ramping to and holding at a second freeze temperature 5-10° C. lower than the first freeze temperature, optionally wherein the first freeze temperature is −35 to −45° C. and the second freeze temperature is −25 to −35° C.
127) The process of any of clauses 123) to 126), wherein primary drying is carried out for a time period before secondary drying of at least about 6 hours, at least about 10 hours, for example a time period of about 10-15 hours.
128) The process of any of clauses 120) to 127), comprising carrying out secondary drying a temperature of about 0° C. or higher, for example about 0-30° C., 10-30° C., 15-25° C. or about 20° C., optionally wherein secondary drying comprises a ramp step, where the temperature is increased from the primary drying temperature to the secondary drying temperature, followed by a hold step, held at the secondary temperature.
129) The process of clause 128), wherein:
   a) the ramp step is for a time period of least about 0.5 hours, at least about 1 hour, for example a time period of about 0.5-4 hours, 1-4 hours or 1-2 hours and/or the hold step is for a time period of at least about 2 hours, at least about 4 hours, for example a time period of 2-10 hours, 4-8 hours, 5-7 hours or about 6 hours; and/or
   b) secondary drying is carried out a pressure of no more than about 3000 μBar, no more than about 1000 μBar, no more than about 100 μBar, for example about 10-80 μBar, or 20-60. μBar, optionally wherein the pressure is the same as the pressure used in primary drying.
130) The process of clause 128), wherein:
   a) the ramp step is for a time period of least about 0.5 hours, at least about 1 hour, for example a time period of about 0.5-4 hours, 1-4 hours or 1-2 hours and/or the hold step is for a time period of at least about 2 hours, at least about 4 hours, for example a time period of 2-10 hours, 4-8 hours, 5-7 hours or about 6 hours; and/or
   b) secondary drying is carried out at a pressure of no more than about 3000 μBar, no more than about 1000 μBar, no more than about 200 μBar, for example about 10-200 μBar, or about 150-200 μBar (e.g. about 180 μBar), optionally wherein the pressure is the same as the pressure used in primary drying).
131) The process of any of clauses 128) to 130), wherein, after secondary drying, a step of aeration with an inert gas, e.g. N₂ gas, is carried out, optionally at a temperature of 0° C. or higher, for example about 0-30° C., 10-30° C., 15-25° C. or 20° C., optionally at the same temperature as secondary drying, optionally at atmospheric pressure.
132) The process of any of clauses 108) to 131), wherein the pre-lyophilisation formulation is provided in a vessel, e.g. a sample tube, such that lyophilisation provides a lyophilised composition contained within the vessel.
133) The process of any of clauses 108) to 132), wherein after lyophilization the lyophilised composition is transferred into an inert gas environment for packaging.
134) The process of clause 133), wherein packaging comprises closing the vessel in which the lyophilized composition has been formed, or placing the lyophilized composition in a vessel and closing it, under an inert gas environment.
135) The process of clause 134), wherein the closed vessel is placed inside a secondary packaging, for example a heat-sealable foil pouch, under an inert gas environment.
136) The process of any of clauses 133) to 135), wherein the inert gas is argon (Ar), helium (He), neon (Ne), krypton (Kr), xenon (Xe), and radon (Rn), preferably argon (Ar).
137) A process for producing a lyophilized composition for use as a color control composition in a colorimetric nucleic acid amplification reaction, the process corresponding to the process of any of clauses 108) to 136), but wherein nucleic acid polymerase and/or reverse transcriptase is excluded from the pre-lyophilisation formulation.
138) A lyophilized composition produced by a process of any of clauses 108) to 137).
139) A rehydrated reaction mixture formed by rehydration of a lyophilised composition of any of clauses 1) to 107) or 138).
140) A method for determining the presence of a target nucleic acid in a test sample, the method comprising:
   a. providing a lyophilised composition according any of clauses 1) to 105) or 138), in a first vessel;
   b. rehydrating the lyophilised composition within the first vessel and adding the test sample to the first vessel to provide a rehydrated reaction mixture;
   c. subjecting the rehydrated reaction mixture to conditions suitable to allow nucleic acid amplification to occur if the target nucleic acid is present in the test sample;
   d. determining a positive result for the presence of a target nucleic acid in the test sample if a colorimetric change occurs due to the presence of nucleic acid amplification.
141) The method of clause 140), wherein the test sample is a sample comprising nucleic acid or suspected of comprising nucleic acid in an eluent.
142) The method of clause 140) or 141), wherein the test sample comprises extracted nucleic acids (RNA and/or DNA) from a biological or environmental sample.
143) The method of clause 142), wherein the biological sample is a nasopharyngeal swab, or a blood, a urine, a saliva, a tissue, stool, serum, lymph, semen, sweat, tears, amniotic fluid or wound exudate sample, or any other bodily fluid and the environmental sample is water, such as waste water.
144) The method of any of clauses 140)-143), wherein the target nucleic acid is a nucleic acid sequence from a pathogen, for example SARS-CoV-2, influenza, respiratory syncytial virus (RSV), human rhinovirus (HR), adenovirus or parainfluenza.
145) The method of any of clauses 140)-144), wherein subjecting the rehydrated reaction mixture to conditions suitable for to allow nucleic acid amplification to occur comprises heating the contents of the sample tube (the rehydrated reaction mixture and test sample) to a temperature suitable to achieve nucleic acid amplification, i.e. at which the nucleic acid polymerase enzyme is active, optionally wherein the temperature is about 30-95° C., 50-80° C., 60-72° C., 60-65° C., or 62-64° C., for example, about 63° C.
146) The method of any of clauses 140)-145), wherein the duration of the amplifying step may be about 1 to 150 minutes, at least about 15 minutes, at least about 20 minutes, preferably about 20-40 minutes or 20-30 minutes.
147) The method of any of clauses 140)-146), wherein the nucleic acid amplification reaction is and isothermal nucleic acid amplification method, preferably Loop-Mediated Isothermal Amplification (LAMP).
148) The method of any of clauses 140)-147), wherein rehydration of the lyophilised reaction mixture and addition of the test sample are carried out as a single step, by addition of extracted RNA or DNA within an eluent to lyophilised reaction mixture.
149) The method of any of clauses 140)-148), wherein the method further comprises the steps of
   a′. providing a lyophilised composition according to clause 106) or 107) in a second vessel;
   b′. rehydrating the lyophilised composition within the second vessel and adding test sample to the second vessel to provide a rehydrated reaction mixture; and
   c′. subjecting the rehydrated reaction mixture to the same conditions as in step c.
150) The method of clause 149, further comprising comparing the color of the reaction mixture in the first and second vessels after steps c and c′ and confirming the presence of a target nucleic acid in the test sample if a colorimetric change occurs in the first vessel but not in the second vessel.
151) The method of any of clauses 140)-150), further comprising the steps of
   a″. providing a lyophilised composition according to any one of clauses 1) to 105) in a third vessel, wherein the composition comprises nucleic acid amplification reagents directed to a human control target nucleic acid;
   b″. rehydrating the lyophilised composition within the third vessel and adding the test sample to the third vessel to provide a rehydrated reaction mixture;
   c″. subjecting the rehydrated reaction mixture to the same conditions as in step c; and
   d″. determining a positive result for the presence of a human control target nucleic acid in the sample if a colorimetric change occurs due to the presence of nucleic acid amplification.
152) The method of clause 151), further comprising comparing the color of the reaction mixture in the first, second and third vessels after steps c, c′ and c″ and i) confirming the presence of a target nucleic acid in the sample if a colorimetric change occurs in the first vessel but not in the second vessel or the absence of target nucleic acid in the sample if no colorimetric change occurs in the first vessel; and ii) confirming that the sample contains human DNA if a colorimetric change occurs in the third vessel.
153) The method of any of clauses 140)-152), further comprising the steps of
   a′″. providing a lyophilised composition according to the first aspect in a fourth vessel, wherein the composition comprises nucleic acid amplification reagents directed to an internal control template nucleic acid;
   b′″. rehydrating the lyophilised composition within the fourth vessel and adding test sample to the fourth vessel to provide a rehydrated reaction mixture;
   c′″. subjecting the rehydrated reaction mixture to the same conditions as in step c; and
   d′″. determining the presence of a positive result if a colorimetric change occurs due to the presence of nucleic acid amplification, wherein an internal control template nucleic acid sequence is present within the lyophilised composition or is added in step b′″.
154) The method of clause 153), wherein the lyophilised composition includes an internal control template nucleic acid or wherein the internal control template nucleic acid is added to the test sample.
155) The method of any of clauses 140)-154), wherein any of steps a-a′″, b-b′″, c-c′″ and d-d′″ are carried out concurrently.
156) The method of any of clauses 140)-155), wherein in any of steps b-b′″, rehydration of the lyophilised reaction mixture and addition of the test sample is carried out as a single step, by addition of extracted RNA or DNA within a sample eluent (e.g. water, such as nuclease-free water) to the lyophilised reaction mixture.
157) A method for diagnosing infection by a pathogen in a subject, the method comprising:
   a. providing a lyophilised composition according to any of clauses 1)-105), wherein the lyophilised composition contains primers directed to a target nucleic acid of the pathogen;
   b. rehydrating the lyophilised composition and adding a test sample obtained from the subject to provide a rehydrated reaction mixture;
   c. subjecting the rehydrated reaction mixture to conditions suitable to allow nucleic acid amplification to occur if the target nucleic acid is present in the test sample; and
   d. determining a positive result for the presence of the target nucleic acid in the test sample if a colorimetric change occurs due to the presence of nucleic acid amplification; and
   e. diagnosing infection by the pathogen on the basis of a positive result.
158) A method for treating infection by a pathogen in a subject, the method comprising:
   a. providing a lyophilised composition according to any of clauses 1)-105), wherein the lyophilised composition contains primers directed to a target nucleic acid of the pathogen;
   b. rehydrating the lyophilised composition and adding a test sample from a subject to provide a rehydrated reaction mixture;
   c. subjecting the rehydrated reaction mixture to conditions suitable to allow nucleic acid amplification to occur if the target nucleic acid is present in the sample;
   d. determining a positive result for the presence of the target nucleic acid in the sample if a colorimetric change occurs due to the presence of nucleic acid amplification; and
   e. diagnosing infection by the pathogen in the subject on the basis of a positive result and treating the subject for said infection.
159) The method of clause 157) or 158), wherein the subject is a human subject.
160) The method of any of clauses 157)-159), wherein treatment comprises administering a therapeutic agent to the subject.
161) The method of any of clauses 157)-160), wherein the test sample comprises extracted nucleic acids (RNA and/or DNA) from a biological sample from the patient or, alternatively, a biological sample from the patient is used directly as the test sample, without extraction of nucleic acid.
162) A kit for carrying out a nucleic amplification reaction, the kit comprising a sample vessel having a lyophilised composition of any of clauses 1)-105) present therein.
163) The kit of clause 162), further comprising a sample vessel having a lyophilised composition for use as a color control composition in a colorimetric nucleic acid amplification reaction of clause 106) or 107) present therein.
164) The kit of clause 162) or 163), further comprising a sample vessel having a lyophilised composition of any of clauses 1)-105) present therein, wherein the lyophilised composition comprises nucleic acid amplification reagents components that enable amplification of a target nucleic acid to occur, wherein the target nucleic acid is a human gene.
165) A pre-lyophilisation formulation comprising nucleic acid amplification reagents; a cryoprotectant; a pH adjusting agent; a colorimetric amplification indicator; and water, optionally wherein the pre-lyophilisation formulation is as defined in any of clauses 67)-105).
166) A pre-lyophilisation formulation of clause 165) having components as defined in any of clauses 67)-105), at corresponding relative concentrations, optionally in a more dilute or more concentration form.
167) A composition, process, method or formulation according to any of the preceding clauses, wherein the nucleic acid amplification reaction may preferably be an isothermal nucleic acid amplification method, preferably a Loop-Mediated Isothermal Amplification (LAMP) reaction.

The present invention will now be described by way of reference to the following examples and accompanying drawings which are present for the purposes of illustration only and are not to be construed as being limiting on the invention.

### EXAMPLES

In the following examples, the custom diluent referenced throughout is an aqueous solution prepared using distilled water of: 500 mM KCl, 20 mM MgSO₄, 100 mM (NH₄)₂SO₄, 1% Tween-20 and NaOH added to reach a pH of 8.8 (˜11.5 mM NaOH).

Nuclease-free water was obtained from ThermoFisher (product code 10977035). Mannitol, Ficoll 400, BSA glycerol-free (product code A9418-5G), phenol red (Cat. No. P3532-5G), thymol blue (Cat no. 114545-5G) and eriochrome black T (Cat. No. 858390-100G) were obtained from Sigma (MERCK). MgSO₄ stock solution was obtained from NEB (product code B1003S).

DNA polymerase (Bst WarmStart) and reverse transcriptase (WarmStart RTx) were from New England Biolabs and were glycerol-free reagents. DNA polymerase and reverse transcriptase were provided in a solution of 10 mM Tris-HCl, 50 mM KCl, 1 mM DTT, 0.1 mM EDTA, 0.1% Triton® X-100, pH 7.1 @25° C.

Percentages in the exemplified formulations are % w/v.

Example 1—Wet LAMP Protocol (Provided for reference purposes) A LAMP Primer mix (as an aqueous solution) was prepared consisting of: 2 μM (each) FIP and BIP, 1 μM (each) LF and LB and 0.25 μM (each) F3 and B3.

LAMP reaction formulations of 20 μL final volume were prepared consisting of: 2 μL custom diluent, 2 μL LAMP primer mix, 4 to 10 mM MgSO₄ (from a 100 mM stock), 1-1.8 mM each dNTP (stock 25 mM each), 0-1.2 mg/mL glycerol-free or glycerol-based BSA (stock at 20 mg/mL), 0-0.8 M glycerol-free or glycerol-based betaine (stock at 5 M), 0.5 U/μL Warmstart Bst 2.0 (New England Biolabs). For RNA targets 0.45 U/μL of WarmStart RTx (New England Biolabs) was added. Enough nuclease-free water was added accordingly to the reaction to a final volume of 20 μL. Syto-9 Green Fluorescent Nucleic Acid Stain was added to a concentration of 0.05 μM (ThermoFisher)(stock 20 μM).

This composition was used to perform a LAMP reaction on samples containing 1,000 copies/μL SARS-CoV-2 RNA with incubation at 63° C. for 30-40 min in a LightCycler 96 (Diagnostics Roche).

The primers used correspond to the set 2 primers as described below in example 6.

### Example 2—Colorimetric Wet LAMP Using Indicators (Provided for Reference Purposes)

The suitability of colorimetric indicators for the wet LAMP protocol was assessed by titration of colorimetric indicators. The protocol of example 1 was performed in reactions containing 1,000 copies/μL SARS-CoV-2 RNA. The following indicators were included, each in an independent test reaction: calmagite, xylidyl blue, phenol red, neutral red, eriochrome black T, hydroxynaphtol blue,
thymol blue, and bromothymol blue. A conventional thermal-cycling instrument was used to monitor real-time fluorescence.

Eriochrome black T (EBT) showed the most distinguishable color change. Concentrations can be varied, but a final concentration of 50 μM was optimal for a significant color change without inhibition of amplification. An additional cross-titration was performed by combining each of these dyes with the others to determine if any combination exhibited an enhanced change in color. The combination of eriochrome black T and thymol blue resulted in a color change from red to green.

Lastly, a combined titration of these two dyes was performed in order to establish an optimal composition. A combination of 50 μM eriochrome black T and 125 μM thymol blue performed optimally, with no negative impact on the performance of the LAMP reaction.

### Example 3—Development of an Optimised LAMP Protocol

The protocol of example 1 was further used to investigate the optimum concentration of components for the LAMP reaction.

MgSO₄ was titrated with ranging concentrations between 4 mM to 10 mM. The recommended concentration by the manufacturer (New England Biolabs) is 8 mM (2 mM part of the custom 10× isothermal buffer). All concentrations gave successful LAMP reactions, with reaction time increasing as the concentration of MgSO₄ᵥᵢₛ increased. In this titration, the most optimal concentration was observed as between 6 mM and 7 mM.

dNTPs which are key for the synthesis of DNA, were titrated with concentrations ranging
between 1 mM to 1.8 mM. All concentrations gave successful LAMP reactions. In this titration, the most optimal concentration was observed as 1.4 mM.

The addition of Bovine Serum Albumin (BSA) and Betaine was investigated. The incorporation of BSA is not recommended by the manufacturer of the polymerase enzymes, however it was found that the addition slightly improved assay reproducibility (lower standard deviation) and speed of the reaction. BSA at a concentration of 1.2 mg/mL showed the best performance, and results were improved further with the glycerol-free version. It was found that glycerol had a negative effect on the speed of the reaction.

Three alternative DNA polymerase enzymes were investigated using the protocol of example 1: Bst 2.0 DNA polymerase, Bst 2.0 WarmStart DNA polymerase and Bst 3.0 DNA polymerase. It was found that the optimised conditions described above led to a significantly faster reaction in comparison to that obtained when utilising the conditions recommended by the manufacturer. It was further found that Bst 2.0 WarmStart DNA polymerase had the best performance in terms of LAMP reaction catalysis.

### Example 4—Development of Lyophilised LAMP

LAMP protocols were subjected to freeze-drying microscopy (FDM) at varying compositions in order to achieve a lyophilised reaction mix that could be used for a LAMP reaction upon rehydration. Cryoprotectant components were also included to promote stability of the lyophilised composition. Aliquots of LAMP reaction mixture were dispensed to form beads of 20 μL volume. The critical temperature obtained from freeze drying microscope (FDM) analysis was used to design a freeze-drying protocol. The process of lyophilisation was carried out using either a Virtus Genesis or a SP VirTis Ultra freeze dryer.

Lyophilisation was carried out by applying the conditions in table 1 below

TABLE 1

| | Temperature | Step | Time | Vaccuum |
|---|---|---|---|---|
| Phase | (° C.) | (Ramp/Hold) | (min) | (mTorr) |
| Pre- | −45 | R | 30 | NA |
| cooling | −45 | H | 30 | NA |
| Primary | −40 | R | 60 | 60 |
| drying | −40 | H | 1100 | 60 |
| | −40 | H | 1000 | 60 |
| Secondary | +20 | R | 120 | 60 |
| drying | +20 | H | 360 | 60 |

Successful lyophilization was achieved with the cryoprotectants being either a mixture of mannitol and ficoll-400, or a mixture of trehalose and dextran-70 at the concentrations indicated in Table 2 below.

TABLE 2

| | Optimised concentration in | |
|---|---|---|
| Reagent | LAMP reaction mixture | |
| Mannitol | 4 | % w/v |
| Ficoll-400 | 2 | % w/v |
| Trehalose | 4 | % w/v |
| Dextran 70 | 4 | % w/v |

As mentioned above, the critical temperature obtained from freeze drying microscope (FDM) analysis was used to design a freeze-drying protocol. For example, using a lyostat FDM, the onset of collapse (Tc or Toc) was determined at −38.6° C. and the total collapse (Tfc) at −37.9° C. under a pressure of 5.4 Pa.

A comparison was carried out between the wet LAMP formulation and a lyophilised LAMP formulation. The wet LAMP formulation was prepared according to the in Table 3 protocol and then used to perform a LAMP reaction.

TABLE 3

| | | | | | Volume per |
|---|---|---|---|---|---|
| | | | | | Rx (uL) |
| Component | Stock conc. | | Final conc. | | 10 ul |
| Nuclease-free Water | | | | | 0.74 |
| Custom diluent | 10 | x | 1 | x | 1.00 |
| Mannitol | 20 | % | 4 | % | 2.00 |
| Ficoll | 20 | % | 2 | % | 1.00 |
| MgSO₄ | 100 | mM | 5 | mM | 0.50 |
| dNTPs | 25 | mM | 1.4 | mM | 0.56 |
| BSA glycerol-free | 20 | mg/mL | 1.2 | mg/mL | 0.60 |
| 10X LAMP | 10 | x | 1 | x | 1.00 |
| NaOH | 0.2 | M | 0.005 | M | 0.25 |
| Bst 2.0 WarmStart | 120 | U/ul | 0.5 | U/ul | 0.04 |
| glycerol-free | | | | | |
| Syto9 | 20 | uM | 0.5 | uM | 0.25 |
| RNA (added later | | copies/ul | | copies/ul | 2.00 |
| for LAMP reaction, | | | | | |
| at varying | | | | | |
| concentrations) | | | | | |
| RTx WarmStart | 75 | U/uL | 0.45 | U/uL | 0.06 |
| glycerol-free | | | | | |

The lyophilised formulation was prepared as described above, according to the following protocol in Table 4:

TABLE 4

| | | | | | Volume per |
|---|---|---|---|---|---|
| | | | | | Rx (uL) |
| Component | Stock conc. | | Final conc. | | 10 ul |
| Nuclease-free Water | | | | | 3.74 |
| Custom diluent | 10 | x | 1 | x | 1.00 |
| Mannitol | 20 | % | 4 | % | 2.00 |
| Ficoll | 20 | % | 2 | % | 1.00 |
| MgSO₄ | 100 | mM | 5 | mM | 0.50 |
| dNTPs | 25 | mM | 1.4 | mM | 0.56 |
| BSA glycerol-free | 20 | mg/mL | 1.2 | mg/mL | 0.60 |
| NaOH | 0.2 | M | 0.005 | M | 0.25 |
| Bst 2.0 WarmStart | 120 | U/ul | 0.5 | U/ul | 0.04 |
| glycerol-free | | | | | |
| Syto9 | 20 | uM | 0.5 | uM | 0.25 |
| RTx WarmStart | 75 | U/uL | 0.45 | U/uL | 0.06 |
| glycerol-free | | | | | |

Aliquots of this were subject to lyophilization. A rehydration mixture was prepared containing nuclease-free water (7 parts), LAMP primer mix (1 part, as in example 1) and SARS-CoV-2 RNA (2 parts, at variable concentrations) The lyophilised beads were rehydrated using this mixture and LAMP reactions were performed.

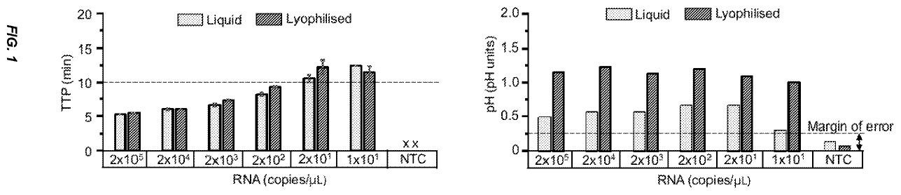

FIG. 1 summarises results observed, showing (A) analytical sensitivity of the lyophilised LAMP beads and the wet LAMP protocol using a serial 10-fold dilution of SARS-CoV-2 RNA and a tandem-LAMPcov assay and (B) a comparison of the ΔpH obtained after amplification of lyophilised LAMP beads and the wet LAMP protocol (liquid) using a serial 10-fold dilution of SARS-CoV-2 RNA and a tandem-LAMPcov assay. The results shown in FIG. 1 demonstrated that the lyophilised format resulted in a higher pH change for the amplification reaction and faster speed at the lowest RNA concentration.

### Example 5—Optimisation of pH Conditions for Colorimetric Lyophilised Format

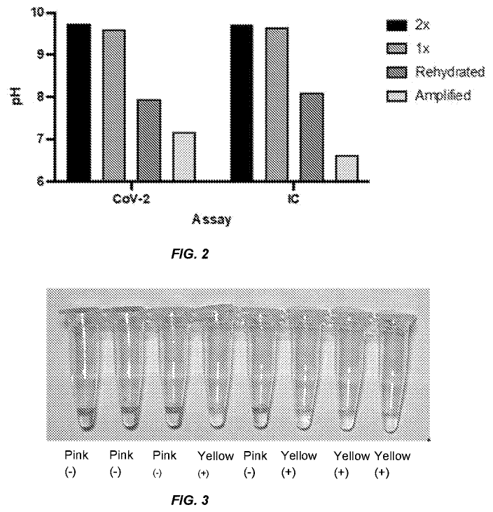

To assess the effect of the lyophilization process on pH, lyophilised cakes were produced from 10 μL pre-lyophilisation LAMP mixtures containing 17.5 mM NaOH, which were subsequently rehydrated in 20 μL of water (i.e. the lyophilized cakes are produced at a 2× concentration). Thus, the pre-lyophilisation mixture can be considered to have a concentration of 2×, while the post-lyophilised mixture has concentration 1×. In order to determine the pH loss that occurs during the freeze-drying process, the pH of the pre-lyophilisation mixture, the pre-lyophilisation mixture diluted to a 1× concentration, and the rehydrated cake pre (rehydrated) and post-amplification (amplified) were measured. These measurements were performed for both the cake containing SARS CoV-2 primer sets and the cake containing the internal control (IC) primer sets and DNA. Results are shown in FIG. 2.

The 2× pre-lyophilisation mix had a pH of 9.73 which remained high upon diluting the mixture to 1×. There was a net loss, amounting to around 1.77 units of pH during the lyophilisation process (from pH 9.73 in the 2×-pre lyophilised mix to a pH of 7.97 in the rehydrated mix). During amplification, pH reduced further to around 6.84 units, a decrease of 1.12 pH units from the rehydrated mix, indicative of a successful LAMP reaction.

When increasing the pH of the wet LAMP reaction to match that of the pre-lyophilisation mix, the addition of ≥7 mM NaOH resulted in a pH of 9.77. At this sodium hydroxide concentration, the reaction was significantly inhibited in both real-time fluorescence and change in pH between pre- and post-amplification pH. This pH experiment showed that the pH of the pre-lyophilisation mix (17.5 mM NaOH) recreated in a wet reaction results in pH that is too high to give appropriate pH changes on amplification. In this wet LAMP format no significant change in pH was achieved in reactions containing >10 mM NaOH. Furthermore, in the presence of the pH-sensitive dye, phenol red, these highly alkaline solutions did not display a significant change in color from positive amplification, above a pH 9.0 (6 mM NaOH) indicative that the LAMP reaction was outside the optimal dynamic range of this dye.

### Example 6—Use of the Optimised Colorimetric LAMP Reaction for the Detection of SARS-Cov-2 RNA with pH Indicator

### Step 1—Pre-Lyophilisation Formulation

### Preparation of Nucleic Acid Amplification Reagents

Various pre-lyophilisation formulations were prepared as described below to provide composition for use for color, human and internal controls and for testing samples as outlined below. The various pre-lyophilisation formulations were dispensed into sample tube strips and subjected to lyophilisation before then being used to perform a colorimetric LAMP reaction.

Prior to use, reagents were stored at −20° C., −80° C. or 4° C. as appropriate (mannitol, ficoll, trehalose, dextran and indicators stored at 4° C., enzymes stored at −80° C., other reagents stored at −20° C.). All DNA primer mixes and solutions were purchased from Integrated NDA Technologies (IDT) or Thermofisher Scientific in dry format and rehydrated to 400 μM in nuclease-free water. LAMP reaction mixture comprised combinations of primers as detailed herein.

### LAMP Reaction Mixture A—Color Control

A pre-lyophilisation formulation was prepared containing the following: 200 μL of solution B, 200 μL Mannitol (stock at 20% w/v), 100 μL Ficoll type 400 (stock at 20% w/v), 100 μL MgSO₄ (stock at 100 mM), 35 μL NaOH (stock at 500 mM), 112 μL dNTP mix (stock at 25 mM, Invitrogen (ThermoFisher)), 60 μL bovine serum albumin (stock at 40 mg/mL, glycerol-free, Sigma (MERCK)), 4 μL phenol red (stock at 25000 μM), 33 μL DNA primer solution as further detailed below and sufficient nuclease-free water to bring the total volume to 1000 μL. Stock refers to aqueous stock solutions at the recited concentrations.

The DNA primer solution (COL CTRL-PM) contained 16.25 μL CytoB_B3 (stock at 400 μM) and 16.25 μL CytoB_BIP (stock at 400 μM), as shown in Table 5.

TABLE 5

| Primer | Protocol | |
|---|---|---|
| name | name | Sequence |
| CytoB_ | IC_B3 | GTTTGCATGTAGATAGCGAAT |
| B3 | | (SEQ ID NO: 1) |
| CytoB_ | IC_BIP | AGCAATACATTACACATCAGA |
| BIP | | CACATTTTATCCGTAATTT |
| | | ACGTCTCGA (SEQ ID NO: 2) |

This pre-lyophilisation formulation is summarised in Table 6:

TABLE 7

| | | | | | Volume in |
|---|---|---|---|---|---|
| | | | Final conc. in pre- | | μL per |
| | Stock conc. | | lyophilisation | | 10 μL |
| Reagent | used | | formulation | | reaction |
| Nuclease-free Water | | | | | 1.56 |
| Solution B | 10 | x | 2 | x | 2 |
| Mannitol | 20 | % | 4 | % | 2.00 |
| Ficoll | 20 | % | 2 | % | 1.00 |
| MgSO₄ | 100 | mM | 10 | mM | 1.00 |
| NaOH | 500 | mM | 17.5 | mM | 0.35 |
| dNTP mix | 25 | mM | 2.8 | mM | 1.12 |
| BSA glycerol-free | 40 | mg/mL | 2.4 | mg/mL | 0.60 |
| Phenol Red | 25000 | uM | 100 | uM | 0.04 |
| COL CTRL-PM | | | | | 0.33 |
| TOTAL | | | | | 10 |

The color control LAMP reaction mixture does not certain reaction chemistry elements (DNA polymerase and reverse transcriptase) to prevent amplification even in the presence of environmental contamination or contamination from adjacent tubes. It is used as a reference to which the other tube colors are compared. The resuspended pH is identical to that of the other sample tubes in the strip. Accordingly, the color control also acts as an adjusting baseline that changes slightly depending on the pH of the input sample.

### LAMP Reaction Mixture B—SARS-CoV-2 RNA Detection

A pre-lyophilisation formulation was prepared containing the following: 200 μL of custom diluent, 200 μL Mannitol (stock at 200% w/v), 100 μL Ficoll type 400 (stock at 200% w/v), 100 μL MgSO₄ (stock at 100 mM), 35 μL NaOH (stock at 500 mM), 112 μL dNTP mix (stock at 25 mM, Invitrogen (ThermoFisher)), 60 μL bovine serum albumin (stock at 40 mg/mL, glycerol-free, Sigma (MERCK)), 4 μL phenol red (stock at 25000 μM), 8.3 μL Bst 2.0 DNA Polymerase (New England Biolabs, UK) (stock at 120 U/μL), 12 μL of RTx reverse transcriptase (New England Biolabs, UK) (stock at 75 U/μL), 65 μL DNA primer solution as further detailed below and sufficient nuclease-free water to bring the total volume to 1000 μL.

The DNA primer solution contained the primers as shown in Table 8:

TABLE 8

| Primer | Protocol | | Conc. |
|---|---|---|---|
| name | name | Sequence | (μM) |
| F3_Set 1 | CoV1- | TTG TGA TCA ACT CCG CGA A (SEQ ID NO: 3) | 0.5 |
| | F3 | | |
| B3_Set 1 | CoV1- | TCA ATT AAA TTG TCA TCT TCG TCC T (SEQ ID NO: | 0.5 |
| | B3 | 4) | |
| LF_Set 1 | CoV1- | CTG CAC TTA CAC CGC AAA C (SEQ ID NO: 5) | 2 |
| | LF | | |
| LB_Set 1 | CoV1- | ATG ATA AAG TAG CTG GTT TTG CT (SEQ ID NO: 6) | 2 |
| | LB | | |
| FIP_Set 1 | CoV1- | TCA GTA CTA GTG CCT GTG CCG TCA GCT GAT GCA | 4 |
| | FIP | CAA TCG TT (SEQ ID NO: 7) | |
| BIP_Set 1 | CoV1- | TGT CGT ATA CAG GGC TTT TGA CAT CTT GGA AGC | 4 |
| | BIP | GAC AAC AAT TAG T (SEQ ID NO: 8) | |
| F3_Set 2 | CoV2- | ACC AAT AGC AGT CCA GAT GA (SEQ ID NO: 9) | 0.5 |
| | F3 | | |
| B3_Set 2 | CoV2- | CAC GAT TGC AGC ATT GTT AGC (SEQ ID NO: 10) | 0.5 |
| | B3 | | |
| LF_Set 2 | CoV2- | GGA CTG AGA TCT TTC ATT TTA CCG T | 2 |
| | LF | (SEQ ID NO: 11) | |
| LB_Set 2 | CoV2- | ACT GAG GGA GCC TTG AAT ACA (SEQ ID NO: 12) | 2 |
| | LB | | |
| FIP_Set 2 | CoV2- | TCTGGCCCAGTTCCTAGGTAGTCCAGACAAATTCGTG | 4 |
| | FIP | GTGG (SEQ ID NO: 13) | |
| BIP_Set 2 | CoV2- | GGACTTCCCTATGGTGCTAACAAACGGGTGCCAATGT | 4 |
| | BIP | GATCT (SEQ ID NO: 14) | |

This pre-lyophilisation formulation is summarised in Table 9:

TABLE 9

| | | | | | Volume in |
|---|---|---|---|---|---|
| | | | Final conc. in pre- | | μL per |
| | Stock conc. | | lyophilisation | | 10 μL |
| Reagent | used | | formulation | | reaction |
| Nuclease-free Water | | | | | 1.04 |
| Solution B | 10 | x | 2 | x | 2 |
| Mannitol | 20 | % | 4 | % | 2.00 |
| Ficoll | 20 | % | 2 | % | 1.00 |
| MgSO₄ | 100 | mM | 10 | mM | 1.00 |
| NaOH | 500 | mM | 17.5 | mM | 0.35 |
| dNTP mix | 25 | mM | 2.8 | mM | 1.12 |
| BSA glycerol-free | 40 | mg/mL | 2.4 | mg/mL | 0.60 |
| Phenol Red | 25000 | uM | 100 | uM | 0.04 |
| Bst 2.0 WarmStart | 120 | U/uL | 1 | U/uL | 0.08 |
| RTx Warmstart | 75 | U/uL | 0.9 | U/uL | 0.12 |
| CoV1 PM | | | | | 0.33 |
| CoV2-PM | | | | | 0.33 |
| TOTAL | | | | | 10 |

### LAMP Reaction Mixture C—Human Control

A pre-lyophilisation formulation was prepared containing the following: 200 μL of custom diluent, 200 μL Mannitol (stock at 20% w/v), 100 μL Ficoll type 400 (stock at 20% w/v), 100 μL MgSO₄ (stock at 100 mM), 35 μL NaOH (stock at 500 mM), 112 μL dNTP mix (stock at 25 mM, Invitrogen (ThermoFisher)), 60 μL bovine serum albumin (stock at 40 mg/mL, glycerol-free, Sigma (MERCK)), 4 μL phenol red (stock at 25000 μM), 8.3 μL Bst 2.0 DNA Polymerase (New England Biolabs, UK) (stock at 120 U/μL), 66 μL DNA primer solution as further detailed below and sufficient nuclease-free water to bring the total volume to 1000 μL.

The DNA primer solution included CTRL1 primers to GAPDH, as shown in Table 10:

TABLE 10

| Primer | Protocol | | Conc. |
|---|---|---|---|
| name | name | Sequence | (μM) |
| GAPDH ID: | CTRL2- | GATGCTGGCGCTGAGTAC | 0.5 |
| 3_F3 | F3 | (SEQ ID NO: 15) | |
| GAPDH ID: | CTRL2- | GCTAAGCAGTTGGTGGTGC | 0.5 |
| 3_B3 | B3 | (SEQ ID NO: 16) | |
| GAPDH ID: | CTRL2- | CTTTTGGCTCCCCCCTGCA | 2 |
| 3_FIP | LF | AATGGAGTCCACTGGCGTC | |
| | | TT (SEQ ID NO: 17) | |
| GAPDH ID: | CTRL2- | TCTGCTGATGCCCCCATGTT | 2 |
| 3_BIP | LB | CGGAGGCATTGCTGATGATC | |
| | | T (SEQ ID NO: 18) | |
| GAPDH ID: | CTRL2- | AGCCTTCTCCATGGTGGTG | 4 |
| 3_LF | FIP | (SEQ ID NO: 19) | |
| GAPDH ID: | CTRL2- | GTCATGGGTGTGAACCATGAG | 4 |
| 3_LB | BIP | (SEQ ID NO: 20) | |

F3, B3, LF, LB, VIP and BIP primers at corresponding concentrations were also be included to a second human gene. The human control could, however, be performed with just a single set of primers.

This pre-lyophilisation formulation is summarised in Table 11:

TABLE 11

| | | | | | Volume in |
|---|---|---|---|---|---|
| | | | Final conc. in pre- | | μL per |
| | Stock conc. | | lyophilisation | | 10 μL |
| Reagent | used | | formulation | | reaction |
| Nuclease-free Water | | | | | 1.15 |
| Solution B | 10 | x | 2 | x | 2 |
| Mannitol | 20 | % | 4 | % | 2.00 |
| Ficoll | 20 | % | 2 | % | 1.00 |
| MgSO₄ | 100 | mM | 10 | mM | 1.00 |
| NaOH | 500 | mM | 17.5 | mM | 0.35 |
| dNTP mix | 25 | mM | 2.8 | mM | 1.12 |
| BSA glycerol-free | 40 | mg/mL | 2.4 | mg/mL | 0.60 |
| Phenol Red | 25000 | uM | 100 | uM | 0.04 |
| Bst 2.0 | 120 | U/uL | 1 | U/uL | 0.08 |
| WarmStart | | | | | |
| CTRL1-PM | | | | | 0.33 |
| CTRL2-PM | | | | | 0.33 |
| TOTAL | | | | | 10 |

The human control serves two purposes. First, it confirms that an adequate sample was taken from the patient. Second, it confirms the sample preparation process was performed correctly. The absence of a colorimetric change in the color control, would indicate that human DNA is not present in the sample elution, and that either sample acquisition or sample preparation failed. The test in that case would be considered invalid.

### LAMP Reaction Mixture D—Internal Control

A pre-lyophilisation formulation was prepared containing the following: 200 μL of custom diluent, 200 μL Mannitol (stock at 200% w/v), 100 μL Ficoll type 400 (stock at 200% w/v), 100 μL MgSO₄ (stock at 100 mM), 35 μL NaOH (stock at 500 mM), 112 μL dNTP mix (stock at 25 mM, Invitrogen (ThermoFisher)), 60 μL bovine serum albumin (stock at 40 mg/mL, glycerol-free, Sigma (MERCK)), 4 μL phenol red (stock at 25000 μM), 8.3 μL Bst 2.0 DNA Polymerase (New England Biolabs, UK) (stock at 120 U/μL), 33 μL DNA primer solution (IC_PM) as further detailed below, 10 μM internal control template and sufficient nuclease-free water to bring the total volume to 1000 μL.

This pre-lyophilisation formulation is summarised in Table 12:

TABLE 12

| | | | | | Volume in |
|---|---|---|---|---|---|
| | | | Final conc. in pre- | | μL per |
| | Stock conc. | | lyophilisation | | 10 μL |
| Reagent | used | | formulation | | reaction |
| Nuclease-free | | | | | 1.38 |
| Water | | | | | |
| Solution B | 10 | x | 2 | x | 2 |
| Mannitol | 20 | % | 4 | % | 2.00 |
| Ficoll | 20 | % | 2 | % | 1.00 |
| MgSO₄ | 100 | mM | 10 | mM | 1.00 |
| NaOH | 500 | mM | 17.5 | mM | 0.35 |
| dNTP mix | 25 | mM | 2.8 | mM | 1.12 |
| BSA glycerol- | 40 | mg/mL | 2.4 | mg/mL | 0.60 |
| free | | | | | |
| Phenol Red | 25000 | uM | 100 | uM | 0.04 |
| Bst 2.0 | 120 | U/uL | 1 | U/uL | 0.08 |
| WarmStart | | | | | |
| IC_PM | 400 | uM | | uM | 0.33 |
| IC_template | 1.00E+04 | copies/uL | 100 | copies/uL | 0.10 |
| Total | | | | | 10 |

The DNA primer solution contained the primers as shown in Table 13:

TABLE 13

| Primer | Protocol | | Conc. |
|---|---|---|---|
| name | name | Sequence | (μM) |
| CytoB_ | IC_F3 | ATTCATTGACCTCCCAGC | 0.5 |
| F3 | | (SEQ ID NO: 21) | |
| CytoB_ | IC_B3 | GTTTGCATGTAGATAGCG | 0.5 |
| B3 | | AAT (SEQ ID NO: 1) | |
| CytoB_ | IC_LF | CAAGATTAGGCAGATGCCT | 2 |
| LF | | AAGAG (SEQ ID NO: 22) | |
| CytoB_ | IC_LB | ACAACAGCTTTCTCATCAGT | 2 |
| LB | | TACAC (SEQ ID NO: 23) | |
| CytoB_ | IC_FIP | AAGAACAGGCCTGTTAGGA | 4 |
| FIP | | TTTGTTTTATCATGATGA | |
| | | AACTTCGGTTC | |
| | | (SEQ ID NO: 24) | |
| CytoB_ | IC_BIP | AGCAATACATTACACATCA | 4 |
| BIP | | GACACATTTTATCCGTAAT | |
| | | TTACGTCTCGA | |
| | | (SEQ ID NO: 2) | |

The internal control template, a synthetic DNA including a sequence based on portion of a porcine cytochrome B gene, had the following sequence:

(SEQ ID NO: 25)
TCAACAACGCATTCATTGACCTCCCAGCCCCCTCAAACATCTCATC
ATGATGAAACTTCGGTTCCCTCTTAGGCATCTGCCTAATCTTGCA
AATCCTAACAGGCCTGTTCTTAGCAATACATTACACATCAGACAC
AACAACAGCTTTCTCATCAGTTACACACATTTGTCGAGACGTAAA
TTACGGATGAGTTATTCGCTATCTACATGCAAACGGAGCATCCAT
ATTCTTTATTTGCCTATTCATCCACGTAGGCCGAGGTCTATACTA
CGGATCCTATATATTCCTAGAAACATGAAACATTGGAGTAGTCCT
ACTATTTACCGTTATAGCAACAGCCTTCATAGGCTACGTCCTGCC
CTGAGGACAAATATCATTCTGAGGAGCTACGGTCATCACAAATCT
ACTATCAGCTATCCCTTATATCGGAACAGACCTCGTAGAATGAAT
CTGAGGGGGCTTTTCCGTCGACAAAGCAACCCTCACACGATTCTT
CGCCTTCCACTTTATCCTGCCATTCATCATTACCGCCCTCGCAGC
CGTACATCTCCTATTCCTGCACGAAACCGGATCCAACAACCCTAC
CGGAACATGTACATATTATTATTAATATTACATAGTACATATCAT
TATTGATCGTACATAGCACATATCATGTCAAATAACTCCAGTCAA
CATGCGTATCACCACCATTAGATCACGAGCTTAATTACCATGCCG
CGTGAAACCAGCAACCCGCTTGGCAGGGATCCCTCTTCTCGCTCC
GGGCCCATAAATCGTGGGGGTTTCTATTGATGAACTTTAACAGGC
ATCTGGTTCTTACTTCAGGACCATCTCATCTAAAATCGCCCACTC
TTTCCCCTTAAATAAGACATCTCGATGGACTAATGACTAATCAGC
CCATGCTCACACATAACTGAGGTTTCATACATTTGGTATTTTTTA
ATTTTTGGGGATGCTTGGACTCAGCCATGGCCGTCAAAGGCCCTA
ACACAGTCAAATCAATTGTAGCTGGACTTCATGGAACTCATGATC
CGGCACGACAATCCAAACAAGGTGCTATTCAGTCAATGGTTACGG
GACATAACGTGCGTACACGCGCATATAAGCAGGTAAATTATTAGC
TCATTCAAACCCCCCTTACCCCCCATTAAACTTATGCTCTACACA
CCCTATAACGCCTTGCCAAACCCCCCGGTGAGAATGCCCTCCAGA
TCCTAAAGATCAAAAGGAGCAGGTATCAAGCACACCTATAACGGT
AGCTCATAACGCCTTGCTCAACCACACCCCCACGGGAAACAGCAG
TGATAAAAATTAAGCCATGAACGAAAGTTTGACTAAGTTATATTA
ATTAGAGTTGGTAAATCTCGTGCCAGCCACCGCGGTCATACGATT
AACCCAAATTAATAGATCCACGGCGTAAAGAGTGTTTAAGAAAAA
AAAACCACAATAGAGTTAAATTATAACTAAGCTGTAAAAAGCCCT
AGTTAAAATAAAATAACCCACGAAAGTGACTCTAATAATCCTGAC
ACACGATAGCTAGGACCCAAACTGGGATTAGATACCCCACTATGC
CTAGCCCTAAACCCAAATAGTTACATAACAAAACTATTCGCCAGA
GTACTACTCGCAACTGCCTAAAACTCAAAGGACTTGGCGGTGCTT
CACATCCACCTAGAGGAGCCTGTTCTATAATCGATAAACCCCGAT
AGACCTTACCAACCCTTGCCAATTCAGCCTATATACCGCCATCTT
CAGCAAACCCTAAAAAGGAACAATAGTAAGCACAATC

The internal control contains primers and their respective template DNA in the mixture for lyophilisation from the outset. This can be used to check for three things. First, that reagents have been stored correctly and not damaged at any stage prior to or while running a LAMP reaction. Second, that the heater was at the correct temperature (and incubated long enough) for the LAMP reaction to occur. Third, that an extracted sample is free from significant inhibitory contaminants. If all three conditions are satisfied, nucleic acid amplification will occur giving rise to a colorimetric change, and the result is valid.

### Step 2—Lyophilisation

The pre-lyophilisation formulations were then dispensed in 10 μL aliquots into to 8-tube strips of 200 μL capacity surrounded by ice packs.

The 8 tubes in each strip contained formulations in the following sequence:
Tube 1: Color control (A)
Tube 2: Color control (A)
Tube 3: Color control (A)
Tube 4: SARS-Cov-2 detection (B)
Tube 5: SARS-Cov-2 detection (B)
Tube 6: Human control (C)
Tube 7: Internal control (D)
Tube 8: Internal control (D)

The strips were arranged in 96-well plates and these were placed in a metallic tray in preparation for subsequent freeze-drying. All strips were open to air.

The pre-lyophilisation formulations were then lyophilised by subjecting the samples to the conditions in table 14.

TABLE 14

| | | | Shelf | | |
|---|---|---|---|---|---|
| | | | temper- | | |
| | | Descrip- | ature | Pressure | Time |
| Stage | Step | tion | (° C.) | (μBar) | (minutes) |
| Flash freeze | 1 | Hold | −50 | Atmospheric | 120 |
| Annealing | 2 | Ramp | −15 | Atmospheric | 70 |
| | 0 | Hold | −15 | Atmospheric | 120 |
| | 4 | Ramp | −50 | Atmospheric | 70 |
| | 5 | Hold | −50 | Atmospheric | 60 |
| Primary drying | 6 | Hold | −50 | 20 | 2980 |
| Secondary | 7 | Ramp | 20 | 20 | 150 |
| drying | 8 | Hold | 20 | 20 | 360 |
| | 9 | Aerate with | 20 | Atmospheric | N/A |
| | | N₂ | | | |

The freeze temperature in the lyophilisation process was −50° C., with a condenser set point of −60° C. The vacuum set point was 500 mTorr. The presence of the ficoll 400 and mannitol facilitated the lyophilisation process. The collapse temperature was determined to be −44.4° C.

### Step 3—RNA/DNA Sample Extraction and Rehydration

After the lyophilisation process, lyophilised cakes were obtained. For the validation of the performance of the cakes, nasopharyngeal samples were collected from healthy volunteers using sterile flocked swabs wherein the samples were stored in a guanidine-thiocyanate medium (eNat, Copan Diagnostics, USA). DNA/RNA extraction was then performed. The protocol for DNA/RNA extraction used was QIAGEN QIAamp viral RNA mini kit following the manufacturer's instructions. The elution buffer was substituted with nuclease-free water.

Other DNA/RNA extraction techniques may be used, including those utilising magnetic beads. Principles of magnetic bead extraction are well established and outlined for example in U.S. Pat. Nos. 4,554,088 and 5,705,628, which themselves reference further background art in respect of this technique. By means of example, a process of extraction using magnetic bead and an elution liquid (nuclease free water), comprising steps as follows has been found to be suitable:
(1) Tube 1 preloaded with lysis/binding buffer (300 uL) (based on guanidinium thiocyanate), IPA (400 uL) and magnetic beads (20 uL), where 400 uL of sample inactivated in eNAT were added. Mix for 30 sec-1 min.
(2) Magnetic beads transferred to Tube 2 containing wash solution (300 uL of 80% ethanol). Mix by 30 sec-1 min.
(3) Magnetic beads transferred to Tube 3 containing 200 uL of nuclease-free water. Mix by 30 sec-1 min. Magnetic beads were discarded.

Elution from the DNA/RNA extraction was split into portions and spiked with the relevant target to assess in order to validate the specificity of the lyophilised compositions. For example, in the case of the SARS-Cov-2 detection compositions, SARS-CoV-2 RNA from Twist Bioscience was spiked in the elution.

The 20 uL of extracted DNA/RNA elution was dispensed into each of the tubes of the 8-tube strips therefore rehydrating the lyophilised composition (to a final concentration of 1×, where the pre-lyophilisation formulation is taken to have a concentration of 2×). The rehydrated composition had a pH of 7.96.

### Step 4—LAMP Reaction and Assessment of Result of Test

After rehydration, the 8-tube strip was placed into a thermal block at 63° C. for 25-35 min.

A pH change was recorded as from the starting pH of 7.96 to an end pH of 6.84 for the SARS-CoV-2 LAMP reaction.

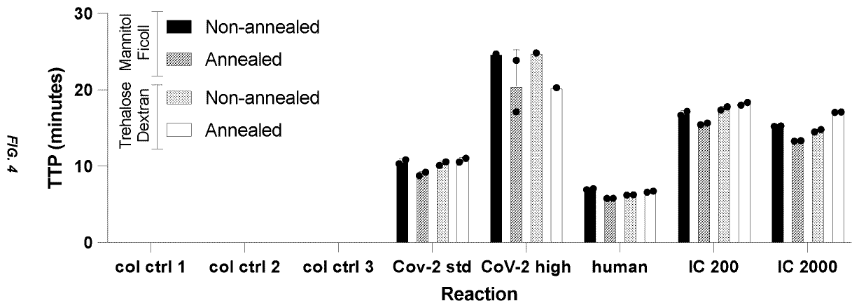

The resulting colors for each tube in the strip are shown in FIG. 3. Tubes 4, 6, 7 and 8 changed color from pink to yellow (a positive result) while the remaining tubes remained pink. This indicates that the LAMP reaction has occurred only in tubes 4, 6, 7 and 8.

The result is interpreted as follows. Tubes 1 to 3, containing color control composition, showed no color change and provide a baseline result. A positive reaction in tubes 7 and 8 (internal control) confirms that the LAMP reaction mixture components have survived the lyophilisation process and thus that the assay is working. The reaction in tube 6 (human control) confirms that the patient sample is of sufficient quality to allow detection of the pathogen of interest. Finally, the positive reaction in tube 4 (SARS-Cov-2 containing patient sample) confirms the preference of the SARS-CoV-2 RNA and thus a positive diagnosis can be made.

To summarize, the human control and internal control should always be positive to determine a valid test. The human control targets a human gene and is used to validate the successful extraction of the sample and its quality (not degraded). The internal control consists of a non-human assay and also includes its respective non-human template and therefore this is used to validate that the reagents are working as expected, for example with the lyophilised compositions being stable at the stored conditions.

### Example 7—Detection of SARS-Cov-2 RNA with Mixed Indicators

The method described in Example 6 utilising a pH indicator was applied mutatis mutandis in the design of an assay utilising a mixture of indicators comprising a pH indicator and a metal ion indicator.

A pre-lyophilisation formulation was prepared containing the following: 200 μL of custom diluent, 200 μL Mannitol (stock at 20% w/v), 100 μL Ficoll type 400 (stock at 20% w/v), 100 μL MgSO₄ (stock at 100 mM), 19 μL NaOH (stock at 500 mM), 112 μL dNTP mix (stock at 25 mM, Invitrogen (ThermoFisher)), 60 μL bovine serum albumin (stock at 40 mg/mL, glycerol-free, Sigma (MERCK)), 4 μL eriochrome black T (stock at 25000 μM), 10 μL thymol blue (stock at 25000 μM), 8.3 μL Bst 2.0 DNA Polymerase (New England Biolabs, UK) (stock at 120 U/μL), 12 μL of RTx reverse transcriptase (New England Biolabs, UK) (stock at 75 U/μL), 66 μL DNA primer solution as further detailed below, and sufficient nuclease-free water to bring the total volume to 1000 μL.

The DNA primer solution (PM) contained the same primers as in ‘LAMP Reaction Mixture B—SARS-CoV-2 RNA detection’ of Example 6:

This pre-lyophilisation formulation is summarised in Table 15:

TABLE 15

| | | | Final conc. in pre- | | Volume per |
|---|---|---|---|---|---|
| | | | lyophilisation | | rxn (μL) |
| Reagent | Stock conc. | | formulation (2x) | | 10 μL |
| Nuclease-free | | | | | 1.10 |
| Water | | | | | |
| Custom buffer | 10 | x | 2 | x | 2 |
| Mannitol | 20 | % | 4 | % | 2.00 |
| Ficoll | 20 | % | 2 | % | 1.00 |
| MgSO4 | 100 | mM | 10 | mM | 1.00 |
| NaOH | 500 | mM | 9.5 | mM | 0.19 |
| dNTP mix | 25 | mM | 2.8 | mM | 1.12 |
| BSA glycerol-free | 40 | mg/mL | 2.4 | mg/mL | 0.60 |
| Eriochrome | 25000 | uM | 100 | uM | 0.04 |
| Black T | | | | | |
| Thymol Blue | 25000 | uM | 250 | uM | 0.10 |
| Bst 2.0 | 120 | U/uL | 1 | U/uL | 0.08 |
| WarmStart | | | | | |
| RTx Warmstart | 75 | U/uL | 0.9 | U/uL | 0.12 |
| CoV1 PM | | | | | 0.33 |
| CoV2-PM | | | | | 0.33 |

As in example 6, 10 μL aliquots of pre-lyophilisation formulation were subjected to lyophilisation. Here, the lyophilisation protocol was as shown in table 16.

TABLE 16

| | | | Shelf | | |
|---|---|---|---|---|---|
| | | | temper- | | |
| | | Descrip- | ature | Pressure | Time |
| Stage | Step | tion | (° C.) | (μBar) | (minutes) |
| Flash freeze | 1 | Loading | −45 | Atmospheric | 120 |
| Primary drying | 2 | Hold | −40 | 60 | 30 |
| | 3 | Ramp | −40 | 60 | 10 |
| | 4 | Hold | −40 | 60 | 2940 |
| Secondary | 5 | Ramp | 20 | 60 | 120 |
| drying | 6 | Hold | 20 | 60 | 360 |
| | 7 | Aetate with | 20 | Atmospheric | N/A |
| | | N₂ | | | |

The lyophilized formulations were rehydrated with 20 μL aliquots of extracted RNA/DNA from a sample, as in example 6. The sample aliquots were spikes with SARS-CoV-2 RNA at various concentrations ranging from 10 to 2×10⁴ copies per uL.

A pH change was recorded as from a starting pH of 8.30-8.45 to an end pH of 7.45-7.60. The rehydrated compositions had a pH of 8.30-8.45. LAMP reaction was carried out with heating to 63° C. for 25 min. A pH change was recorded, with the end pH of the reaction mixtures after LAMP reaction being 7.45-7.60. A color change from red to green could also be observed visually.

### Example 8—Compositions with Alternative Cryoprotectants

In an alternative protocol, ficoll 400 and mannitol as described in example 6 were replaced by dextran and trehalose. The compositions of the pre-lyophilisation formulations where the same as those described in example 6, aside from replacement of ficoll 400 and mannitol with dextran and trehalose at a concentration. Trehalose was provided from a 50% w/v stock concentration, with 0.8 μL added to the 10 μL pre-lyophilization formulation to give a concentration of 4% w/v in the pre-lyophilization formulation. Dextran was provided from a 200% w/v stock concentration, with 2 μL added to the 10 μL pre-lyophilization formulation to give a concentration of 4% w/v in the pre-lyophilization formulation.

Lyophilization was performed both by an annealing and a non-annealing process, in line with the protocols in tables 17 and 18 below.

TABLE 17

| | | | Shelf | | |
|---|---|---|---|---|---|
| | | | temper- | | |
| | | Descrip- | ature | Pressure | Time |
| Stage | Step | tion | (° C.) | (μBar) | (minutes) |
| Flash freeze | 1 | Loading | −50 | Atmospheric | N/A |
| Flash freeze | 2 | Hold | −50 | Atmospheric | 120 |
| Annealing | 3 | Ramp | −15 | Atmospheric | 70 |
| | 4 | Hold | −15 | Atmospheric | 120 |
| | 5 | Ramp | −50 | Atmospheric | 70 |
| | 6 | Hold | −50 | Atmospheric | 60 |
| Primary drying | 7 | Hold | −50 | 20 | 3048 |
| Secondary | 8 | Ramp | 20 | 20 | 150 |
| drying | 9 | Hold | 20 | 60 | 360 |
| | 10 | Aerate with | 20 | Atmospheric | N/A |
| | | N₂ | | | |

TABLE 18

| | | | Shelf | | |
|---|---|---|---|---|---|
| | | | temper- | | |
| | | Descrip- | ature | Pressure | Time |
| Stage | Step | tion | (° C.) | (μBar) | (minutes) |
| Flash freeze | 1 | Loading | −50 | Atmospheric | N/A |
| Flash freeze | 2 | Hold | −50 | Atmospheric | 120 |
| Primary drying | 3 | Hold | −50 | 20 | 3280 |
| Secondary | 4 | Ramp | 20 | 20 | 150 |
| drying | 5 | Hold | 20 | 60 | 360 |
| | 7 | Aerate with | 20 | Atmospheric | N/A |
| | | N₂ | | | |

As in example 6, the lyophilized compositions were rehydrated with 20 μL sample taken from a swab spiked with SARS-CoV-2 RNA. Real-time data amplification data was visualized with Syto-9 dye added on reaction rehydration.

Reactions were color out in 8-tube strips, with a tube order as follows:
Col ctrls=color control assay (first 3 tubes)
Cov-2 std=Covid assay (19.5 mM NaOH)
Cov-2 high=Covid assay (24.5 mM NaOH)
Human=Human assay (19.5. mM NaOH)
IC 200=Internal control assay with 200 copies of internal control template (19.5 mM NaOH)
IC 2000=Internal control assay with 2000 copies of internal control template (19.5 mM NaOH)

NaOH concentrations are those of the in pre-lyophilization formulations.

Nucleic acid amplification was observed for all formulations, with show in the TTP (time to positive) data in FIG. 4.

### Example 9—Ranges of Lyophilized LAMP Components

The composition set out below in table 19 is provided to set out ranges of components for an exemplary possible colorimetric lyophilised LAMP reaction. The concentrations represent the concentrations of a lyophilised composition after rehydration:

TABLE 19

| | |
|---|---|
| Reagent | Concentration |

| | | |
|---|---|---|
| Mannitol, if present | about 1-10 (e.g. 1-4) | w/v % |
| Ficoll 400, if present | about 0.5-5 | w/v % |
| | (e.g. 0.5-2) | |
| Trehlose if present | about 1-10 | w/v % |
| Dextran if present | about 0.5-10 | w/v % |
| | (e.g. 0.5-4) | |
| MgSO4 | about 4-10 | mM |
| dNTP mix | about 1-2 | mM |
| | (e.g. 1.2-1.7) | |
| BSA | about 0-5 (e.g. 0-1.2) | mg/mL |
| DNA polymerase (e.g. | about 0.3-2 | U/μL |
| Bst 2.0 WarmStart | (e.g.0.3-0.7) | |
| Reverse transcriptase | about 0.15-5 | U/μL |
| (e.g. RTx Warmstart) | (e.g. 0.15-3.25) | |
| Phenol Red, if present | about 50-100 | μM |
| Thymol Blue, if present | about 50-175 | μM |
| EBT, if present | about 50-100 | μM |

The composition also comprises NaOH, at a concentration to provide a target pH as recited herein, for example of about 7.5-9, preferably about 7.8 to 8.5 in the rehydrated composition. The composition may also have a low buffering capacity. Tris buffer (e.g. TrisHCl) may be absent or present at a concentration of no more than about 7.5, 2.5, 1, 0.75 or 0.5 mM.

In preferred compositions, mannitol and ficoll or trehalose and dextran are present.

Primers will also be present in the composition. For example, in a pre-lyophilisation formulation (at a 2× concentration compared to the concentrations above) primer solution may be provided at a volume of up to 10% v/v (i.e. up to 1 μL primer solution in a 10 μL volume pre-lyophilisation formulation. The primer solution volume may be 3-7% in the pre-lyophilisation formulation.

Any one or more of the following optional additional additives may also be present at the following concentrations as shown in table 20 (again, the concentrations represent the concentrations of a lyophilised composition after rehydration)

TABLE 20

| Component | Concentration |
|---|---|
| Triton X-100 | about 0-5%, 0-3%, or 0-2% |
| Tween 20 | about 0-2%, or 0.1-1.8% |
| Tergitol | about 0-5%, 0-3%, 0-2%, or 0-1.5% |
| Brij-35 | about 0-10%, 0-5%, or 0-4% |
| DMSO | about 0-4% |
| Formamide | about 0-5%, 0-4%, or 0-2% |
| Sodium Azide | about 0-0.04% |
| Spermidine | about 0-0.1% |
| Polyethyleneglycol | about 0-7% |
| 35K | |
| Polyethyleneglycol | about 0-10% |
| 8K | |
| GuHCl | about 0-40 mM |
| GuTC | about 0-20 mM |
| GuHCl | about 0-40 mM |
| Glycerol | about 0-2%, 0-1%, 0-0.5% |
| KCl | about 0-110 mM, 0-100 mM, 0-70 mM, |
| | 30-70 mM, or 40-60 mM |
| (NH₄)₂SO₄ | about 0-20 mM or 5-15 mM |

wherein all percentages are % w/v.

The compositions described herein may be prepared from a pre-lyophilisation formulation at a 2× concentration compared to the concentrations of the rehydrated formulation (a 1× concentration). Accordingly, i.e. an exemplary pre-lyophilisation formulation comprises components as listed above at a 2× concentration, i.e. double the concentration listed above. In the pre-lyophilisation formulation, NaOH may be provided at a concentration of about 10-40 nM, preferably 17.5-28 mM, giving an assumed 1× concentration of about 5-20 (e.g. 8.75-14) nM.

### Example 10—Composition for Under 24 Hour Freeze Drying Protocol

In an alternative protocol, the composition comprises NaOH at a concentration to provide a target pH in the pre-lyophilisation formulation as recited herein, for example of about 9.5-10.7, preferably 10-10.5. The composition set out below is provided to accommodate large pH drops from pre-lyophilisation formulation to the final lyophilized formulations and/or rehydrated compositions when using alternative freeze drying protocols, for example an under 24 h freeze drying cycle. In the pre-lyophilisation formulation, NaOH may be provided at a concentration of about 12.5-30 nM, preferably 14-20 mM.

The target pH of the pre-lyophilisation formulation may change when using an alternative composition/formulation (both when altering the final concentration of one of the reagents or using alternative reagents), for example when using Mannitol and Ficoll instead of Trehalose and Dextran (alternative lyoprotectants); or when using Eriochrome Black T and Thymol Blue instead of Phenol Red (alternative colorimetric dyes); or when having a different final concentration (1×) of Phenol Red.

The target pH in the pre-lyophilisation formulation may be adjusted such that the post-lyophilisation resuspension pH is above the transition pH range of the dye and formulation being used.

This pre-lyophilisation formulation is summarised in Table 21:

TABLE 21

| | | | | | Volume |
|---|---|---|---|---|---|
| | | | Final conc. in pre- | | per rxn |
| | | | lyophilisation | | (μL), |
| Reagent | Stock conc. | | formulation (1X) | | 20 μL |
| Nuclease-free | | | | | 8.15 |
| Water | | | | | |
| Custom buffer | 10 | x | 1 | x | 2 |
| Trehalose | 50 | % | 4 | % | 1.60 |
| Dextran | 20 | % | 4 | % | 4.00 |
| MgSO4 | 100 | mM | 5 | mM | 1.00 |
| NaOH | 500 | mM | 15 | mM | 0.60 |
| dNTP mix | 25 | mM | 1.4 | mM | 1.12 |
| BSA glycerol-free | 40 | mg/mL | 1.2 | mg/mL | 0.60 |
| Phenol Red | 2500 | μM | 50 | μM | 0.40 |
| Bst 2.0 WarmStart | 120 | U/μL | 0.5 | U/μL | 0.08 |
| RTx Warmstart | 75 | U/μL | 0.45 | U/μL | 0.12 |
| CoV2-PM | | | | | 0.33 |

The pre-lyophilisation formulations were then dispensed in 20 μL aliquots into to 8-tube strips of 200 μL capacity surrounded by ice packs and subjected to lyophilisation. Here, the lyophilisation protocol has been optimized to be under 24 h and was as shown in the freeze drying recipe outlined in table 22 below:

TABLE 22

| | | | Shelf | | |
|---|---|---|---|---|---|
| | | | temper- | | |
| | | Descrip- | ature | Pressure | Time |
| Steps | Step | tion | (° C.) | (μBar) | (minutes) |
| Thermal | Loading | Hold | −40 | Atmospheric | N/A |
| treatment | 1 | Hold | −40 | Atmospheric | 120 |
| Drying | 2 | Hold | −40 | 180 | 15 |
| | 3 | Ramp | −30 | 180 | 20 |
| | 4 | Hold | −30 | 180 | 700 |
| | 5 | Ramp | +20 | 180 | 110 |
| | 6 | Hold | +20 | 180 | 360 |

The lyophilized formulations were rehydrated with 20 μL aliquots of extracted RNA/DNA from a sample. The sample aliquots were spiked with SARS-CoV-2 RNA at various concentrations ranging from 0 to 2×10⁴ copies per L in eight tubes, as shown in Table 23:

TABLE 23

| | SARS-CoV-2 RNA amount |
|---|---|
| Tube | (copies/reaction) |
| 1 | 0 (negative control) |
| 2 | 312.5 |
| 3 | 625 |
| 4 | 1,250 |
| 5 | 2,500 |
| 6 | 5,000 |
| 7 | 10,000 |
| 8 | 20,000 |

A pH change was recorded as from a starting pH of 8.10-8.27 (rehydrated compositions) to an end pH of 6.35-6.60 (reaction mixtures after LAMP reaction). A color change from pink (negative) to yellow (positive) was observed visually for all of tubes 2-8 (positive reactions) after 30 min incubation at 63° C.

Table 24 shows pH measurements of the formulation described in Table 21 at different stages from pre-lyophilisation formulation to after LAMP reaction using three different final concentrations of NaOH in the pre-lyophilisation formulation:

TABLE 24

| | | | pH pre- | | |
|---|---|---|---|---|---|
| NaOH final | | | lyophilisation | | |
| conc. in | | | formulation | | |
| pre- | | pH pre- | after one | | pH |
| lyophi- | | lyophi- | freeze-thaw | pH of | after |
| lisation | | lisation | cycle, required | rehydrated | LAMP |
| formulation | | formulation | for shipping | compositions | reaction |
| 15 | mM | 10.32 | 9.66 | 7.915-8.035 | 6.390 |
| 17.5 | mM | 10.46 | 9.99 | 8.375-8.435 | 6.475 |
| 20 | mM | 10.71 | 10.01 | 8.740-8.810 | 7.800 |

### Example 11—Packaging and Storage Conditions

Benefits of the provision of a lyophilized composition for use in a colorimetric nucleic acid amplification reaction include enabling stable transportation and storage at ambient temperatures. Specific storage conditions have been developed to optimise long-term stability of the lyophilized compositions at ambient temperatures. Firstly, the lyophilized composition, immediately after lyophilization, should be transferred as quickly as possible into an inert gas environment for packaging. For lyophilized compositions lyophilized in situ in a vessel, the vessel containing the lyophilized composition should be transferred into said inert gas environment. For example, the inert gas may be argon (Ar). Other examples of inert gases are helium (He), neon (Ne), krypton (Kr), xenon (Xe), and radon (Rn). In many lyophilization processes it is also common to use nitrogen gas (N₂), which is significantly inert/non-reactive at normal temperatures and pressures. It has been determined that the use of an inert gas such as argon (Ar), helium (He), neon (Ne), krypton (Kr), xenon (Xe), and radon (Rn) is beneficial to long term stability compared to the use of nitrogen gas.

The inert gas environment importantly maintains not only a low humidity, but also a low percentage of non-inert gasses, such as oxygen (O₂). These parameters should be monitored constantly throughout the packaging process to ensure they are below acceptable levels. Once in this environment, the vessels containing the lyophilized composition can be closed as quickly as possible, and optionally placed inside a secondary packaging. This secondary packaging can, for example, be a heat-sealable foil pouch. Before heat-sealing the pouch, desiccant should be placed in the pouch (or other secondary packaging), in order to maintain a low humidity environment throughout the storage duration. This entire process, including sealing or heat-sealing the secondary packaging, should be carried out in the inert gas environment, in order to ensure that inert gas has completely filled both the lyophilized composition containing vessels and the volume within the secondary packaging.

This storage process ensures the long term stability of the colorimetric performance of the composition. Without use of an inert gas, such as argon (Ar), components of the lyophilized composition, such as NaOH, even in a substantially dry state, could slowly react of with non-inert gases that remain in either the sealed vessels, or in the sealed secondary packaging. This reaction activity could result in a lower than expected/ideal starting pH of the resuspended lyophilized composition during use. In the example of using phenol red dye for the colorimetric amplification indicator, this could result in a starting color too similar to the intended end-point color of yellow, instead of the intended start-point color of pink. This effect has been observed through use of nitrogen gas (N₂) in the packaging environment. It should also be noted that while the colorimetric performance was affected as described above through use of nitrogen gas (N₂) in the packaging environment, the LAMP reaction performance was not affected.

While various specific embodiments have been illustrated and described, the above specification is not restrictive. It will be appreciated that various changes can be made without departing from the spirit and scope of the disclosure. Many variations will become apparent to those skilled in the art upon review of this specification.

1. A lyophilized composition comprising
nucleic acid amplification reagents;
a cryoprotectant;
a pH adjusting agent; and
a colorimetric amplification indicator.

2. The composition of claim 1, wherein the nucleic acid amplification reagents comprise: a nucleic acid polymerase; a nucleotide triphosphate mixture; at least two primers directed to a target nucleic acid; a source of a metal cofactor for the nucleic acid polymerase, and optionally a reverse transcriptase.

3. The composition of claim 2, wherein the source of a metal cofactor for the nucleic acid polymerase is a metal salt, for example a divalent metal salt, preferably a Mg²⁺, Ca²⁺ or Mn²⁺ salt, more preferably a magnesium salt.

4. The composition of claim 1, wherein the colorimetric amplification indicator is a pH sensitive indicator or a metal sensitive indicator, or a combination of one or more pH sensitive and metal sensitive indicators.

5. The composition of claim 1, wherein the colorimetric amplification indicator is:
(a) a pH indicator selected from gentian violet, malachite green, thymol blue, methyl yellow, bromophenol blue, congo red, methyl orange, methyl orange bromocresol green, methyl red, methyl Purple, azolitmin red, bromocresol purple, bromothymol blue, phenol red, neutral red, naphtholphthalein, Cresol red, Cresolphthalein, Phenolphthalein, Thymolphthalein, Alizarine Yellow R yellow or Indigo carmine;
(b) a metal sensitive indicator selected from hydroxynaphthol blue, eriochrome black t, calmagite, curcumin, fast sulphon black, hematoxylin, murexide, xylenon orange, BAPTA, BAPTA AM, BTC, BTC AM, Calcein, Calcein AM, Calcein Blue, Calcium Green 1, Calcium Green 2, Calcium Green 5N, Coelenterazine, Coelenterazine cp, Coelenterazine f, Coelenterazine h, Coelenterazine hep, Coelenterazine n, CoroNa Green, Corona Green AM, CoroNa Red, DAF FM, Fluo 3, Fluo 3 AM, PBFI AM, Phen Green SK, Quin 2, Quin 2 AM, RhodZin 3, hydroxynaphthol blue or calmagite; or
(c) a combination of any two or more of these indicators.

6. The composition of claim 1, wherein the colorimetric amplification indicator is phenol red, thymol blue, Eriochrome Black T, or a combination thereof, optionally wherein i) the composition comprises both a pH sensitive indicator and a metal sensitive indicator, for example thymol blue and Eriochrome Black T; or ii) wherein the composition comprises phenol red.

7. The composition of claim 1, wherein the pH adjusting agent comprises a base or any other compound that provides an overall pH raising effect when dissolved in water.

8. The composition of claim 1, wherein the pH adjusting agent is a metal hydroxide (e.g. NaOH or KOH), ammonia, calcium carbonate, sodium acetate, potassium cyanide or sodium sulfide, or a mixture thereof.

9. The composition of claim 1, wherein the composition comprises at least one cryoprotectant selected from a sugar reagent and a polyhydroxy reagent.

10. The composition of claim 1, wherein the composition comprises at least one cryoprotectant that is a surfactant, for example a sugar-based or sugar alcohol-based surfactant.

11. The composition of claim 1, wherein the composition comprises at least one cryoprotectant selected from trehalose, dextran, mannitol, a polymer formed by copolymerisation of sucrose and epichlorohydrin (such as. ficoll, e.g ficoll 400), sucrose and sorbitol, for example wherein composition comprises a combination of mannitol and ficoll or a combination of dextran and trehalose.

12. The composition of claim 1, wherein:
a) the pH adjusting agent is present in an amount sufficient to produce a rehydrated reaction mixture with a target starting pH when the composition is rehydrated with water, wherein the target starting pH is about 7.5-9, preferably about 7.5-8.5, more preferably about 7.8 to 8.5; and/or
b) the composition is capable of being rehydrated to provide a rehydrated formulation comprising:
i) the source of a metal cofactor (e.g. MgSO₄) at a concentration of about 4-10 mM, 4-8 mM, 6-8 mM, 4-7 mM or 6-7 mM; and
ii) the pH adjusting agent (e.g. NaOH) at a concentration sufficient to provide a pH of about 7.5-9, 7.5-8.5, or 7.8 to 8.5; or
c) i) the source of a metal cofactor (e.g. MgSO₄) at a concentration of about 4-10 mM, 4-8 mM, 6-8 mM, 4-7 mM or 6-7 mM; and
ii) the pH adjusting agent (e.g. NaOH) at a concentration of at least about 5 mM, at least about 6 mM, at least about 7 mM, at least about 8 mM, at least about 8.5 mM, or at least about 8.75 mM.

13. The composition of claim 12, wherein the composition is capable of being rehydrated to provide a rehydrated formulation comprising a cryoprotectant concentration of at least about 0.5% w/v, at least about 1% w/v or at least about 1.5% w/v, up to 20% w/v or up to 15% w/v, optionally wherein the composition is capable of being rehydrated to provide a rehydrated formulation comprising at least one of:
a) a mannitol concentration of about 1-10% w/v, 1-5% w/v or 1-4% w/v,
b) a Ficoll concentration of about 0.5-5% w/v, 0.5-3% w/v or 0.5-2% w/v;
c) a trehalose concentration of about 1-10% w/v, 1-5% w/v or 1-4% w/v; and
d) a dextran concentration of about 0.5-10% w/v, 0.5-5% w/v or 0.5-4% w/v.

14. The composition of claim 12, wherein the composition is capable of being rehydrated to provide a rehydrated formulation comprising any one or more of:
a) the colorimetric amplification indicator at a concentration of at least about 50 μM, for example about 50-300 μM;
b) phenol red at a concentration of about 50-100 μM or thymol blue at a concentration of about 50-175 μM and EBT at a concentration of about 50-100 μM;
c) a nucleic acid polymerase at a concentration of at least about 0.3 U/μL, 0.3-2 U/μL, 0.3-1 U/μL or 0.3-0.7 U/μL; and/or
d) a reverse transcriptase enzyme, if present, at a concentration of at least about 0.15 U/μL, a concentration of about 0.15-5 U/μL, or a concentration of about 0.15-3.25 U/μL; and/or
e) a nucleotide triphosphate mixture at a concentration of at least 1 mM or about 1 to 2 mM or 1.2 to 1.7 mM; and/or
f) a glycerol concentration of about 0-2% w/v, 0-1% w/v or 0-0.5% w/v;
and/or
g) a buffering capacity of no more than that provided by a 7.5 mM solution of Tris buffer with a starting pH of 8.8, no more than that provided by a 5 mM solution of Tris buffer with a starting pH of 8.8, no more than that provided by a 2.5 mM solution of Tris buffer with a starting pH of 8.8, no more than that provided by a 1 mM solution of Tris buffer with a starting pH of 8.8 or no more than that provided by a 0.5 mM solution of Tris buffer with a starting pH of 8.8.

15. The composition of claim 1, wherein:
a) the composition further comprises an enzyme stabiliser, preferably selected from bovine serum albumin (BSA), DMSO, gelatin, Prionex (a polypeptide fraction of highly purified dermal collagen of porcine origin), sodium alginate, or a combination thereof, and/or
b) the composition further comprises at least one additive selected from Triton X-100, KCl, (NH₄)₂SO₄, Tween 20, sodium azide, spermidine, tergitol, Brij-35, DMSO, formamide, PEG 35K, PEG 8K, GuHCl, GuTC, dithiothreitol (DTT) and ethylenediaminetetraacetic acid (EDTA).

16. The composition of claim 1, wherein the composition is obtainable by lyophilisation of a pre-lyophilisation formulation comprising the nucleic acid amplification reagents; surfactant; pH adjusting agent; and colorimetric amplification indicator, and water (e.g. nuclease-free water).

17. The composition of claim 16, wherein the pre-lyophilisation formulation comprises:
a) the pH adjusting agent (e.g. NaOH) and a source of a metal cofactor (e.g. MgSO₄) present at a molar ratio of at least 1:1 (pH adjusting agent:cofactor), preferably wherein the pH adjusting agent is present in a molar excess compared to the source of metal cofactor, preferably wherein the pH adjusting agent:cofactor molar ratio is at least 1.05:1; and/or
b) a source of a metal cofactor (e.g. MgSO₄) at a concentration of about 8-20 mM, 4-16 mM, 12-20 mM, 8-14 mM or 12-14 mM; and/or
c) the pH adjusting agent (e.g. NaOH) at a concentration of at least about 10 mM, at least about 12 mM, at least about 14 mM, at least about 16 mM, at least about 17 mM, at least about 18 mM, or at least about 20 mM, for example wherein the pH adjusting agent is at a concentration of no more than 40 mM, no more than 30 mM, preferably no more than 27 mM; and/or
d) a cryoprotectant concentration of at least about 1% w/v, at least about 2% w/v or at least about 3% w/v, for example wherein the cryoprotectant concentration is up to about 40% w/v or up to about 30% w/v; and/or
e) at least one of: a) a mannitol concentration of about 2-20% w/v, 2-10% w/v, 2-8% w/v, 2-6% w/v or 4% w/v; b) a Ficoll concentration of about 1-10% w/v, 1-6% w/v, 1-4% w/v, 1-3% w/v or 2% w/v; c) a trehalose concentration of about 2-20% w/v, 2-10% w/v, 2-8% w/v, 2-6% w/v or 4% w/v; and d) a dextran concentration of about 1-20% w/v, 1-10% w/v, 1-8% w/v, 2-6% w/v or 4% w/v; and/or
f) mannitol and ficoll, or dextran and trehalose; and/or
g) the colorimetric amplification indicator at a concentration of at least about 100 μM, for example about 100-600 μM; and/or
h) i) phenol red at a concentration of about 100-200 μM; or ii) thymol blue at a concentration of about 100-350 μM and EBT at a concentration of about 100-200 μM; and/or
i) the nucleic acid polymerase at a concentration of at least about 0.6 U/μL, 0.6-4 U/μL, 0.6-2 U/μL, 0.6-1.4 U/μL or 1 U/μL; and/or
a. the reverse transcriptase enzyme, if present, at a concentration of at least about 0.3 U/μL, a concentration of about 0.3-10 U/μL, a concentration of about 0.6-6.5 U/μL, or a concentration of about 0.9 U/μL; and/or
b. the nucleotide triphosphate mixture at a concentration of at least about 2 mM, or about 2 to 4 mM, 2.4 to 3.4 mM or 2.8 mM; and/or
j) an enzyme stabiliser, for example wherein the enzyme stabiliser is bovine serum albumin (BSA), DMSO, gelatin, Prionex (a polypeptide fraction of highly purified dermal collagen of porcine origin), sodium alginate, or a combination thereof, preferably BSA, optionally at a concentration of about 0-20 mg/mL, 0-10 mg/mL, 1-20 mg/mL, 1-10 mg/mL, 2-20 mg/mL, 2-10 mg/mL, 2-6 mg/mL, 2-4 mg/mL or 2.4 mg/mL; and/or
k) at least one additive, selected from Triton X-100, KCl, (NH₄)₂SO₄, Tween (polyoxyethylene (20) sorbitan monolaurate), sodium azide, spermidine, tergitol, Brij-35, DMSO, formamide, PEG 35K, PEG 8K, GuHCl, GuTC, dithiothreitol (DTT) and ethylenediaminetetraacetic acid (EDTA); and/or
l) a glycerol concentration of no more than about 4% w/v, no more than about 2% w/v or no more than about 1% w/v; and/or
m) a Tris buffer (e.g. TrisHCl) concentration of 0-15 mM, 0-5 mM, 0-2 mM, 0-1.5 mM or 0-1 mM.

18. The composition of claim 17, wherein the pre-lyophilisation formulation is provided:
a) in a diluted form, wherein the pre-lyophilisation formulation comprises the same components as claim 17 at the same relative concentrations, but with the concentrations of all components reduced by the same factor; or
b) in a concentrated form, wherein the pre-lyophilisation formulation comprises the same components as claim 17, at the same relative concentrations, but with the concentrations of all components increased by the same factor.

19. The composition of claim 16, wherein the pre-lyophilisation formulation has a pH of at least about 8.5, for example about 8.5-12, about 8.5-11, or about 8.5-10.5, preferably at least about 9 or at least about 9.5, for example about 9.5-10.5.

20. A lyophilised composition for use as a colour control composition in a colorimetric nucleic acid amplification reaction, wherein the composition corresponds to a composition of claim 1, but with the exclusion of at least one nucleic acid amplification reagent.

21. A process for producing a lyophilized composition for use in a nucleic acid amplification reaction, the process comprising:
a. providing a pre-lyophilisation formulation comprising nucleic acid amplification reagents, a cryoprotectant, a base and a colorimetric amplification indicator in water; and
b. lyophilizing the formulation to produce a lyophilized composition. optionally wherein the pre-lyophilisation formulation is as defined in claim 17.

22. A process for producing a lyophilized composition for use as a colour control composition in a colorimetric nucleic acid amplification reaction, the process corresponding to the process of claim 21, but wherein no nucleic acid polymerase and/or reverse transcriptase is present in the pre-lyophilisation formulation.

23. A lyophilized composition produced by a process claim 21.

24. A method for determining the presence of a target nucleic acid in a test sample, the method comprising:
a. providing a lyophilised composition according to claim 1 in a first vessel;
b. rehydrating the lyophilised composition within the first vessel and adding the test sample to the first vessel to provide a rehydrated reaction mixture;
c. subjecting the rehydrated reaction mixture to conditions suitable to allow nucleic acid amplification to occur if the target nucleic acid is present in the sample;
d. determining a positive result for the presence of a target nucleic acid in the sample if a colorimetric change occurs due to the presence of nucleic acid amplification.

25. The method of claim 24, wherein:
a) the test sample comprises extracted nucleic acids (RNA and/or DNA) from a biological or environmental sample in an eluent, optionally wherein the biological sample is a nasopharyngeal swab, or a blood, a urine, a saliva, a tissue, stool, serum, lymph, semen, sweat, tears, amniotic fluid or wound exudate sample, or any other bodily fluid and the environmental sample is water, such as waste water; and/or
b) the target nucleic acid is a nucleic acid sequence from a pathogen, for example SARS-CoV-2, influenza, respiratory syncytial virus (RSV), human rhinovirus (HR), adenovirus or parainfluenza.

26. The method of claim 24, wherein rehydration of the lyophilised reaction mixture and addition of the test sample are carried out as a single step, by addition of extracted RNA or DNA within an eluent to lyophilised reaction mixture.

27. A kit for carrying out a nucleic amplification reaction, the kit comprising a vessel having a lyophilised composition claim 1 contained therein.
